SEQUENCE LISTING (1 sequence)

SEQ ID NO: 1 - DNA, 11 bp, synthetic construct
  source 1..11: mol_type = other DNA
aatccgttgga

(19) United States
(12) Patent Application Publication
(10) Pub. No.: US 2026/0226523 A1
(43) Pub. Date: Aug. 6, 2026

(54) INKJET-ASSISTED ENZYMATIC NUCLEIC ACID SYNTHESIS

(71) Applicant: DNA SCRIPT, LE KREMLIN-BICETRE (FR)

(72) Inventors: Nicolas MOGHADDAM, Le Kremlin-Bicêtre (FR); Beatrice ADELIZZI, Le Kremlin-Bicêtre (FR); Tessa LOMAN, Le Kremlin-Bicêtre (FR); Damiano VERARDO, Le Kremlin-Bicêtre (FR); Daniel RODRIGUEZ-PINZON, Le Kremlin-Bicêtre (FR); Adrian HORGAN, Le Kremlin-Bicêtre (FR)

(21) Appl. No.: 19/148,524

(22) PCT Filed: Jan. 16, 2024
(86) PCT No.: PCT/EP2024/050932

§ 371 Date: Jul. 16, 2025

(30) Foreign Application Priority Data
Jan. 16, 2023 (EP) 23315006.9
Feb. 21, 2023 (EP) 23315037.4

(51) Int. Cl. C12P 19/34; C09D 11/38; C09D 11/54; C12N 9/12; C12N 9/14

(52) U.S. Cl. CPC C12P 19/34; C09D 11/38; C09D 11/54; C12N 9/1241; C12N 9/14; C12Y 306/01001

(57) ABSTRACT

Methods, devices, and compositions are provided for inkjet-assisted synthesis of a plurality of polynucleotides at reaction sites on a substrate using template-free polymerases, such as, terminal deoxynucleotidyl transferases (TdTs). Compositions include printable stabile formulations of synthesis reagents for inkjet delivery including, but not limited to, template-free polymerases, divalent cations, and nucleotides.

### INTRODUCTION

Inkjet printing is a low-cost versatile technology for non-contact delivery of defined quantities of liquids to precise locations with minimal wastage. The technology has been applied to synthesis of oligonucleotide microarrays using phosphoramidite chemistry and has been employed to directly print enzymes onto substrates in the production of enzyme-based biosensors. In regard to the latter applications of inkjet printing, it has been observed that not only is enzyme activity affected by shear forces and the rheological requirements for droplet formation, but also by the changing enzyme concentration and buffer conditions from evaporative loss when, for example, enzyme-containing fluids are printed to microarrays, e.g. Derby (cited above, 2008); Di Risio et al, Macromolecular Rapid Comm., 28(18-19): (2007); Nishioka et al, J. Amer. Chem. Soc., 126(50): 16320-16321 (2004).

Recently there has been an interest in applying enzyme-based polynucleotide synthesis to problems which are ill-suited for conventional chemically based DNA synthesis, largely because of the mild aqueous reaction conditions of the enzymatic process. However, in addition to the above-mentioned difficulties of inkjet-delivery of enzymes, the use of enzymes presents a host of additional problems for any automated multi-step synthesis process including, enzyme adhesion to surfaces, the need for stringent temperature and pH control to maintain enzyme activity, aggregation of enzymes resulting in loss of activity and/or clogging of tubing, reaction sites or nozzles, variations in enzyme activity in or near synthesis supports, batch to batch differences in enzyme specific activity, the formation of foams or bubbles that inhibit reagent transfer and separation, loss of efficiency from reaction of certain protection groups with environmental contaminants, such as formaldehyde, and the like.

If the above challenges could be overcome, the ability to carry out inkjet reagent delivery for enzyme-based synthesis of dense arrays of polynucleotides would provide not only a convenient desk top synthesis method using aqueous reagents without the need for extensive environmental controls, but also significant advances in several diverse fields, including DNA data storage and cell and tissue analysis, such as, by direct labeling of viable biological cells, direct synthesis of spatial barcodes on tissues, and the like.

### SUMMARY OF THE INVENTION

Methods, devices, and compositions are provided for inkjet-assisted synthesis of a plurality of polynucleotides at reaction sites on a substrate using template-free polymerases, such as, terminal deoxynucleotidyl transferases (TdTs). Compositions include printable stabile formulations of synthesis reagents for inkjet delivery including, but not limited to, template-free polymerases, divalent cations, and nucleotides.

In one aspect, a method of enzymatically synthesizing a plurality of polynucleotides each having a predetermined sequence at reaction sites on a substrate is provided, the method comprising: (a) providing the substrate, wherein the substrate comprises initiators at a plurality of reaction sites, wherein each initiator has a free 3′-hydroxyl group, and wherein each polynucleotide of the plurality is assigned to a reaction site for synthesis; (b) providing a printable reagent composition comprising: a template-free polymerase, a divalent cation, a 3′-O-protected nucleoside triphosphate, and a polar organic solvent that reduces DNA melting temperature; (c) performing a reaction cycle comprising the steps of i) dispensing through one or more inkjet printhead nozzles at least one droplet of the printable reagent composition to each reaction site of the plurality, wherein the initiator or elongated fragments having free 3′-O-hydroxyls are reacted with the 3′-O-protected nucleoside triphosphate under suitable conditions for elongation by the template-free polymerase, wherein the initiator or elongated fragments are elongated by incorporation of the 3′-O-protected nucleoside triphosphate to form 3′-O-protected elongated fragments, and (ii) dispensing through one or more inkjet printhead nozzles at least one droplet of a deprotection solution to deprotect the 3′-O-protected elongated fragments to form elongated fragments having free 3′-hydroxyls; and (d) repeating step (c) until the plurality of polynucleotides is synthesized,
wherein the divalent cation is a cobalt divalent cation (Co2+) and the divalent cation concentration is ranging from 0.1 mM to 1 mM, preferably from 0.2 to 0.5 mM, even more preferably is about 0.25 mM and the pH of the printable reagent composition is ranging from pH 5.1 to 6.6, preferably from pH 5.6 to 6.6, preferably from pH 5.6 to 6.5, more preferably from pH 5.6 to 6.4, even more preferably from pH 6.0 to 6.4, even more preferably the pH is about 6.0, or
wherein the divalent cation is a manganese divalent cation (Mn2+) and the divalent cation concentration is ranging from 0.5 mM to 7 mM, preferably from 1 to 6 mM, even more preferably is about 1.25 mM and the pH of the printable reagent composition is ranging from pH 6.0 to 7.0, preferably from pH 6.4 to 6.8, more preferably the pH is about 6.6, or
wherein the divalent cation is magnesium divalent cation (Mg2+) and the divalent cation concentration is ranging from 1 mM to 20 mM, preferably from 2 to 15 mM, preferably from 3 to 8 mM, even more preferably is about 5 mM and the pH of the printable reagent composition is ranging from pH 5.6 to 8.4, preferably from pH 7.0 to 8.2, more preferably the pH is about 8.0.

In some embodiments, the printable reagent composition is stored at a temperature in a range from −20° C. to 4° C. prior to said dispensing. In some embodiments, the printable reagent composition is stored at a temperature in a range from −20° C. to 4° C. for up to a month prior to said dispensing.

In some embodiments, the 3′-O-protected nucleoside triphosphate has a concentration of 100 μM to 2000 μM.

In some embodiments, the template-free polymerase has a concentration of 5 μM to 30 μM.

In some embodiments, the 3′-O-protected nucleoside triphosphate comprises a 3′-aminooxy protecting group.

In some preferred embodiments, the buffer is cacodylic acid, HEPES, Tris or MES.

In some embodiments, the printable reagent composition comprises 20 μM terminal deoxynucleotidyl transferase, 500 μM 3′-O-protected nucleoside triphosphate, 0.25 mM CoCl₂, 15% DMSO, 50 mM O-benzylhydroxylamine hydrochloride, 10% glycerol, and 0.05% polyoxyethylene (20) sorbitan monolaurate.

In some embodiments, the dispensing of the printable reagent composition is performed at room temperature.

In some embodiments, the droplet has a volume ranging from 1 pL to 30 pL.

In some embodiments, the substrate ranges in size from 10 cm² to 1 m².

In some embodiments, the substrate is on a planar surface.

In some embodiments, the substrate is on a non-planar surface of a bead or particle.

In some embodiments, the printable reagent composition is filtered through a filter before said dispensing. In some embodiments, the filter has a pore size less than or equal to 0.8 m in diameter. In some embodiments, the filter comprises polytetrafluoroethylene.

In some embodiments, the printable reagent composition is prepared by a method comprising: mixing the divalent cation, the polar organic solvent, and the buffer to form a solution; filtering the solution through a first filter to form a filtered solution; adding the template-free polymerase and the 3′-O-protected nucleoside triphosphate to the filtered solution to form the printable reagent composition; and filtering the printable reagent composition through a second filter before said dispensing. In some embodiments, the first filter has a pore size of 0.8 μm in diameter, and the second filter has a pore size of 5 μm in diameter.

In some embodiments, the 3′-O-protected nucleoside triphosphate has been double or triple purified prior to its addition to prepare the printable reagent composition.

In some embodiments, the printable reagent composition further comprises a non-ionic surfactant, a viscosity modifying agent, a surface tension modifying agent, an aldehyde scavenger, or a combination thereof. Preferably, any buffer, non-ionic surfactant, viscosity modifying agent, surface tension modifying agent, or aldehyde scavenger present in the printable reagent composition does not inhibit the template-free polymerase or have a free hydroxyl group that can serve as a substrate for the template-free polymerase.

In some embodiments, the non-ionic surfactant is Tween® 20 (polyoxyethylene (20) sorbitan monolaurate). In some embodiments, the Tween® 20 (polyoxyethylene (20) sorbitan monolaurate) has a concentration of less than 0.5 weight percent.

In some embodiments, the viscosity-modifying agent is also a humectant. In some embodiments, the viscosity-modifying agent is selected from the group consisting of hydroxyethyl cellulose, carboxymethyl cellulose, glycerol, and glycerol acetate. In some embodiments, viscosity-modifying agent is glycerol. In some embodiments, the glycerol or glycerol acetate has a concentration of less than 50 weight percent.

In some embodiments, the viscosity of the printable reagent compositions is less than 8 centipoises (cP) when measured at 20° C. In some embodiments, the viscosity of the printable reagent compositions ranges from 1 cP to 3 cP when measured at 20° C.

In some embodiments, the aldehyde scavenger is an O-substituted hydroxylamine.

For example, the aldehyde scavenger may include, without limitation, O-benzylhydroxylamine or O-benzylhydroxylamine hydrochloride.

In some embodiments, the printable reagent composition comprises an inorganic pyrophosphatase.

In some embodiments, the 3′-O-protected nucleoside triphosphate in the printable reagent composition has been purified, preferably double purified, even more preferentially triple purified, to remove contaminating pyrophosphate.

In some embodiments, the printable reagent composition has no exogenous source of pyrophosphate.

In some embodiments, the printable reagent composition further comprises a dye.

In some embodiments, the printable reagent composition comprises a single kind of 3′-O-protected nucleoside triphosphate and further comprises a dye that has distinct spectral characteristics by which the kind of nucleoside triphosphate can be identified.

In some embodiments, the kind of 3′-O-protected nucleoside triphosphate dispensed to a reaction site depends on the predetermined sequence of the polynucleotide assigned to the reaction site.

In some embodiments, the dispensing of the printable reagent composition is performed at a temperature above 18° C. In some embodiments, the dispensing of the printable reagent composition is performed at a temperature ranging from 18° C. to 45° C. In some embodiments, the dispensing of the printable reagent composition is performed at room temperature.

In some embodiments, the printable reagent composition is stored at a temperature in a range from −20° C. to 4° C. prior to said dispensing. In some embodiments, the printable reagent composition is stored at a temperature in a range from −20° C. to 4° C. for up to a month prior to said dispensing.

In some embodiments, the printable reagent composition has a surface tension ranging from about 15 dynes/cm to about 50 dynes/cm when measured at room temperature.

In some embodiments, the template-free polymerase is selected from the group consisting of a terminal deoxynucleotidyl transferase, a translesion DNA polymerase of type η (eta), a translesion DNA polymerase of type ζ (zeta) a polynucleotide phosphorylase (PNPase), a template-independent RNA polymerase, a terminal transferase, a template-independent DNA polymerase, a reverse transcriptase, and a 9° N DNA polymerase.

In some embodiments, the printable reagent composition is split into a first reagent composition comprising the template-free polymerase and the divalent cation; and a second reagent composition comprising the 3′-O-protected nucleoside triphosphate and the polar organic solvent. In other embodiments, the printable reagent composition is split into a first reagent composition comprising the template-free polymerase and the 3′-O-protected nucleoside triphosphate; and a second reagent composition comprising the divalent cation, and the polar organic solvent.

In some embodiments, the first reagent composition and the second reagent composition are dispensed separately through the one or more inkjet printhead nozzles.

In some embodiments, the first reagent composition and the second reagent composition are mixed prior to said dispensing.

In some embodiments, the mixing of the first reagent composition and the second reagent composition can lead to the printable reagent composition.

In some embodiments, the first reagent composition and the second reagent composition are stored at a temperature in a range from −20° C. to 4° C. prior to said dispensing.

In some embodiments, step (c) further comprises washing the reaction sites after the 3′-O-protected elongated fragments are deprotected. In some embodiments, step (c) further comprises drying said reaction sites after the 3′-O-protected elongated fragments are deprotected or after said washing.

In some embodiments, each of said reaction sites are distinct and non-overlapping with other said reaction sites.

In some embodiments, step (c) further comprises incubating the initiator or elongated fragments having free 3′-O-hydroxyls, the 3′-O-protected nucleoside triphosphate, and the template-free polymerase for a predetermined period of time.

In some embodiments, the reaction sites are imaged during said incubating.

In some embodiments, the inkjet printhead nozzles deliver the droplets to at least one of the reaction sites in a move-stop droplet delivery mode.

In some embodiments, the inkjet comprises a recirculating ink print head.

In some embodiments, step (c) further comprises capping said initiators or elongated fragments that fail to be elongated.

In some embodiments, each of said polynucleotides of said plurality is assigned to a different reaction site for synthesis.

In another aspect, a printable reagent composition is provided, the printable reagent composition comprising: a template-free polymerase, a divalent cation, a 3′-O-protected nucleoside triphosphate, and a polar that reduces DNA melting temperature,
wherein the divalent cation is a cobalt divalent cation (Co²⁺) and the divalent cation concentration is ranging from 0.1 mM to 1 mM, preferably from 0.2 to 0.5 mM, even more preferably is about 0.25 mM and the pH of the printable reagent composition is ranging from pH 5.1 to 6.6, preferably from pH 5.6 to 6.6, preferably from pH 5.6 to 6.5, more preferably from pH 5.6 to 6.4, even more preferably from pH 6.0 to 6.4, even more preferably the pH is about 6.0, or
wherein the divalent cation is a manganese divalent cation (Mn²⁺) and the divalent cation concentration is ranging from 0.5 mM to 7 mM, preferably from 1 to 6 mM, even more preferably is about 1.25 mM and the pH of the printable reagent composition is ranging from pH 6.0 to 7.0, preferably from pH 6.4 to 6.8, more preferably the pH is about 6.6, or
wherein the divalent cation is magnesium divalent cation (Mg²⁺) and the divalent cation concentration is ranging from 1 mM to 20 mM, preferably from 2 to 15 mM, preferably from 3 to 8 mM, even more preferably is about 5 mM and the pH of the printable reagent composition is ranging from pH 5.6 to 8.4, preferably from pH 7.0 to 8.2, more preferably the pH is about 8.0.

In some embodiments, the printable reagent composition is filtered through a filter having a pore size less than or equal to 0.8 μm in diameter.

In some embodiments, the buffer is cacodylic acid, HEPES, Tris or MES. In some embodiments, the printable reagent composition comprises 0.5 M cacodylic acid.

In some embodiments, the polar organic solvent has a concentration ranging from 5 volume/volume percent to 30 volume/volume percent.

In some embodiments, the printable reagent composition further comprises water, a non-ionic surfactant, a viscosity modifying agent, a surface tension modifying agent, an aldehyde scavenger, or any combination thereof.

In some embodiments, the non-ionic surfactant is polyoxyethylene (20) sorbitan monolaurate. In some embodiments, the polyoxyethylene (20) sorbitan monolaurate has a concentration of less than 0.5 weight percent.

In some embodiments, the viscosity-modifying agent is also a humectant.

In some embodiments, the viscosity-modifying agent is selected from the group consisting of hydroxyethyl cellulose, carboxymethyl cellulose, glycerol, and glycerol acetate. In some embodiments, the glycerol or glycerol acetate has a concentration of less than 50 weight percent.

In some embodiments, the viscosity of the printable reagent compositions is less than 8 centipoises (cP) when measured at 20° C. In some embodiments, the viscosity of the printable reagent compositions ranges from 1 cP to 3 cP when measured at 20° C.

In some embodiments, the aldehyde scavenger is an O-substituted hydroxylamine.

In some embodiments, the O-substituted hydroxylamine is O-benzylhydroxylamine or O-benzylhydroxylamine hydrochloride.

In some embodiments, the printable reagent composition comprises an inorganic pyrophosphatase.

In some embodiments, the 3′-O-protected nucleoside triphosphate in the printable reagent composition has been purified, preferably double purified, even more preferentially triple purified, to remove contaminating pyrophosphate.

In some embodiments, the printable reagent composition further comprises a dye.

In some embodiments, the 3′-O-protected nucleoside triphosphate comprises a 3′-aminooxy protecting group.

In some embodiments, the printable reagent composition comprises a single kind of nucleoside triphosphate and further comprises a dye that has distinct spectral characteristics by which the kind of nucleoside triphosphate can be identified.

In some embodiments, the 3′-O-protected nucleoside triphosphate is a 3′-O-protected-deoxyadenosine triphosphate, a 3′-O-protected-deoxythymidine triphosphate, a 3′-O-protected-deoxycytidine triphosphate, or a 3′-O-protected-deoxyguanosine triphosphate.

In some embodiments, the 3′-O-protected nucleoside triphosphate has a concentration of 100 μM to 2000 μM.

In some embodiments, the template-free polymerase has a concentration of 5 μM to 30 μM.

In some embodiments, the reagent composition has a surface tension ranging from about 15 dynes/cm to about 50 dynes/cm when measured at room temperature.

In some embodiments, the template-free polymerase is selected from the group consisting of a terminal deoxynucleotidyl transferase, a translesion DNA polymerase of type η (eta), a translesion DNA polymerase of type ((zeta), a polynucleotide phosphorylase (PNPase), a template-independent RNA polymerase, a terminal transferase, a template-independent DNA polymerase, a reverse transcriptase, and a 9° N DNA polymerase.

In some embodiments, the printable reagent composition comprises 20 μM terminal deoxynucleotidyl transferase, 500 μM 3′-O-protected nucleoside triphosphate, 0.25 mM CoCl₂, 15% DMSO, 50 mM O-benzylhydroxylamine hydrochloride, 10% glycerol, and 0.05% polyoxyethylene (20) sorbitan monolaurate.

In another aspect, a set of printable reagent compositions is provided, the set of printable reagent compositions comprising: a first reagent composition comprising a template-free polymerase and a divalent cation; and a second reagent composition comprising a 3′-O-protected nucleoside triphosphate and a polar organic solvent.

In another aspect, a set of printable reagent compositions is provided, the set of printable reagent compositions comprising: a first reagent composition comprising a template-free polymerase and a 3′-O-protected nucleoside triphosphate; and a second reagent composition comprising a divalent cation, and a polar organic solvent.

### BRIEF DESCRIPTION OF THE DRAWINGS

FIG. 1 shows combinations of components in inks that were stable and unstable. In dark gray (without black dots) are inks that were visually stable for >48 h at 22° C. In light gray (with black dots) are inks that were stable <48 h at 22° C. Inks that were active but unstable forming precipitate possessed nucleotide, cobalt, and TdT and/or DMSO. DMSO-free inks and TdT-free inks formed a precipitate in <48 hours, while dNTP-free and cobalt-free inks were visually stable for >>48 h. Inks not having nucleotide or cobalt are inactive.

FIG. 2 shows how ink instability (marked with a black dot) depends on pH and [Co], with and without DMSO. An effective way to prevent precipitation is to concurrently lower the ink pH (here measured for a DMSO-free analogue) and lower the [Co]. From this study, the combination of using 0.25 mM cobalt and an ink pH of 6.0 was selected (=‘Optink0.25’), as initial activity tests indicated that the enzyme lost activity when the ink pH was below 5.1 but increased at higher pH.

FIG. 3 shows effects of nucleotide and temperature on ink stability. ‘Optink0.25’ and ‘Standard’ ink were prepared as in Examples 1 and 2 for each nucleotide (T vs. G, C, A) and after different levels of nucleotide purification (single purified: 1×P; and double or triple purified: 2×P; Note that C was only available as triple purified). Stability was studied as in Examples 1 and 2 as a function of storage temperature (22° C. or 4° C.). Black dots denote solutions that were unstable. Storing ink at lower temperature has a more obvious effect on stability for ‘Optink0.25’ than for ‘Standard’ ink. Inks at 4° C. were stable for greater than 1 month, which is sufficient for potentially 10 synthesis runs. Although much less impactful than temperature, the nucleotide also has an impact on stability. Ink stability was found to be in the order T<G<A≈C. For ‘Standard’ ink at 22° C., T and G precipitate in <12 hours while C and A in <24 h. For ‘Optink0.25’, T and G precipitate approximately a couple of days before C and A (i.e., 5 vs. 7 days). The purification state of the nucleotide has a marginal effect on stability. For ‘Standard’ ink with DMSO, 2×P inks precipitate only a few hours (<24 h) after 1×P inks (<6 h) using 3′-ONH2-dTTP nucleotide. For Optink0.25, 2×P inks precipitate 2 to 3 days after 1×P inks.

FIGS. 4A-4B show effects of filtering on ink stability and activity. FIG. 4A. Filtered inks were filtered twice: once with a 0.8 μm filter before adding enzyme and nucleotide, then with a 5 μm PTFE low bind filter after adding enzyme and nucleotide. FIG. 4B. UV-Vis measurements were made without nucleotide to facilitate quantification of TdT and after dilution, to lower the absorption below 1.0. Stability at 22° C. was better for filtered ink than for non-filtered ink. No difference was observed for inks stored at 4° C. UV-Vis measurements demonstrated that the filtration protocol lowered [TdT] only very slightly (0.5-2.7% depending on the volume of ink filtered).

FIGS. 5A-5B show ink activity during a run for automated synthesis (FIG. 5A) and manual synthesis (FIG. 5B). Ink activity was tested by performing either manual EDS on DNA-functionalized glass slides, or automated EDS using a liquid handler (Tecan) on DNA-functionalized beads. A shorter length product band and smear is evident in the case of the 52mer synthesis of sequence e13 with ‘Standard’ ink. This smear is not present for the case of ‘Optink0.25’ or for the case of a shorter (21 cycle) manual synthesis with ‘Optink0.25’.

FIGS. 6A-6C show ink activity versus storage temperature. Manual synthesis was performed on ‘Standard’ ink (FIG. 6A) and ‘Optink0.25’ (FIGS. 6B-6C) to test for activity after aging at 4° C. and 20° C. After 48 hours, ‘Standard’ ink precipitated at both storage temperatures (FIG. 6A). However, the supernatant of ‘Standard’ ink stored at 4° C. was still active while the supernatant of ‘Standard’ ink stored at 20° C. gave a series of bands at shorter length, corresponding to poor purity. By comparison, ‘Optink0.25’, was visually stable at both storage temperatures and gave good purity for greater than 1 month if stored at 4° C. (FIG. 6C). At 20° C., stability was also much greater, up to almost 1 week (FIG. 6C).

FIGS. 7A-7B show HPLC monitoring of nucleotide concentration. Ion exchange IE-HPLC-UV studies were performed to monitor the decay of 3′ONH₂-dNTP (N=T or G) in the ink upon aging. ‘Standard’ ink, ‘Optink0.25’, and a control ink (i.e., Optink0.25 without CoCl₂ and TdT) were prepared, aged at either 20 or 4° C., and filtered through a centrifugal Amicon filter (3 kDa) to remove TdT. Nucleotide in control ink (ink lacking cobalt and TdT) was perfectly stable at both storage temperatures. ‘Standard’ inks aged at 20° C. and 4° C., however, showed a rapid decay of both dTTP (FIG. 7A) and dGTP (FIG. 7B). ‘Optink0.25’ stored at 20° C., by comparison, showed a significantly slower loss of dNTP, particularly when stored at 4° C. (>80% remaining after 4 weeks).

FIG. 8 shows possible pathways for generation of pyrophosphate and formation of precipitates. Although not wishing to be bound by theory, a precipitate may form by complexation of pyrophosphate with cobalt and other ligands (L). This hypothesis is supported by double purified nucleotides (2×P) giving slightly more stable inks than single purified nucleotides (1×P). We speculate that above pH 6.5 (≈pKa of dNTP) and at higher cobalt concentrations complexation is favored resulting in faster precipitation. We also speculate that the pyrophosphate is generated in situ. Normally, dNTPs degrade slowly in aqueous solution through consecutive steps of single dephosphorylation (pathway 1). In a complex aqueous solution, however, another pH-dependent pathway involving coordination by transition metal ions (M²⁺, such as Co²⁺) and nucleophilic attack of for instance, an alcohol, at the alpha position phosphate may be possible (pathway 2). This would generate pyrophosphate, whose formation might also inhibit chain extension as in the case of PCR reactions.

FIGS. 9A-9B show that removing or lowering pyrophosphate in solution prevents precipitation. Inorganic pyrophosphatase ([2.3 nM]) was added to ‘Standard’ and ‘Optink0.25’. pyrophosphatase catalyzes the hydrolysis of pyrophosphate to phosphate. FIG. 9A shows that addition of pyrophosphatase increased the visual stability of the inks confirming that precipitate formation is driven by pyrophosphate. Addition of pyrophosphatase to the ink is beneficial therefore if the aim is simply to reduce the risk of the printhead becoming blocked. It does not help, however, in terms of ink activity. This is likely due to the fact that, by consuming pyrophosphate, pyrophosphatase promotes pathway 2, which in turn hastens the consumption of the nucleotide (3′ONH₂-dNTP). This hypothesis is supported by IE-HPLC-UV (FIG. 9B).

FIGS. 10A-10B show that cobalt also plays an active part in the degradation process. HPLC-MS measurements were performed. ‘Standard’ ink showed a rapid 3′ONH₂-dNTP degradation profile in the presence of cobalt (FIG. 10A) and a constant [3′ONH₂-dNTP] in its absence (FIG. 10A).

FIG. 11 shows stability studies performed on ‘Standard’ ink formulated with 3′X-dNTP with different 3′ functionalization (N=A, T; X=—ONH₂ (2×P), —OCH₂N₃, —N₃, acetone oxime, —OH, —H). The aminoxy group (ONH₂) is a good nucleophile and might contribute to the formation of pyrophosphate and precipitation. Indeed, 3′ONH₂-dNTP ‘Standard’ ink precipitated fastest when stored at 20° C. followed by ‘Standard’ ink containing 3′OH-dNTP. Additionally, ‘Standard’ ink containing dTTP always precipitated faster than dATP analogues, irrespective of 3′ functionalization. We speculate that this is due to the capacity of the nucleobase to complex cobalt and participate in precipitation.

FIG. 12 shows results of separating ink components and printing them separately in the same location on storage stability and activity. To avoid printing too many inks, we chose to test only ‘two-pack’ combinations and combinations where cobalt and nucleotide are separated. Printing every component separately would take more time, require additional fluid reservoirs and printheads and introduce potential errors through imprecise alignment of the different inks at a given location. A split ink comprising 2×TdT-2×CoCl₂ (1A) and 2×Nt-2×DMSO (1B) and a split ink containing 2×TdT-2×Nt (2A) and 2×CoCl₂-2×DMSO (2B) and a split ink containing 3×Nt-2×CoCl₂-2.67×DMSO (3A) and 2.5×TdT (3B) were prepared. Inks were filtered (0.8 μm) before adding TdT and 3′-ONH₂-dNTP (N=T) and their stability at 22° C. assessed. Mixed in a 1:1 v/v ratio the split inks (parts 1A with 1B or 2A with 2B or 3A with 3B) would give ‘Standard’ or ‘Optink0.25’ ink. ‘Standard’ ink (combined ink, here called ‘Premix’) is the worst for stability, and the split ink version of ‘Standard’ ink is on a par with ‘Optink0.25’ combined and ‘Optink0.25’ split ink in terms of stability. ‘Optink0.25’ ink (combined), however, is the best ink because it can be printed in one step.

FIG. 13 shows an electrophoresis gel of a 5 cycles enzymatic DNA synthesis performed with an elongation ink formulated at a pH varying from 5.1 to 6.6 with 1 mM CoCl₂.

FIG. 14 shows an electrophoresis gel of an automated 52 cycles enzymatic DNA synthesis of Poly(T) and Sequence 1 with elongation inks formulated at active pH 5.6, 6.0 and 6.4 with 1 mM CoCl₂.

FIG. 15 shows an electrophoresis gel of a 5 cycles enzymatic DNA synthesis performed with an elongation ink formulated with varying [CoCl₂] from 1 mM to 0.1 mM.

FIG. 16 shows an electrophoresis gel of a 5 cycles enzymatic DNA synthesis performed at 20° C. with an elongation ink formulated with 1 mM [CoCl₂] or 5 mM MCl₂ (M=Mg, Mn, Ni, Zn) (condition 1) or formulated with 0.25 mM [CoCl₂] or 1.25 mM MCl₂ (M=Mg, Mn, Ni, Zn) (condition 2).

FIG. 17 shows an electrophoresis gel of a 5 cycles enzymatic DNA synthesis performed with an elongation ink formulated with varying pHs, buffers and [MgCl₂].

### DETAILED DESCRIPTION

Before the present methods, devices, and compositions are described, it is to be understood that this invention is not limited to particular methods, devices, or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a polynucleotide” includes a plurality of such polynucleotides, and reference to “the initiator” includes reference to one or more initiators and equivalents thereof, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “about”, particularly in reference to a given quantity, is meant to encompass deviations of plus or minus five percent.

The terms “polynucleotide” “oligonucleotide”, “nucleic acid”, and “nucleic acid molecule” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. More particularly, the terms “polynucleotide”, “oligonucleotide”, “nucleic acid”, and “nucleic acid molecule” include polydeoxyribonucleotides (containing 2-deoxy-D-ribose), polyribonucleotides (containing D-ribose), any other type of polynucleotide which is an N- or C-glycoside of a purine or pyrimidine base, and other polymers containing nonnucleotidic backbones, for example, polyamide (e.g., peptide nucleic acids (PNAs)) and polymorpholino (commercially available from the Anti-Virals, Inc., Corvallis, Oregon, as Neugene) polymers, and other synthetic sequence-specific nucleic acid polymers providing that the polymers contain nucleobases in a configuration which allows for base pairing and base stacking, such as is found in DNA and RNA. There is no intended distinction in length between the terms “polynucleotide”, “oligonucleotide”, “nucleic acid”, and “nucleic acid molecule,” and these terms will be used interchangeably. Thus, these terms include, for example, 3′-deoxy-2′,5′-DNA, oligodeoxyribonucleotide N3′ P5′ phosphoramidates, 2′-O-alkyl-substituted RNA, double- and single-stranded DNA, as well as double- and single-stranded RNA, DNA:RNA hybrids, and hybrids between PNAs and DNA or RNA, and also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide. In particular, DNA is deoxyribonucleic acid. Monomers making up polynucleotides and oligonucleotides are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Whenever the use of an oligonucleotide, polynucleotide, or nucleic acid requires enzymatic processing, such as extension by a polymerase, ligation by a ligase, or the like, one of ordinary skill would understand that oligonucleotides or polynucleotides in those instances would not contain certain analogs of internucleosidic linkages, sugar moieties, or bases at any or some positions. Polynucleotides typically range in size from a few monomeric units, e.g., 5-40, when they are usually referred to as “oligonucleotides,” to several thousand monomeric units. Whenever a polynucleotide, oligonucleotide, or nucleic acid is represented by a sequence of letters (upper or lower case), such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, “I” denotes deoxyinosine, “U” denotes uridine, unless otherwise indicated or obvious from context. Unless otherwise noted the terminology and atom numbering conventions will follow those disclosed in Strachan and Read, Human Molecular Genetics 2 (Wiley-Liss, New York, 1999). Usually, polynucleotides comprise the four natural nucleosides (e.g., deoxyadenosine, deoxycytidine, deoxyguanosine, deoxythymidine for DNA or their ribose counterparts for RNA) linked by phosphodiester linkages; however, they may also comprise non-natural nucleotide analogs, e.g., including modified bases, sugars, or internucleosidic linkages. It is clear to those skilled in the art that where an enzyme has specific oligonucleotide or polynucleotide substrate requirements for activity, e.g. single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al, Molecular Cloning, Second Edition (Cold Spring Harbor Laboratory, New York, 1989), and like references. Likewise, the oligonucleotide and polynucleotide may refer to either a single stranded form or a double stranded form (i.e. duplexes of an oligonucleotide or polynucleotide and its respective complement). It will be clear to one of ordinary skill which form or whether both forms are intended from the context of the terms usage.

The terms “base protecting moiety” and “base protecting group” are used interchangeably and refer to a protecting group on a nucleotide base, which may be used to reduce or eliminate the formation of secondary structure in the course of polynucleotide chain extensions and/or prevent deamination (see, e.g., International Patent Application Publication No. WO 2021/018921, herein incorporated by reference in its entirety). A base protecting group may be attached to the 6-nitrogen of deoxyadenosine triphosphate, the 2-nitrogen of deoxyguanosine triphosphate, and/or the 4-nitrogen of deoxycytidine triphosphate. In some embodiments, a base protecting group is attached to all of the indicated nitrogens. In some embodiments, a base protecting group attached to a 6-nitrogen of deoxyadenosine triphosphate is selected from the group consisting of benzoyl, phthaloyl, phenoxyacetyl, and methoxy acetyl; a base protecting group attached to the 2-nitrogen of deoxyguanosine triphosphate is selected from the group consisting of isobutyryl, isobutyryloxyethylene, acetyl, 4-isopropyl-phenoxyacetyl, phenoxyacetyl, and methoxyacetyl; and a base protecting group attached to said 4-nitrogen of deoxycytidine triphosphate is selected from the group consisting of benzoyl, phthaloyl, acetyl, and isobutyryl. In some embodiments, a protecting group attached to the 6-nitrogen of deoxyadenosine triphosphate is benzoyl; a base protecting group attached to the 2-nitrogen of deoxyguanosine triphosphate is isobutryl or dimethylformamidine; and the base protecting group attached to the 4-nitrogen of deoxycytidine triphosphate is acetyl. In some embodiments, a base protecting group attached to the 6-nitrogen of deoxyadenosine triphosphate is phenoxyacetyl; a base protecting group attached to the 2-nitrogen of deoxyguanosine triphosphate is 4-isopropyl-phenoxyacetyl or dimethylformamidine; and the base protecting group attached to the 4-nitrogen of deoxycytidine triphosphate is acetyl. In some embodiments, base protecting moieties are removed (i.e. the product is deprotected) and product is cleaved from a solid support in the same reaction. For example, an initiator may comprise a ribo-uridine which may be cleaved to release the polynucleotide product by treatment with 1 M KOH, or like reagent (ammonia, ammonium hydroxide, NaOH, or the like), which simultaneously removes base-labile base protecting moieties.

As used herein, the term “initiator”, “DNA initiator”, “initiating fragment”, “initiator nucleic acid”, “initiator oligonucleotide”, or “initiator polynucleotide” refers to an oligonucleotide or polynucleotide comprising a free 3′-hydroxyl group, which can be further elongated by a template-free polymerase (e.g., TdT).

As used herein, the term “extension product”, “extension intermediate”, elongation product” or “elongation intermediate” are used interchangeably and refer to the product resulting from enzymatic extension at the 3′ end of an initiator or of an extension intermediate generated from an initiator by a template-free polymerase.

As used herein, “inkjet assisted synthesis” means that one or more synthesis reagents are delivered to reaction sites in droplets generated by one or more inkjet printhead nozzles.

“Synthesis reagents” include any reagent used in a synthesis cycle to couple a nucleotide monomer, particularly a 3′-O-protected-nucleoside triphosphate, to an initiator or elongated fragment. Synthesis reagents may include a template-free polymerase, cofactors (e.g., Co²⁺ or other divalent cations), and nucleotides (e.g., 3′-O-protected-nucleotides), buffers, deprotection or deblocking agents, and the like.

The terms “deprotection” agent, deprotection buffer, deprotection solution, and the terms “deblocking” agent, deblocking buffer, and deblocking solution are used herein interchangeably. Likewise, the term “protected” in reference to compounds, such as, dNTPs, is used synonymously with the term “blocked” in reference to compounds.

As used herein, the term “deprotection solution” (or its equivalent terms) means a reagent that brings about or promotes the removal of a protection group, for example, a 3′-O-protecting group of a nucleotide. As described more fully below, the composition of a deprotection solution (and deprotection reaction conditions) depends on the nature of the protecting group (or blocking group) which is to be removed. In various embodiments, a deprotection solution may contain specific reagents that chemically react with a protection group and/or protected moiety (such as, a reducing agent like tris(2-carboxyethyl)phosphine (TCEP), enzymes for enzymatic cleavage, scavengers, co-factors, or the like. In some embodiments, a deprotection solution may not contain specific reagents that react with a protection group, but may contain components, e.g., pH buffers, that are compatible with or promote physical cleavage of a protecting group, such as in the case of a photocleavable protecting group. Typically, in a reaction cycle for elongating a polynucleotide fragment, in a deprotecting step a deprotection solution is incubated with 3′-O-protected elongated fragments for a predetermined incubation time. Typical incubation times (i.e. durations of incubating steps) are in the range of from 1 minute to 30 minutes; or in the range of from 3 minutes to 30 minutes; or in the range of from 3 minutes to 15 minutes. Typical elongation reaction temperatures are in the range of from room temperature (RT) to 80° C.; or from 20° C. to 80° C.; or from 20° C. to 60° C.

“Synthesis reagents” also include reagents for preparing a substrate for polynucleotide synthesis, such as, reagents for defining reaction sites, initiators, capping reagents, and the like.

“Synthesis reagents” also include reagents for preparing a substrate for polynucleotide synthesis, such as, reagents for defining reaction sites, initiators, capping reagents, and the like.

A “distinct reaction site” on a substrate is a discrete site in that it is separated from other reaction sites; that is, a discrete site does not have a border with, or overlap with, another reaction site. In other words, a discrete or different reaction site is not contiguous with, or overlapping, other reaction sites. Exceptions to this usual arrangement include “overwriting” embodiments described below for generating high density barcodes on surfaces.

A solid support is “addressable” when it has multiple features (e.g., reaction centers) positioned at particular predetermined locations (e.g., “addresses”) on the surface of the solid support.

An “array” includes any two-dimensional or substantially two-dimensional (as well as a three-dimensional) arrangement of addressable regions, e.g., spatially addressable regions. An array is “addressable” when it has multiple features (e.g., reaction centers) positioned at particular predetermined locations (e.g., “addresses”) on the array. Array features may be separated by intervening spaces.

“Primer” means an oligonucleotide, either natural or synthetic that is capable, upon forming a duplex with a polynucleotide template, of acting as a point of initiation of nucleic acid synthesis and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually, primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following references that are incorporated by reference: Dieffenbach, editor, PCR Primer: A Laboratory Manual, 2nd Edition (Cold Spring Harbor Press, New York, 2003).

“Sequence determination”, “sequencing” or “determining a nucleotide sequence” in reference to polynucleotides includes determination of partial as well as full sequence information of the polynucleotide. That is, the terms include sequences of subsets of the full set of four natural nucleotides, A, C, G and T, such as, for example, a sequence of just A's and C's of a target polynucleotide. That is, the terms include the determination of the identities, ordering, and locations of one, two, three or all of the four types of nucleotides within a target polynucleotide. In some embodiments, the terms include the determination of the identities, ordering, and locations of two, three or all of the four types of nucleotides within a target polynucleotide. In some embodiments sequence determination may be accomplished by identifying the ordering and locations of a single type of nucleotide, e.g. cytosines, within the target polynucleotide “catcgc . . . ” so that its sequence is represented as a binary code, e.g., “100101 . . . ” representing “c-(not c)(not c)c-(not c)-c . . . ” and the like. In some embodiments, the terms may also include subsequences of a target polynucleotide that serve as a fingerprint for the target polynucleotide; that is, subsequences that uniquely identify a target polynucleotide within a set of polynucleotides, e.g. all different RNA sequences expressed by a cell.

“Substantially purified” generally refers to isolation of a substance (compound, polynucleotide, protein, polypeptide, polypeptide composition) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

By “isolated” is meant, when referring to a polypeptide, that the indicated molecule is separate and discrete from the whole organism with which the molecule is found in nature or is present in the substantial absence of other biological macro-molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

“Homology” refers to the percent identity between two polynucleotide or two polypeptide moieties. Two nucleic acid, or two polypeptide sequences are “substantially homologous” to each other when the sequences exhibit at least about 50% sequence identity, preferably at least about 75% sequence identity, more preferably at least about 80%-85% sequence identity, more preferably at least about 90% sequence identity, and most preferably at least about 95%-98% sequence identity over a defined length of the molecules. As used herein, substantially homologous also refers to sequences showing complete identity to the specified sequence.

The terms “modification” or “alteration” as used herein in relation to a position or amino acid mean that the amino acid in the specific position has been modified compared to the amino acid of the wild-type protein.

A “substitution” means that an amino acid residue is replaced by another amino acid residue. For example, the term “substitution” refers to the replacement of an amino acid residue by another selected from the naturally-occurring standard 20 amino acid residues, rare naturally occurring amino acid residues (e.g. hydroxyproline, hydroxylysine, allohydroxylysine, 6-N-methylysine, N-ethylglycine, N-methylglycine, N-ethylasparagine, allo-isoleucine, N-methylisoleucine, N-methylvaline, pyroglutamine, aminobutyric acid, ornithine, norleucine, norvaline), and non-naturally occurring amino acid residue, often made synthetically, (e.g. cyclohexyl-alanine).

Amino acids may be represented by their one-letter or three-letters code according to the following nomenclature: A: alanine (Ala); C: cysteine (Cys); D: aspartic acid (Asp); E: glutamic acid (Glu); F: phenylalanine (Phe); G: glycine (Gly); H: histidine (His); I: isoleucine (Ile); K: lysine (Lys); L: leucine (Leu); M: methionine (Met); N: asparagine (Asn); P: proline (Pro); Q: glutamine (Gln); R: arginine (Arg); S: serine (Ser); T: threonine (Thr); V: valine (Val); W: tryptophan (Trp) and Y: tyrosine (Tyr).

A substitution can be a conservative or non-conservative substitution. Examples of conservative substitutions are within the groups of basic amino acids (arginine, lysine and histidine), acidic amino acids (glutamic acid and aspartic acid), polar amino acids (glutamine, asparagine and threonine), hydrophobic amino acids (methionine, leucine, isoleucine, cysteine and valine), aromatic amino acids (phenylalanine, tryptophan and tyrosine), and small amino acids (glycine, alanine and serine).

Herein, the terms “peptide”, “polypeptide”, “protein”, “enzyme”, refer to a chain of amino acids linked by peptide bonds, regardless of the number of amino acids forming said chain.

“Recombinant” as used herein to describe a nucleic acid molecule means a polynucleotide of genomic, cDNA, viral, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

The terms “connected” or “coupled” are used in an operational sense and are not necessarily limited to a direct connection or coupling. For example, two devices or components may be coupled directly, or via one or more intermediary media or devices. As another example, devices may be coupled in such a way that information or data can be passed between them, while not sharing any physical connection with one another. In some cases, two devices or components may be connected by a wire or wirelessly to each other.

### Inkjet-Assisted Enzymatic Synthesis of Polynucleotides

Methods and compositions are provided for inkjet-assisted synthesis of a plurality of polynucleotides, each at a distinct reaction site on a substrate, using template-free polymerases, such as, terminal deoxynucleotidyl transferases (TdTs). Typically, enzymatic synthesis of polynucleotides takes place on substrates comprising a planar surface, such as, glass, silica, silicon oxide, plastic, or like surfaces, but it may also take place on other surfaces, such as, for example, non-planar surfaces (e.g., beads, particles), biological tissues, or surface-immobilized cDNAs extracted from tissues. The methods disclosed herein may include the use of an inkjet printer for highly parallel template-free enzymatic synthesis of polynucleotides, as described in International Application Publication No. WO 2022/013094; herein incorporated by reference in its entirety.

In some embodiments, the plurality of polynucleotides may be in the range of from 2 to 500,000; or in the range of from 100 to 400,000; or in the range of from 100 to 200,000; or in the range of from 100 to 100,000. The plurality of polynucleotides may be the same or different than the plurality of reaction sites. In some embodiments, the plurality of reaction sites may be greater than the plurality of polynucleotides. In some embodiments, the above pluralities of reaction sites each have a density equivalent to that if uniformly deposited on an area equivalent to that of a standard 25 mm×75 mm microscope slide. In some embodiments, an array of reaction sites formed by uniform deposition may be a rectilinear array; and in other embodiments, an array of reaction sites formed by uniform deposition may be a hexagonal array.

In some embodiments, parallel synthesis is implemented by providing a support having discrete, non-overlapping, addressable sites where separate polynucleotides are synthesized and a means for controlling photoillumination, electrochemical conditions, or other reaction conditions at each site independently of the other sites. In some embodiments, such a parallel synthesis support is a planar support having a regular pattern of addressable sites, such as, a rectilinear pattern of sites, or a hexagonal pattern of sites. In some embodiments, the support is a planar support having an irregular pattern of addressable sites or complex pattern of addressable sites. In some embodiments, each site of a planar support is associated with one or more electrodes whose electrical characteristics may be controlled in an addressable manor independent of other electrodes of the planar support.

In some embodiments, the planar support comprises a plurality of sites comprising at least 256 sites, at least 512 sites, at least 1024 sites, at least 5000 sites, at least 10,000 sites, at least 25,000 sites, or at least 100,000 sites and as many as 10,000,000 sites. In some embodiments, such planar supports have a plurality of sites greater than 1000, or 10,000, or 25,000, or 50,000, or 100,000, or 500,000, and up to 1,000,000 sites or up to 10,000,000 sites, or up to 300,000,000 sites. In some embodiments, the sites of the planar support is disposed in a regular array and each site is associated with at least one electrode integrated with the planar support. In some embodiments, the discrete site at which synthesis and/or sequencing take place each has an area in the range of from 0.25 mih² to 1000 mih², or from 1 mih² to 1000 mih², or from 10 mih² to 1000 mih², or from 100 mih² to 1000 mih². In some embodiments, the amount of a polynucleotide synthesized at each site is at least 10⁻⁶ fmol, or at least 10⁻³ fmol, or at least 1 fmol, or at least 1 μmol, or the amount of polynucleotide synthesized at each site is in the range of from 10⁻⁶ fmol to 1 fmol, or from 10⁻³ fmol to 1 fmol, or from 1 fmol to 1 μmol, or from 10⁻⁶ pmol to 10 μmol, or from 10⁻⁶ pmol to 1 pmol. In some embodiments, the number of polynucleotides synthesized at each site is in the range of from 1000 molecules to 10⁶ molecules, or from 1000 molecules to 10⁹ molecules, or from 1000 molecules to 10¹² molecules.

In some embodiments, enzymatically synthesized polynucleotides at each reaction site have lengths in the range of from 50 to 1000 nucleotides.

Template-free enzymatic synthesis of polynucleotides involves cycles of steps with most involving delivery to a reaction site of at least one of the following reagents: a composition comprising a template-free polymerase, a composition comprising any cofactors needed for activity of the template-free polymerase, one or more compositions, each comprising one or more 3′-O-protected-dNTPs (i.e., monomers), a composition comprising reagents for deprotection, and wash solutions. In various embodiments, the compositions comprising the template-free polymerase, cofactors, 3′-O-protected-dNTPs, reagents for deprotection, and wash solutions may be conveyed to reaction sites by droplets created and delivered by inkjet printhead nozzles. To be delivered by inkjet-generated droplets, these reagents must be formulated to meet the rheological requirements for droplet formation. These formulations are referred to as “inks.” The key rheological parameters affecting droplet formation are viscosity, density and surface tension, e.g. Derby, Annu. Rev. Mater. Sci., 40: 395-414 (2010); Derby, J. Mater. Chem., 18: 5717-57-21 (2008); Calvert, Chem. Mater., 13: 3299-3305 (2001); Tekin et al, Soft Matter, 4: 703-713 (2008); and like references. Another key parameter relating to droplet volume is the nozzle diameters of the inkjet printhead nozzles. In some embodiments, nozzle diameters may be in the range of from 10 μm to 100 μm, including any diameter within this.

In one aspect, reagent inks are provided for inkjet-assisted enzymatic synthesis of polynucleotides, and in particular, inks comprising a template-free polymerase, particularly, inks comprising a terminal deoxynucleotidyltransferase (TdT), one or more 3′-O-protected-dNTP monomers, cofactors (e.g., divalent cations), and/or ink solvents, as described further below.

### Template-Free Enzymatic Synthesis of Polynucleotides

Enzymatic nucleic acid synthesis uses an enzymatic catalyst to carry out the polymerization of nucleotides. Enzymatic DNA synthesis is generally performed with an enzyme that catalyzes the addition of nucleotides to the 3′ end of a DNA molecule. More specifically, the process employs, without being limited thereto, enzymes which make possible the creation of a phosphodiester bond between a 3′-OH group of a nucleic acid fragment in the course of synthesis and the 5′-OH group of the nucleotide to be added during the enzymatic addition stage.

In some embodiments, enzymatic nucleic acid synthesis is performed with an enzyme capable of catalyzing the polymerization of nucleotides independently of the presence of a complementary strand (i.e., template). Such enzymes are capable of synthesizing nucleic acids in the absence of any complementary strand. In some cases, enzymatic nucleic acid synthesis may be performed with an enzyme that has the ability to synthesize single stranded nucleic acid fragments. The addition of nucleotides is thus advantageously carried out by the enzymatic route, by means of enzymes capable of polymerizing nucleotides without the presence of a template strand.

In some embodiments, the enzyme chosen for use in enzymatic nucleic acid synthesis is a template-free polymerase selected from translesion DNA polymerases of type η (eta) or ζ (zeta), polynucleotide phosphorylases (PNPases), template-independent RNA polymerases, terminal transferases, template-independent DNA polymerases, reverse transcriptases, 9° N DNA polymerases, or terminal deoxynucleotidyl transferases (TdT). These enzymes are expressed by certain cells of living organisms and can be extracted from these cells or purified from recombinant cultures.

In some embodiments, an engineered terminal deoxynucleotidyl transferase is used to perform enzymatic nucleic acid synthesis. Various variants of terminal deoxynucleotidyl transferase have been developed for this purpose. See, e.g., U.S. Pat. Nos. 11,208,637; 10,752,887; 10,435,676; and U.S. Patent Application Publication No. 2022/0002687; herein incorporated by reference in their entireties. In some embodiments, an engineered reverse transcriptase is used to perform enzymatic nucleic acid synthesis. For example, human immunodeficiency virus type-1 and Moloney murine leukemia virus reverse transcriptases may be used. Engineered Moloney murine leukemia virus reverse transcriptase variants are commercially available such as the SuperScript IV reverse transcriptase from Thermo Fisher (Waltham, MA) and SMARTScribe reverse transcriptase from Clonetech (Mountain View, Calif.).

In some embodiments, an engineered 9° N DNA polymerase is used to perform enzymatic DNA synthesis. Engineered 9° N DNA polymerase variants are commercially available, including duplases from Centrillion Technology Holdings Corporation (Grand Cayman, KY) and the Therminator Thermococcus sp. DNA polymerase from New England Biolabs (Ipswich, MA). See also, e.g., Hoff et al. (2020) ACS Synth Biol 9(2):283-293; Gardner et al. (2019) Front. Mol. Biosci. 6:28; herein incorporated by reference in their entireties.

A cycle of the enzymatic synthesis process, leading to the addition of a nucleotide to a nucleic acid strand, comprises two successive steps, an elongation step and a deprotecting step respectively. During the elongation step, the polymerase adds a nucleotide comprising a protecting group to a nucleic acid strand. Then the protection group is removed from this newly added nucleotide, to be able to perform additional cycles.

Synthesis of a complete nucleic acid by template-free enzymatic nucleic acid synthesis typically comprises repeated cycles of steps, in which a selected nucleotide is coupled to an initiator or growing chain in each cycle.

As used herein, an “initiator” (or equivalent terms, such as, “initiating fragment”, “initiator nucleic acid”, “initiator oligonucleotide”, “initiator polynucleotide”, or the like) refers to an oligonucleotide or polynucleotide comprising a free 3′-hydroxyl group, which can be further elongated by a template-free polymerase (e.g., TdT). In one embodiment, the initiator is a DNA initiating fragment. In an alternative embodiment, the initiator is an RNA initiating fragment. In some embodiments, an initiator comprises between 3 and 100 nucleotides. In some embodiments, an initiator comprises between 3 and 20 nucleotides. In some embodiments, the initiator is single-stranded. In alternative embodiments, the initiator is double-stranded. In some embodiments, an initiator may comprise a non-nucleic acid compound having a free hydroxyl group to which a TdT may couple a 3′-O-protected dNTP (see, e.g., Baiga, U.S. patent publications US2019/0078065 and US2019/0078126; herein incorporated by reference). The general elements of template-free enzymatic synthesis are described in the following references: Ybert et al, International patent publication WO/2015/159023; Ybert et al, International patent publication WO/2017/216472; Hyman, U.S. Pat. No. 5,436,143; Hiatt et al, U.S. Pat. No. 5,763,594; Jensen et al, Biochemistry, 57: 1821-1832 (2018); Mathews et al, Organic & Biomolecular Chemistry, DOI: 0.1039/c6ob01371f (2016); Schmitz et al, Organic Lett., 1(11): 1729-1731 (1999). In some embodiments, an initiator oligonucleotide may be attached to a synthesis support by its 5′end; and in other embodiments, an initiator oligonucleotide may be attached indirectly to a synthesis support by forming a duplex with a complementary oligonucleotide that is directly attached to the synthesis support, e.g. through a covalent bond. In some embodiments a synthesis support is a solid support which may be a discrete region of a solid planar solid, or may be a bead.

Initiators are provided, for example, attached to a solid support, with a free 3′-hydroxyl groups. To the initiator (or elongated initiator polynucleotides in subsequent cycles) are added a 3′-O-protected-dNTP and a template-free polymerase, such as a TdT or a variant thereof (e.g., Ybert et al, WO/2017/216472) under conditions effective for the enzymatic incorporation of the 3′-O-protected-dNTP onto the 3′-end of the initiator (or elongated initiator polynucleotides). This reaction produces elongated initiator polynucleotides whose 3′-hydroxyls are protected. If the elongated initiator polynucleotide contains a competed sequence, then the 3′-O-protection group may be removed, or deprotected, and the desired sequence may be cleaved from the original initiator. Such cleavage may be carried out using any of a variety of single strand cleavage techniques, for example, by inserting a cleavable nucleotide at a predetermined location within the original initiator. An exemplary cleavable nucleotide may be a uracil nucleotide which is cleaved by uracil DNA glycosylase. If the elongated initiator polynucleotide does not contain a completed sequence, then the 3′-O-protection groups are removed to expose free 3′-hydroxyls and the elongated initiator polynucleotides are subjected to another cycle of nucleotide addition and deprotection. In some embodiments, 3′-O-protection groups are electrochemically labile groups. That is, deprotection or cleavage of the protection group is accomplished by changing the electrochemical conditions in the vicinity of the protection group which result in cleavage. Such changes in electrochemical conditions may be brought about by changing or applying a physical quantity, such as a voltage difference or light to activate auxiliary species which, in turn, cause changes in the electrochemical conditions at the site of the protection group, such as an increase or decrease in pH. In some embodiments, electrochemically labile groups include, for example, pH-sensitive protection groups that are cleaved whenever the pH is changed to a predetermined value. In other embodiments, electrochemically labile groups include protecting groups which are cleaved directly whenever reducing or oxidizing conditions are changed, for example, by increasing or decreasing a voltage difference at the site of the protection group.

In some embodiments, an ordered sequence of nucleotides are coupled to an initiator using a template-free polymerase, such as TdT, in the presence of 3′-O-protected dNTPs at each synthesis step. In some embodiments, the method of synthesizing an oligonucleotide comprises the steps of (a) providing an initiator having a free 3′-hydroxyl; (b) reacting under extension conditions the initiator or an extension intermediate having a free 3′-hydroxyl with a template-free polymerase in the presence of a 3′-O-protected nucleoside triphosphate to produce a 3′-O-protected extension intermediate; (c) deprotecting the extension intermediate to produce an extension intermediate with a free 3′-hydroxyl group; and (d) repeating steps (b) and (c) until the polynucleotide is synthesized. (The terms “extension intermediate,” “extension product” and “elongation fragment” are used interchangeably). In some embodiments, an initiator is provided as an oligonucleotide attached to a solid support, e.g., by its 5′ end. The above method may also include washing steps after the reaction, or extension, step, as well as after the de-protecting step. For example, the step of reacting may include a sub-step of removing unincorporated nucleoside triphosphates, e.g., by washing, after a predetermined incubation period, or reaction time. Such predetermined incubation periods or reaction times may be a few seconds, e.g., 30 seconds, to several minutes, e.g., 30 minutes.

The 3′-O-blocked dNTPs employed may be purchased from commercial vendors or synthesized using published techniques (see, e.g., U.S. Pat. No. 7,057,026; Guo et al, Proc. Natl. Acad. Sci., 105(27): 9145-9150 (2008); Benner, U.S. Pat. No. 7,544,794; herein incorporated by reference in their entireties.

The above method may also include capping step(s) as well as washing steps after the reacting, or extending, step, as well as after the deprotecting step. As mentioned above, in some embodiments, capping steps may be included in which non-extended free 3′-hydroxyl groups are reacted with compounds that prevents any further extension of the capped strand. In some embodiments, the compound is a dideoxynucleoside triphosphate. In other embodiments, non-extended strands with free 3′-hydroxyl groups are degraded by treating them with a 3′-exonuclease activity, e.g., Exo I. For example, see Hyman, U.S. Pat. No. 5,436,143. Likewise, in some embodiments, strands that fail to be deblocked may be treated to either remove the strand or render it inert to further extensions.

### Inks for Inkjet-Assisted Enzymatic Synthesis of Polynucleotides

Synthesis reagents delivered by inkjet printhead nozzles must be formulated so as to preserve activity of synthesis reagents, avoid formation of precipitates that clog the printhead nozzle, and meet the rheological requirements for droplet formation. Printable reagent compositions are referred to herein as “inks.”

For example, satisfying the first constraint (activity) may require that a template-free polymerase be present in a reaction mixture at a certain minimal concentration. However, because of high protein viscosity, the concentration for the desired activity may interfere with the second constraint, i.e., that the reagent composition be capable of droplet formation. In some embodiments the polymerase may be delivered in a plurality of droplets, each with lower concentrations of polymerase, which coupled with evaporation permit the build-up of the polymerase concentration to provide a desired level of activity.

In some embodiments, inks comprise combinations of premixed synthesis reagents. Preferred combinations of synthesis reagents retain enzymatic activity without formation of precipitates under operating conditions for inkjet-assisted synthesis and remain stable during storage. In some embodiments, an ink is stable (i.e., retains enzymatic activity without formation of detectable precipitates) for at least 3 days, at least 4 days, at least 5 days, at least 10 days, at least 20 days, or at least 30 days, or longer. Preferably, the ink remains stable for at least the length of time required to synthesize a polynucleotide of interest. In some embodiments, sets of printable reagent compositions or “inks” are used in inkjet-assisted synthesis.

In some embodiments, a printable reagent composition is provided, the printable reagent composition comprising: a template-free polymerase, a 3′-O-protected nucleoside triphosphate, a divalent cation, wherein the divalent cation is a cobalt divalent cation (Co²⁺) and the divalent cation concentration is ranging from 0.1 mM to 1 mM, preferably from 0.2 to 0.5 mM, even more preferably is about 0.25 mM and the pH of the printable reagent composition is ranging from pH 5.1 to 6.6, preferably from pH 5.6 to 6.6, preferably from pH 5.6 to 6.5, more preferably from pH 5.6 to 6.4, even more preferably from pH 6.0 to 6.4, even more preferably the pH is about 6.0, or
wherein the divalent cation is a manganese divalent cation (Mn²⁺) and the divalent cation concentration is ranging from 0.5 mM to 7 mM, preferably from 1 to 6 mM, even more preferably is about 1.25 mM and the pH of the printable reagent composition is ranging from pH 6.0 to 7.0, preferably from pH 6.4 to 6.8, more preferably the pH is about 6.6, or
wherein the divalent cation is magnesium divalent cation (Mg²⁺) and the divalent cation concentration is ranging from 1 mM to 20 mM, preferably from 2 to 15 mM, preferably from 3 to 8 mM, even more preferably is about 5 mM and the pH of the printable reagent composition is ranging from pH 5.6 to 8.4, preferably from pH 7.0 to 8.2, more preferably the pH is about 8.0.

In an alternative embodiment the divalent cation has a concentration ranging from 0.1 mM to 5.0 mM, a buffer, the printable reagent composition has a from pH 5.1 to pH 6.6, or from pH 5.1 to pH 6.5, or from pH 5.1 to pH 6.4, or from pH 5.6 to pH 6.6, or from pH 5.6 to pH 6.5, or from pH 5.6 to pH 6.4, or from pH 6.0 to pH 6.6, or from pH 6.0 to pH 6.5, or from pH 6.0 to pH 6.4, or from pH 6.0 to pH 6.2, or from pH 5.1 to pH 7.2, or from pH 5.1 to pH 8.4, and the divalent cation is a cobalt divalent cation (Co²⁺), a manganese divalent cation (Mn²⁺), a zinc divalent cation (Zn²⁺), a nickel divalent cation (Ni²⁺), or a magnesium divalent cation (Mg²⁺), preferably the divalent cation is a cobalt divalent cation (Co²⁺), a manganese divalent cation (Mn²⁺), or a magnesium divalent cation (Mg²⁺), more preferably a cobalt divalent cation (Co²⁺).

In some embodiments, the printable reagent composition is split into a first reagent composition comprising the template-free polymerase and the divalent cation; and a second reagent composition comprising the 3′-O-protected nucleoside triphosphate and the polar organic solvent.

In other embodiments, the printable reagent composition is split into a first reagent composition comprising the template-free polymerase and the 3′-O-protected nucleoside triphosphate; and a second reagent composition comprising the divalent cation, and the polar organic solvent.

In some embodiments, the 3′-O-protected nucleoside triphosphate has a concentration ranging from 100 μM to 2000 μM, including any concentration within this range.

In some embodiments, the template-free polymerase has a concentration ranging from 5 μM to 30 μM, including any concentration within this range.

In some embodiments, the polar organic solvent is dimethyl sulfoxide (DMSO), a betaine, or an alcohol such as methanol or ethanol. In some embodiments, the polar organic solvent has a concentration ranging from 5 volume/volume percent to 30 volume/volume percent, including any concentration within this range such as 5, 6, 7, 8, 9, 10, 11, 12, 13, 14, 15, 16, 17, 18, 19, 20, 21, 22, 23, 24, 25, 26, 27, 28, 29, or 30 volume/volume percent. In some embodiments, the polar organic solvent reduces DNA melting temperature.

The reagent composition preferably does not contain any exogenous source of pyrophosphate. In some embodiments, an inorganic pyrophosphatase is added to a reagent composition to remove any contaminating pyrophosphate and prevent precipitation resulting from complexation of pyrophosphate with divalent cations (e.g., Co²⁺) and/or other ligands. Inorganic pyrophosphatase catalyzes the hydrolysis of pyrophosphate to produce two phosphate ions. Any suitable soluble prokaryotic or eukaryotic inorganic pyrophosphatase may be used. A number of inorganic pyrophosphatases are commercially available from various companies, including, but not limited to, Thermo Fisher Scientific (Waltham, MA), New England Biolabs (Ipswich, MA), and Sigma-Aldrich (St. Louis, MO).

In some embodiments, a reagent composition is purified to remove contaminating pyrophosphate. Pyrophosphate can be removed, for example, using anion exchange chromatography, reverse phase chromatography, or any other suitable technique known in the art. In particular, nucleotide reagents may be contaminated with pyrophosphate and can be purified to remove any contaminating pyrophosphate prior to mixing with the other reagents of the printable reagent composition.

In some embodiments, a printable reagent composition may comprise more than one 3′-O-protected nucleoside triphosphate. In some embodiments, a printable reagent composition may comprise all four monomer types, including a 3′-O-protected-deoxyadenosine triphosphate, a 3′-O-protected-deoxythymidine triphosphate, a 3′-O-protected-deoxycytidine triphosphate, and a 3′-O-protected-deoxyguanosine triphosphate for the purpose of synthesizing random sequence segments of polynucleotides, for example, for the creation of oligonucleotide tags or barcodes.

Reagent compositions may be filtered through one or more filters prior to dispensing to the reaction sites. To avoid adsorption of the template-free polymerase to the filter, preferably a low protein binding filter is used that does not absorb the enzyme or reduce enzymatic activity. Exemplary low protein binding filters include but are not limited to, polytetrafluoroethylene (PTFE) filters, cellulose acetate filters, glass fiber filters, polyethersulfone (PES) filters, polypropylene (PP) filters, polyvinylidene fluoride (PVDF) filters, regenerated cellulose (RC) filters, and Anopore™ inorganic membrane filters.

In some embodiments, the printable reagent composition is filtered through at least one filter having a pore size ranging from 0.6 μm to 5 μm in diameter, including any pore size within this range. In some embodiments, the printable reagent composition is filtered through at least one filter, at least two filters, at least three filters, or more. The filters may have the same size or different sizes. In some embodiments, the printable reagent composition is filtered at least one time, at least two times, at least three times, or at least four times, or more. In some embodiments, the printable reagent composition is filtered through at least one filter having a pore size less than or equal to 0.8 μm in diameter.

In some embodiments, some reagents are prefiltered before mixing with other reagents to produce the printable reagent composition. In an exemplary embodiment, the printable reagent composition is prepared by a method comprising: mixing the divalent cation, the polar organic solvent, and the buffer to form a solution; filtering the solution through a first filter to form a filtered solution; adding the template-free polymerase and the 3′-O-protected nucleoside triphosphate to the filtered solution to form the final printable reagent composition; and filtering the printable reagent composition through a second filter before dispensing. In some embodiments, the first filter has a pore size of 0.8 μm in diameter, and the second filter has a pore size of 5 μm in diameter.

In some embodiments, the 3′-O-protected nucleoside triphosphate has been double or triple purified prior to its addition to prepare the printable reagent composition.

In some embodiments, printable reagent compositions are delivered in droplets ranging in volume from 1 pL to 200 pL, or from 1 pL to 100 pL, or from 1 pL to 50 pL, or preferably from 1 pL to 30 pL. Any volume in these ranges can be delivered in droplets as well.

In some embodiments, a printable reagent composition is dispensed by an inkjet at a temperature above 18° C. For example, printable reagent compositions may be dispensed by an inkjet printer at a temperature ranging from 19° C. to 45° C., including any temperature within this range. In some embodiments, the dispensing of a printable reagent composition is performed at room temperature.

In some cases, reagent compositions may be stored before use in inkjet-assisted synthesis. In some embodiments, printable reagent compositions are stored for 1 day, 2 days, 3 days, 4 days, 5 days, 6 days, 1 week, 2 weeks, 3 weeks, 1 month, 2 months, or longer before use in inkjet-assisted synthesis. Reagent compositions may be stored, for example, at temperatures ranging from about −20° C. to room temperature. In some embodiments, the printable reagent composition is stored at a temperature in a range from −20° C. to 4° C. prior to said dispensing, including any temperature within this range. In some embodiments, printable reagent compositions are made shortly before performing inkjet-assisted synthesis.

As also mentioned above, the key solution parameters affecting droplet formation by inkjets are viscosity, surface tension, density and diameter of the inkjet nozzle, which are related through the formula: Z=[(ργa)\hat{ }0.5)]/η, where ρ is the density of the fluid, γ is surface tension, η is viscosity, a is the radius of the inkjet printhead nozzle and Z is in the range of from 1 to 10 for reliable droplet formation, e.g. Derby, J. Mater. Chem., 18: 5717-5721 (2008). This relationship applies to any of the printable reagent compositions, described herein, that are delivered by inkjet-generated droplets. Applying this relationship to determine ink compositions that are capable of forming desired droplets for particular embodiments may be carried out by one of ordinary skill in the art by adjusting densities of reactants, viscosity modifiers, surface tension modifiers, and the like.

Thus, in some embodiments, printable reagent compositions further comprise viscosity modifiers, surface tension modifiers and density modifiers, and the like, in order to form “printable inks” that may be delivered in droplets generated by inkjet printhead nozzles. “Printable” in reference to a reagent ink means repeatable droplets are able to be ejected from the nozzle, with uniform velocities and volumes and no satellite droplets.

In some embodiments, the viscosity of a printable reagent composition is less than 8 cP when measured at 20° C. In some embodiments, the viscosity of the printable reagent composition ranges from 1 cP to 8 cP, more preferably from 2 cP to 8 cP when measured at 20° C. In some embodiments, the viscosity of the printable reagent composition ranges from 1 cP, preferably from 2 cP, to 7 cP, including any viscosity within this range, when measured at 20° C. In some embodiments, the viscosity of the printable reagent composition ranges from 1 cP, preferably from 2 cP, to 3 cP when measured at 20° C.

In some embodiments, a printable reagent composition comprises a viscosity modifying agent. Suitable modifying agents include, but are not limited to, glycerol, glycerol acetate, ethylene glycol, polyethylene glycol of different molecular weights, poly(vinyl alcohol), carboxymethyl cellulose and hydroxyethyl cellulose. In some embodiments, said viscosity modifier is preferably chosen from the group consisting in DMSO, PEG, t-butanol and at least one of their combinations.

In some embodiments, a printable reagent composition comprises a surface tension modifier. In some cases, the surface tension modifier may be a detergent. Suitable detergents include, but are not limited to, Tween 20, Triton X-100, CHAPS, NP-40, octyl thioglucoside, octyl glucoside and dodecyl maltoside. Additional surface tension modifiers (i.e., surfactants) are disclosed in Buret, LabChip, 12: 422-433 (2012). In some embodiments, printable printable reagent compositions are formulated as emulsions. In some embodiments, printable reagent compositions comprising TdT are formulated as emulsions.

In some embodiments, a printable reagent composition comprises a humectant for reducing droplet evaporation. Suitable humectants include, but are not limited to, glycerol, glycerol acetate, alcohol sugars, ethylhexylglycerin, panthenol, sorbitol, xylitol, maltitol, propylene glycol, hexylene glycol, butylene glycol, sodium lactate, hyaluronic acid, and polydextrose,

In some embodiments, a printable reagent composition further comprises a buffer suitable for enzymatic activity of a template-free polymerase. Any suitable buffer that can maintain the appropriate pH may be used. Exemplary buffers include cacodylate, HEPES, MES, Tris, imidazole, ADA, ACES, PIPES, MOPS, MOPSO, BES, TES, DIPSO, MOBS, TAPSO, HEPPSO, POPSO, TEA, EPPS, Tricine, and Bicine. In some embodiments, the buffer comprises about 10 to about 500 mM potassium cacodylate, MES, or HEPES. Buffer components preferably lack free hydroxyl groups and cannot complex the divalent cation (e.g., Co²⁺) used as a cofactor for the template-free polymerase to promote activity and maintain stability of the enzyme.

Preferably, any buffer, non-ionic surfactant, viscosity modifying agent, surface tension modifying agent, or aldehyde scavenger present in the printable reagent composition does not inhibit the template-free polymerase or have a free hydroxyl group that can serve as a substrate for the template-free polymerase. Any component of the reagent composition, which can act as a substrate for the template-free polymerase, can potentially interfere with DNA extension by competitive inhibition or by generating pyrophosphate in situ, which increases the reverse reaction. Preferably all potential sources of pyrophosphate and free hydroxyl groups (except the 3′-hydroxyl groups of the nucleotides, initiator, and DNA) are eliminated from the reagent composition.

In some embodiments, if the specific activity of a template-free polymerase is relatively low, so that a relatively large amount of protein must be delivered to reaction sites to complete a coupling step, then the delivery of the polymerase may be carried out by dispensing a plurality of droplets in each coupling cycle together with allowing a controlled amount of evaporation to maintain a reaction volume within a prescribed range, e.g. 10-100 pL. In some embodiments, the plurality of droplets delivered is in the range of from 2 to 10, or in the range of from 2 to 5, or in the range of from 2 to 3. In other embodiments, the plurality of droplets may be in the range of from 2 to 150, or in the range of from 10 to 120. In some embodiments, whenever the template-free polymerase is a TdT, the plurality of droplets is the number required to bring the concentration of TdT in the reaction mixture at a reaction site to a value in the range of from 5 μM to 30 μM. In some embodiments, a concentration of TdT in an ink is the concentration that produces an approximate 1:1 stoichiometry between TdT molecules and polynucleotides at a reaction site. In other embodiments, a concentration of TdT in an ink is a concentration that produces a stoichiometry between TdT molecules and polynucleotides at a reaction site that is 1:1 or greater.

In some embodiments, a printable ink comprises a TdT variant and a viscosity modifier. In some embodiments, the TdT is in a concentration in the range of 5 μM to 30 μM in a buffer suitable for coupling activity. In some embodiments, such buffer comprises about 10 to about 500 mM potassium cacodylate buffer. In some embodiments, TdT inks are characterized by a viscosity of about 1 centipoise (0.001 Pa-sec) to about 20 centipoise (0.02 Pa-sec) when viscosity is measured at room temperature; and a surface tension of about 15 dynes/cm and about 50 dynes/cm when measured at room temperature.

In some embodiments, the viscosity modifier is selected from the group consisting of glycerol, glycerol acetate, ethylene glycol, polyethylene glycol of different molecular weights, poly(vinyl alcohol), carboxymethyl cellulose and hydroxyethyl cellulose.

In some embodiments, a template-free polymerase ink, such as a TdT ink, comprises glycerol or glycerol acetate at a concentration in the range of from 5 percent (w/w) to 55 percent (w/w); in other embodiments, a template-free polymerase ink, such as a TdT ink, comprises glycerol or glycerol acetate at a concentration in the range of from 5 percent (w/w) to 40 percent (w/w); in other embodiments, a template-free polymerase ink, such as a TdT ink, comprises glycerol or glycerol acetate at a concentration in the range of from 5 percent (w/w) to 25 percent (w/w); in other embodiments, a TdT ink comprises glycerol or glycerol acetate at a concentration in the range of from 8 percent (w/w) to 40 percent (w/w); in other embodiments, a TdT ink comprises glycerol or glycerol acetate at a concentration in the range of from 8 percent (w/w) to 25 percent (w/w); in other embodiments, a TdT ink comprises glycerol or glycerol acetate at a concentration in the range of from 10 percent (w/w) to 40 percent (w/w). It is understood by those of ordinary skill that the viscosity ranges achieved by the above glycerol or glycerol acetate concentration ranges also may be achieved by equivalent concentration ranges of other viscosity modifiers. Thus, in some embodiments, a TdT ink comprises a concentration of a viscosity modifier that produces an equivalent viscosity as glycerol or glycerol acetate at a concentration in the range of from 10 percent (W/W) to 40 percent (w/w). In addition to glycerol or glycerol acetate, of particular interest are the viscosity modifiers carboxymethyl cellulose and hydroxyethyl cellulose which have minimal effect on template-free polymerase coupling activity, such as TdT coupling activity. In some embodiments, besides a viscosity modifier, the printable template-free polymerase ink, such as a TdT ink, comprises a surface tension modifier. Such surface tension modifier may be a detergent. Such detergent may be selected from Tween 20, Triton X-100, CHAPS, NP-40, octyl thioglucoside, octyl glucoside or dodecyl maltoside. Of particular interest is Triton X-100. Also of particular interest is Tween 20.

In some embodiments, said viscosity modifier is preferably chosen from the group consisting in DMSO, PEG, t-butanol and at least one of their combinations. In some embodiments, the viscosity modifier is at a concentration in the range of from 5 percent (w/w) to 55 percent (w/w), preferably in the range of from 10 percent (w/w) to 20 percent (w/w).

In cases where 3′-O-amino-dNTP monomers are employed, the presence of an aldehyde scavenger in the inks reduces spurious capping of the 3′-amines by reaction with adventitious aldehydes or ketones, such as formaldehyde, which are pervasive in the environment. This is a special problem with inkjet synthesis because droplets of ink have very high surface-to-volume ratios that enhances absorption of environmental aldehydes. Thus, in embodiments employing 3′-O-amino-dNTP monomers, inks as described above further include an effective amount of at least one aldehyde scavenger. As used herein, “effective amount” in reference to an aldehyde scavenger means an amount (or concentration) sufficient to produce a measurable decrease in spuriously capped polynucleotides in a product. Such measurements may be made readily using conventional techniques, e.g., DNA sequence analysis of a sample of a product, gel electrophoresis, or the like. As used herein, the term “aldehyde scavenger” includes ketone scavengers. In some embodiments, aldehyde scavengers are agents that react with compounds having chemical groups of the formula R—C(═O)H or R¹—C(═O)—R², where R, R¹ and R² are typically alkyl or aryl. More particularly, in some embodiments, aldehyde scavengers are agents that react with R—C(═O)H or R¹—C(═O)—R² groups on compounds at a sufficiently high rate that such compounds do not react with (or react only negligibly with) the 3′-amine group of 3′-O-amino-nucleotides. As used herein, the term “scavenger” means a chemical substance added to a mixture in order to remove or deactivate impurities or compounds that lead unwanted reaction products. In various embodiments, aldehyde scavengers may be in solution, immobilized on the materials used for storage or synthesis or coupled to reagents employed in method of the invention, for example, template-free polymerases, such as TdTs.

As noted above, enzymatic synthesis may be carried out using a variety of reagents (referred to herein as “synthesis reagents”) that may contain or consist of reactants, wash solutions, deprotection buffers, enzymes, and the like. (The term “synthesis reagent” means any reagent used in a synthesis cycle to couple a monomer, particularly a 3′-O-amino-nucleoside triphosphate, to an initiator or elongated fragment, such as, buffers comprising a template-free polymerase, buffers comprising 3′-O-protected-nucleotide monomers, deprotection (or deblocking) buffers, and the like.) In various embodiments, an aldehyde scavenger may be a component of one or more of the synthesis reagents. In some embodiments, an aldehyde scavenger may be added to a reaction mixture as a separate synthesis reagent (without other reactants, wash buffers or enzymes). In some embodiments, an aldehyde scavenger is added to a reaction mixture as a component of a synthesis reagent comprising a template-free polymerase.

In some embodiments, an ink comprises an aldehyde scavenger at a concentration in the range of from 1 to 500 mM, or in other embodiments in the range of from 1 to 200 mM, or in other embodiments in the range of from 1 to 100 mM.

Exemplary aldehyde scavengers are disclosed by Sudo et al, U.S. patent publication US2020/0061225, herein incorporated by reference. In some embodiments, the aldehyde scavengers employed comprise O-substituted hydroxylamines or polyhydroxylamines. In some embodiments, O-substituted hydroxylamines used in the subject methods are defined by the formula:

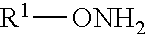

such as disclosed by Sudo et al, U.S. patent publication US2020/0061225, or Kitasaka et al, U.S. Pat. No. 7,241,625, which are incorporated herein by reference. In some embodiments, R¹ is a C₁₋₁₈ linear, branched or cyclic alkyl group which may be substituted by at least one substituent selected from the group consisting of a halogen atom; a C₁₋₆ alkyloxy group; a C₁₋₆ haloalkyl group; a C₁₋₆ haloalkyloxy group; a carboxy group; a hydroxy group; a mercapto group; a cyano group; a nitro group; a C₆₋₁₄ aryl group which may be substituted by a halogen atom, a C₁₋₆ alkyl group, a C₁₋₆ alkyloxy group, a C₁₋₆ haloalkyl group, a C₁₋₆ haloalkyloxy group, a carboxy group, a hydroxy group, a mercapto group, a cyano group or a nitro group; a C₄₋₁₄ heteroaryl group which may be substituted by a halogen atom, a C₁₋₆ alkyl group, a C₁₋₆ alkyloxy group, a C₁₋₆ haloalkyl group, a C₁₋₆ haloalkyloxy group, a carboxy group, a hydroxy group, a mercapto group, a cyano group or a nitro group; an alkoxycarbonyl group represented by the following formula:

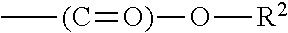

and a carbamoyl group represented by the following formula:

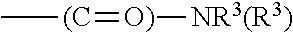

wherein R² is a C₁₋₁₈ linear, branched or cyclic alkyl group which may be substituted, at a chemically acceptable optional position, by at least one substituent selected from the group consisting of a carboxy group; a hydroxy group; a mercapto group; a halogen atom; a C₁₋₆ alkyloxy group; a C₁₋₆ haloalkyloxy group; a C₆₋₁₄ aryl group; and a C₄₋₁₄ heteroaryl group; and wherein each R³ may be the same or different and each independently a C₁₋₁₈ linear, branched or cyclic alkyl group which may be substituted by at least one substituent selected from the group consisting of a carboxy group; a hydroxy group; a mercapto group; a halogen atom; a C₁₋₆ alkyloxy group; a C₁₋₆ haloalkyloxy group; a C₆₋₁₄ aryl group; and a C₄₋₁₄ heteroaryl group; a C₆₋₁₄ aryl group, a C₄₋₁₄ heteroaryl group, or a hydrogen atom.

In some embodiments, the aldehyde scavengers comprise carbonyl compounds disclosed by Pacifici, U.S. Pat. No. 5,446,195 or Burdeniuc et al, U.S. patent publication, US20160369035; which are incorporated herein by reference, and are defined by the formula:

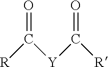

wherein R and R′ are CH₃ or H[O(CH₂)m]nO— and wherein m and n are selected from the group of combinations of m and n consisting of: m=1 and n=1, 3-19; m=2 and n=2-19; or m=3 and n=1-19, Y is —CH₂— or —CH₂—CO—CH₂—.

In some embodiments, inks as described above further include a dye to permit monitoring of the location, size, shape and possible overlap of reaction sites, either at an initial dispensing of reagents to define the reaction sites or at subsequent droplet dispensations during synthesis, particularly to monitor possible coalescence of reaction mixtures at adjacent sites. A large selection of fluorescent and non-fluorescent dyes are available for this purpose. The main criteria for use is that the dye (i) not adversely affect the performance of any reaction component, (ii) be bright or concentrated enough to make droplets or reaction sites readily detectable, (iii) be spectrally distinct if more than one is used, and (iv) not affect the rheological properties of the ink. In some embodiments, food dyes are used in inks of the invention. In other embodiments, pH indicator dyes are used in inks of the invention. In other embodiments, fluorescent dyes are used in inks of the invention. Exemplary dyes for use with inks include Brilliant Blue FCF, Fast Green FCF, Ponceau 4R and Sunset Yellow FCF. In some embodiments, food dyes are used at a concentration in the range of from 1 to 20 mM, or at a concentration in the range of from 1 to 10 mM.

### Delivery of Inks Using an Inkjet

Inkjet assisted enzymatic synthesis of polynucleotides may be implemented in a variety of embodiments in which a set of printable ink reagent compositions is delivered by inkjet printhead nozzles. In some embodiments, the surface of a reaction site comprises a layer of initiator oligonucleotides and is surrounded by a hydrophobic surface of substrate, which allows the reaction site to be enveloped by a volume of aqueous liquid on the surface without spreading or coalescing with liquid from another reaction site. The pintable reagent composition ink, as described above, is dispensed as droplets at a reaction site via an inkjet printhead nozzle. The pintable reagent composition ink comprises predetermined concentrations of template-independent polymerases, divalent cations, and nucleotides, and may, in addition, include salts and buffer components for polymerase activity and viscosity modifiers and surface tension modifiers as needed to meet the rheological requirements for droplet formation. Droplets may also include humectants to minimize evaporation loss. In some embodiments, droplets may further include an aldehyde scavenger whenever 3′-O-amino-NTPs are employed. A droplet of each ink of a set may be deposited on a dried reaction site or coalesce with a volume of liquid on an undried reaction site to form a reaction mixture, which is allowed to incubate for a predetermined time to permit coupling of nucleotide monomers to the 3′ ends of the initiators (or previously extended or elongated strands after the initial cycle). In some embodiments, such incubation takes place at a higher than ambient humidity to prevent drying during the incubation step. In some embodiments, a separate step of drying reaction sites is implemented to prevent fluid accumulation and/or coalescence with reaction mixtures at adjacent reaction sites. After the incubation time for the coupling reaction has elapsed, the entire substrate surface may be immersed in or sprayed with a deprotection buffer for a predetermined time to permit removal of a protection group, which regenerates free 3′-hydroxyls at the ends of the elongated strands. Alternatively, a deprotection buffer may be delivered to the reaction sites by an inkjet printhead nozzle. After the predetermined deprotection time has elapsed, the entire substrate surface is immersed one or more times in one or more wash buffers for predetermined times to give reaction sites with extended or elongated strands or fragments, which are ready for the next coupling cycle. In some embodiments, as mentioned above, a drying step may be implemented after deprotection and washing in order to minimize the chance of droplet spreading or coalescing with adjacent droplets. Conventional drying techniques in inkjet printing may be used, warm air or gas, radiative drying, or the like, e.g., Hoynant et al, U.S. Pat. No. 8,485,096. Depending on the nature of substrate, a coupling cycle may also include a drying step so as to prevent droplet spreading and coalescence between adjacent reaction sites. If the surface of the substrate between reaction sites is sufficiently hydrophobic, the possibility of such coalescence is minimized. Synthesis cycles are repeated until synthesis of the plurality of polynucleotides is completed at the reaction sites.

In some embodiments, the method of enzymatically synthesizing a plurality of polynucleotides each having a predetermined sequence at reaction sites on a substrate comprises: (a) providing the substrate, wherein the substrate comprises initiators at a plurality of reaction sites, wherein each initiator has a free 3′-hydroxyl group, and wherein each polynucleotide of the plurality is assigned to a reaction site for synthesis; (b) providing a printable reagent composition comprising: a template-free polymerase, a divalent cation, a 3′-O-protected nucleoside triphosphate, and a polar organic solvent that reduces DNA melting temperature; (c) performing a reaction cycle comprising the steps of i) dispensing through one or more inkjet printhead nozzles at least one droplet of the printable reagent composition to each reaction site of the plurality, wherein the initiator or elongated fragments having free 3′-O-hydroxyls are reacted with the 3′-O-protected nucleoside triphosphate under suitable conditions for elongation by the template-free polymerase, wherein the initiator or elongated fragments are elongated by incorporation of the 3′-O-protected nucleoside triphosphate to form 3′-O-protected elongated fragments, and (ii) dispensing through one or more inkjet printhead nozzles at least one droplet of a deprotection solution to deprotect the 3′-O-protected elongated fragments to form elongated fragments having free 3′-hydroxyls; and (d) repeating step (c) until the plurality of polynucleotides is synthesized,
wherein the divalent cation is a cobalt divalent cation (Co²⁺) and the divalent cation concentration is ranging from 0.1 mM to 1 mM, preferably from 0.2 to 0.5 mM, even more preferably is about 0.25 mM and the pH of the printable reagent composition is ranging from pH 5.1 to 6.6, preferably from pH 5.6 to 6.6, preferably from pH 5.6 to 6.5, more preferably from pH 5.6 to 6.4, even more preferably from pH 6.0 to 6.4, even more preferably the pH is about 6.0, or
wherein the divalent cation is a manganese divalent cation (Mn²⁺) and the divalent cation concentration is ranging from 0.5 mM to 7 mM, preferably from 1 to 6 mM, even more preferably is about 1.25 mM and the pH of the printable reagent composition is ranging from pH 6.0 to 7.0, preferably from pH 6.4 to 6.8, more preferably the pH is about 6.6, or
wherein the divalent cation is magnesium divalent cation (Mg²⁺) and the divalent cation concentration is ranging from 1 mM to 20 mM, preferably from 2 to 15 mM, preferably from 3 to 8 mM, even more preferably is about 5 mM and the pH of the printable reagent composition is ranging from pH 5.6 to 8.4, preferably from pH 7.0 to 8.2, more preferably the pH is about 8.0.

In some embodiments, the printable reagent composition is split into a first reagent composition comprising the template-free polymerase and the divalent cation; and a second reagent composition comprising the 3′-O-protected nucleoside triphosphate and the polar organic solvent. In other embodiments, the printable reagent composition is split into a first reagent composition comprising the template-free polymerase and the 3′-O-protected nucleoside triphosphate; and a second reagent composition comprising the divalent cation, and the polar organic solvent. The first reagent composition and the second reagent composition may be dispensed separately through the one or more inkjet printhead nozzles, or the first reagent composition and the second reagent composition may be mixed prior to dispensing. to form a complete printable reagent composition. In some embodiments, the mixing of said first reagent composition and said second reagent composition leads to the printable reagent composition.

In some embodiments, each printable reagent composition used in synthesis is delivered to reaction sites in separate inkjet-delivered droplets. In some embodiments, for each printable reagent composition, a single droplet or a plurality of droplets are delivered to each reaction site during each cycle of synthesis at steps (c) and (d). In some embodiments, the plurality of droplets is in the range of from 2 to 10, or in the range of from 2 to 5, or in the range of from 2 to 3. In other embodiments, the plurality of droplets may be in the range of from 2 to 150, or in the range of from 10 to 120. In some embodiments, a further step is implemented of cleaving the plurality of polynucleotides from the planar substrate. In some embodiments, a drying step may be included after step (c), or after step (d) and a washing step, to minimize spreading or coalescence of droplets when the next droplet is dispensed. In some embodiments, coupling cycles may further include a washing step after the deprotecting step. In some such embodiments, coupling cycles may further include a drying step after a washing step. As described above, a drying step prior to a successive coupling cycle prevents the spreading and possible coalescence of reaction droplets at adjacent reaction sites. In some embodiments, washing and drying can be combined by using a volatile wash solution, such as, acetonitrile, methanol, or the like, which is readily evaporated between coupling cycles.

In some embodiments, the droplet evaporation problem may be addressed by using a droplet-in-oil array as described by Sun et al (LabChip, 11: 2429-2436(2015)), in which aqueous reagents are delivered onto silicone oil droplets: (1) Mineral oil is first printed in a pattern on a silanized silicon dioxide surface with hydrophobic and oleophobic properties using a 50 μm nozzle. (2) A first round of printing of a first ink comprising aqueous reagents is performed on top of the preformed oil drops in the same pattern but with a smaller nozzle such as a 30 μm nozzle. The ejected droplets carrying reagents penetrate the oil droplets at high velocity, overcoming their surface tension and viscosity. Subsequently, the aqueous droplets sink to the bottom of the less-dense mineral oil droplets, forming stable droplet-in-oil structures. (3) During a second-pass of printing aqueous reagents, a second ink is inkjet-printed on the just formed droplet-in-oil array of the first ink. Thus, the dispensed reagent droplet also penetrates the oil drop due to its high velocity and immediately merges with the preformed droplet inside the same oil drop.

In some cases, a deprotection buffer is delivered by an inkjet generated droplet to predetermined reaction sites. In some embodiments, a droplet microarray is used comprising a substrate having a hydrophobic-hydrophilic patterned surface on which a plurality of reaction sites correspond to hydrophilic locations, each of which is capable of hosting an aqueous droplet. The aqueous droplets may be, for example, a wash solution from a previous cycle. Alternatively, as noted above, droplets of a droplet microarray may be dried after exposure to such a wash solution, so that at the beginning of a cycle, no droplets are present on the “droplet” microarray. A layer of initiators or elongated fragments having protected 3′-hydroxyls is attached to the surface of each reaction site. To a number of predetermined reaction sites, a deprotection buffer is delivered by inkjet generated droplets. The predetermined reaction sites are those in which initiators or elongated fragments are to receive a 3′-O-protected nucleoside triphosphate monomer in accordance with the predetermined polynucleotides sequences assigned to the selected reaction sites. As one of ordinary skill would appreciate, in this embodiment, monomers may have different orthogonal 3′-O-protection groups that may be removed by different deprotection buffers that permit the synthesis of multiple different polynucleotides at the same reaction site or the synthesis of DNA-RNA chimeric molecules, e.g., as described in the International patent publication of Godron et al, WO2020/141143. After an incubation time necessary to complete the deprotection reaction, one or more inks comprising a template-free polymerase, a divalent cation, and a 3′-O-protected-dATP, as described herein, are dispensed by an inkjet printhead nozzle at the reaction sites on the surface of the substrate. The droplet microarray has two types of reaction sites: those without deprotected initiators or elongated fragments, and those with deprotected initiators or elongated fragments. A coupling reaction takes place only in the droplets with deprotected initiators or elongated fragments so that a 3′-O-protected-dATP is added only at those reaction sites where the initiators or elongated fragments are deprotected. After a predetermined incubation period, the substrate is washed, and optionally, dried leaving the droplet microarray with the initiators or elongated fragments at the selected reaction sites elongated by a 3′-O-protected-dATP. The process is continued by delivering deprotection buffer to predetermined reaction sites of the droplet microarray so that initiators or elongated fragments at those sites are selectively deprotected. After an incubation time necessary to complete the deprotection reaction, one or more inks comprising a template-free polymerase, a divalent cation, and a 3′-O-protected-dCTP, as described herein, are dispensed by an inkjet printhead nozzle at the reaction sites on the surface of the substrate. A coupling reaction takes place only in the droplets with deprotected initiators or elongated fragments so that a 3′-O-protected-dCTP is added only at those reaction sites where the initiators or elongated fragments are deprotected. After a predetermined incubation period, the substrate is washed, and optionally dried, leaving droplet microarray, or dried reaction sites, with the initiators or elongated fragments at the selected reaction sites elongated by a 3′-O-protected-dCTP. A reaction cycle is completed when similar steps are performed for coupling 3′-O-protected-dGTP and 3′-O-protected-dTTP monomers to their respective sets of reaction sites. The advantage of this synthesis approach is fewer print heads, less problems due to enzyme and higher precision since enzymatic reactions will be perfectly registered with the DNA spots.

In some embodiments, inkjet-assisted synthesis may be carried out by the following steps: (a) providing a planar substrate having initiators at a plurality of distinct reaction sites, wherein each initiator has a protected 3′-hydroxyl and wherein each polynucleotide of the plurality is assigned to a different reaction site for synthesis; (b) dispensing through one or more inkjet printhead nozzles to each reaction site at which a 3′-O-protected-dATP is to be coupled in accordance with the polynucleotide assigned to such reaction site at least one droplet of a buffer solution comprising a deprotection agent; (c) dispensing to the planar substrate one or more inks comprising a template-free polymerase, a divalent cation, and a 3′-O-protected-dATP; (d) incubating the template-free polymerase, divalent cation, and 3′-O-protected-dATP so that initiators or elongated fragments at the reaction site are elongated by incorporation of a 3′-O-protected-dATP to form 3′-O-protected elongated fragments; (e) repeating steps (b), (c) and (d) for 3′-O-protected-dCTP, 3′-O-protected-dGTP and 3′-O-protected-dTTP at their respective reaction sites; and (f) repeating steps (b), (c), (d) and (e) until the plurality of polynucleotides is synthesized. As above, in some embodiments, prior to dispensing step (b) dispensing through one or more inkjet printhead nozzles, a step of drying the reaction sites may be included.

As noted above, embodiments of the method may include one or more washing steps, wherein a wash solution is flowed or sprayed on a substrate comprising an array of reaction sites. Wash solution may comprise a variety of solvents including, but not limited to, water, acetonitrile, methanol, PBS or other buffered salt solutions, or the like. In some embodiments, a wash solution may include one or more proteases, e.g., proteinase K, for the purpose of removing any polymerases that may adhere to the reaction site. In some embodiments, the method may further include a step of treating reaction sites with one or more proteases to remove or deactivate polymerases that accumulate at the reaction sites.

In some embodiments, substrates comprise reaction sites continuously enveloped by, or occupied by, a droplet. In other embodiments, reaction sites are dried between cycles of steps so that, strictly speaking, the substrate is not always, or not continuously, a droplet microarray throughout a synthesis.

In some embodiments, including those described above, the plurality of polynucleotides enzymatically synthesized (that is, the number of reaction sites) on a substrate with inkjet delivery of reagents is in the range of from 100 to 10 billion, or in the range of from 100 to 10 million, or in the range of from 100 to 100 thousand, or in the range of from 100 to 500 thousand, or in the range of from 1000 to 1 million. In some embodiments, such pluralities are synthesized on a substrate having a surface area in the range of from 1 cm² to 1 m², 1 cm² to 500 cm², or from 1 cm² to 256 cm², 1 cm² to 30 cm², or having a surface area in the range of from 1 cm² to 15 cm², or having a surface area in the range of from 1 cm² to 7 cm², or having a surface area in the range of from 7 cm² to 20 cm². In some embodiments, substrates may be prepared and undergo surface treatment after which it is cut, or diced, into smaller pieces for use. In some embodiments, the lengths of the polynucleotides synthesized in accordance with the invention are in the range of from 10 to 500 nucleotides, or in the range of from 50 to 500 nucleotides, or in the range of from 100 to 400 nucleotides, or in the range of from 100 to 500 nucleotides. In some embodiments, the per cycle coupling efficiency in the synthesis of polynucleotides in these length ranges is at least 98%, or is at least 99%, or is at least 99.5%, or is at least 99.8%, or is at least 99.9%. In some embodiments, the coupling cycle time in the synthesis of polynucleotides in these length ranges is less than 15 min per cycle, or less than 10 min per cycle, or less than 7 min per cycle, or less than 5 min per cycle.

In some embodiments, inkjet delivery of droplets may be directed to features on a substrate which have a dimension directly related to its size or area, such as a width of a square reaction site or a diameter of a round reaction site. Thus, in some embodiments, reaction sites have a width or diameter in the range from about 10 μm to about 1.0 cm. In some embodiments droplets can be deposited to reaction sites whose widths, or diameters, are in the range of from about 1.0 μm to about 1.0 mm, usually about 5.0 μm to 500 μm, more usually about 10 μm to 200 μm, and still more usually from about 20 μm to about 100 μm.

In some embodiments, the volume of reagent ink delivered to a reaction site is in the range of 0.1 to 1000 pL, or in the range of from 0.5 to 500 pL, or in the range of from 1.0 to 250 pL, or in the range of from 1.0 to 100 pL, or in the range of from 2 to 50 pL, or in the range of from 1 to 30 pL. In some embodiments, reagent ink is delivered to each reaction site in a predetermined number of droplets, or “pulses,” generated by a print head wherein, for example, each pulse has about a 2.4 picoliter volume.

### Apparatus for Inkjet Synthesis

Delivering fluids by inkjets is a mature technology that has been available for several decades so that extensive literature is available describing it and providing guidance for adapting it to novel applications, such as for inkjet-assisted synthesis of polynucleotides, as described herein. Exemplary references providing guidance for constructing inkjet delivery systems: Lausted et al, Genome Biology, 5: R58 (2004); Le, Recent Progress in Ink Jet Technologies II, chapter 1, pgs. 1-14 (1999); Derby (2010, cited above); Zapka, editor, “Handbook of Industrial Inkjet Printing,” (Wiley-VCH, Weinheim, Germany); U.S. Pat. Nos. 5,474,796; 10,384,189; 10,669,304; 6306; 6,323,043; 5,847,105; and the like. As noted by Le (1999) inkjet printhead nozzles may be classified as “continuous” and “drop-on-demand” (DOD). In some embodiments, DOD inkjet printhead nozzles are employed with apparatus of the invention, and in particular, of the various DOD inkjets, piezoelectric inkjet printhead nozzles are of interest. For example, droplet formation in DOD inkjets is described in Dong et al, Physics of Fluids, 18: 072102 (2006). Such varieties of inkjet printhead nozzles are available banks or assemblies of large numbers of inkjets (e.g. from 10's to 100's) that may be individually programmed for actuation and delivery of droplets. Such inkjets and inkjet assemblies (referred to herein as “inkjet heads”) are commercially available from many manufacturers including Epson, Xaar, Fujifilm, and the like. As used herein, “inkjet printhead nozzle” means a device capable of generating and ejecting droplets of a fluid. In some embodiments, an inkjet printhead nozzle is a device capable of generating and ejecting droplets of a fluid at a predetermined rate and of a predetermined uniform size. In some embodiments, an inkjet printhead nozzle is capable of ejecting droplets each having approximately the same size in the range of from 0.1 pL to 5.0 nL, or the same size in the range of from 0.5 pL to 1.0 nL. In some embodiments, an inkjet printhead nozzle is capable of ejecting droplets at a rate in the range of from 1 to 100 kilohertz.

In some embodiments, components of an inkjet apparatus may be arranged according to whether they may be moved relative to one another or whether they are fixed. Computer and software provide overall control of the system components, either directly or indirectly via controllers. For example, software may provide for single pass reagent deposition in which print head is stationary and synthesis support holder moves to deliver reagents to reaction sites. Alternatively, different software may provide for one or more moving print heads and/or moving synthesis support holder via a variety of components, such as, a print controller, print head driver and motion controller. Typically, computer and software control capping station, flush station, wiper, inspection system and washing and drying functions are included. A capping station keeps the print head moist and stops drying of ink. A flush station primes and flushes the print head, which helps remove trapped air and debris as well as dried ink. A wiper is used to remove excess ink and prevent cross-contamination. It may be part of the flush station. An inspection system records the presence, absence or size of spots of deposited reagents or incorrectly placed spots of reagents. The inspection system may comprise a camera that takes images of the synthesis support, and image analysis software extracts and processes information from the images. Such information may be used in real-time to optimize synthesis or to implement corrective measures. Washing and drying functions are carried out by a fluid delivery system separate from that used for droplet delivery. Washing may include deprotection steps, wherein a deprotection reagent is flowed across a synthesis substrate, optionally followed by a drying step. Drying may be accomplished by blowing air or an inert gas, such as argon, over the synthesis support, or by using a volatile solvent, such as methanol, in the washing step.

In some embodiments, cameras or microscopes may be used to capture images of the spots (i.e., reaction sites) and identify missing spots, determine spot size and spot placement. Lighting for image capture may be from above, from the side, from below or integrated into a substrate holder, whichever gives the best contrast in the absence or presence of dye in the inks. Where a dye is used, it is selected so that it does not interfere with the enzymatic reaction, does not react with the protecting group of the nucleotide, and is compatible with the enzyme and deprotection buffers. In some embodiments, each composition comprising a monomer has a different distinguishable dye, covering a different part of the visible spectrum. In some embodiments, imaging of an array of reaction site is carried out during incubation (30 s-10 min) of the elongation reactions and using high enough magnification to see individual spots but not so high that an inordinate length of time would be needed to scan the array. The number of images taken in an imaging step may be 20 to 100 for a standard microscope slide. Images may be captured seamlessly in a video stream by scanning the substrate or captured in a move-stop process. The images captured may be stitched using algorithms and aided by the presence of fiducial markings on the slide. Fiducial markings also help determine whether the slide has moved in the slide holder and help determine spot positions. In some embodiments, real time image analysis allowing the identification of missing spots or poor spot placement could be accompanied by the automatic generation of a new image and an additional print or prints.

In some embodiments, a plurality of DOD inkjets are housed in print head which is capable of x-y and z movement relative to droplet microarray. In some embodiments both print head and the droplet microarray are capable of x-y movement. In some embodiments, the print head is held in a fixed position and the droplet array undergoes x-y movement. The print head may further comprise containers containing printable reagent compositions or inks comprising a template-free polymerase, respective 3′-O-protected-dNTPs, salts and cofactors necessary or useful for polymerase activity, as well as viscosity and surface tension modifiers, humectants, and the like, as needed to meet the requirements for desired droplet formation and/or to reduce evaporation loss. The print head may also include temperature regulation to maintain the inks at a temperature optimized for delivery and activity. In some embodiments, some reagents are flowed or delivered to the droplet microarray in bulk such as the deprotection solution and wash solutions. The droplet microarray, which is formed on the substrate, sits or is mounted in a flow chamber, which comprises an inlet and outlet. A flow chamber defines the flow path of reagents (not delivered by the print head) over the droplet microarray. Such reagents may flow continuously over the droplet microarray, or reagents may be delivered to the flow chamber where they remain for a predetermined incubation time, and then are removed or recycled. Such reagents may be moved by conventional pumps or by pressure heads over reagent reservoirs. The flow chamber may also include temperature control elements and humidity control elements to maintain or optimize coupling reaction activity. After exiting, reagents are discarded into a waste container or recycled. Timing of inkjet discharges, positioning of print head, actuation of valves is controlled by fluidics/inkjet controller, which may include imaging software that performs analysis of array images obtained by a camera and that causes alterations of reagent deposition, for example, when coalescing reaction sites are detected. In some embodiments, the print head may be driven by electronics available from Meteor (Meteor Inkjet Ltd, (Cambridge, UK). For example, a Print Controller Card (PCC) can be used that synchronizes to the encoder signal from a Thorlabs motion controller. A Head Driver Card (HDC) provides power and a waveform to the printhead. The drive electronics are controlled by Meteor's digital printing front end, which includes MetDrop and MetWave software for optimization of spotting parameters, with printing initiated by the Thorlabs Kinesis software. Overall instrument control can be performed by instrument software, such as LabView.

Typically, the distance between the inkjet nozzles and the substrate surface may be in the range of from about 10 μm to 10 mm, or in the range of from about 100 μm to 2 mm, or in the range of from about 200 μm to 1 mm, or in the range of from 500 μm to 3 mm. Droplet velocities may be in the range 1-10 meters/sec. Print head movement may be in the range of from 1-30 cm/sec, or 5-30 cm/sec, or 20-30 cm/sec. As described more fully below, print heads may have different droplet delivery modes, for example, single-pass mode, multiple pass mode, and move-stop mode.

As mentioned above, in some embodiments, nozzle diameters for use in printing of reagent compositions may be in the range of from 10 μm to 100 μm. In other embodiments, the inkjet nozzle size may be in the range of from 20-30 μm for generating droplet sizes in the range of from 10-20 pL. In some embodiments, the nozzle diameter, synthesis reagent density, surface tension and viscosity are selected to dispense droplets to reaction sites having a volume in the range of from 2 pL to 5 nL, or in the range of from 2 pL to 1 nL, or in the range from 2 pL to 500 pL, or in the range from 2 pL to 100 pL. In some embodiments, inkjet printhead nozzles are DOD inkjet printhead nozzles and have a droplet generation rate in the range of from 1 to 100 kHz.

In some embodiments, inkjet-based synthesizers include droplet detection components to monitor and record any anomalies in droplet formation and delivery by the inkjet nozzles. In some embodiments, such droplet monitoring may comprise a laser diode mounted orthogonally to the direction of print-head motion such that the droplet stream of each bank of nozzles intersects the beam, causing the light to scatter if a droplet is present. Before each round of printing, nozzles may be fired in series through the beam and the forward scattering of each droplet is detected by a photodiode. Nozzles failing to fire may be taken off-line during synthesis. The inkjet apparatus may also be equipped with commercially available droplet monitors, such as, a Meteor dropwatcher, available from Meteor Inkjet Ltd, (Cambridge, UK) as well as a camera to image the solid support and array of reaction sites. The latter permits the array of reaction sites to be monitored to detect accuracy in droplet deposition, size and geometry of reaction sites, coalescence of reaction sites, and the like. In some embodiments, software may be provided to provide a full image of an array on a slide or solid support by patching together tiles comprising smaller images, e.g. S. Preibisch, S. Saalfeld, P. Tomancak, Bioinformatics, 2009, 25(11), 1463-1465.

In some embodiments, it may be desirable to prevent evaporation of the synthesis reagents and reaction mixtures following deposition. Evaporation may be prevented in a number of different ways. In some embodiments, synthesis cycles may be carried out in a high humidity environment, such as a relative humidity in the range of from 75-85%. Alternatively or in addition to, one may employ reagents with an evaporation retarding agent or humectant, e.g. glycerol, glycerol acetate, polyethylene glycol, carboxymethyl cellulose, hydroxyethyl cellulose, and the like.

In some embodiments, recirculating ink print heads are employed because problems of drying and/or clogging of nozzles by enzymes is reduced. Recirculating ink print heads are commercially available, for example, from Fujifilm and are described in U.S. Pat. Nos. 8,820,899; 8,534,807; 8,752,946; 9,144,993; 9,511,598; 9,457,579, which are incorporated herein by reference.

### Synthesis Substrates

In some embodiments, substrates for synthesis comprise surfaces that have been patterned with hydrophobic and hydrophilic regions wherein discrete hydrophilic reaction sites are formed. These allow the formation of droplets on hydrophilic reaction sites, for example, after flowing aqueous reagents or reactants of the entire surface. That is, in some embodiments, substrates for synthesis comprise so-called “droplet microarrays,” e.g., as disclosed in the following exemplary references, which are incorporated by reference: Brennan, U.S. Pat. No. 5,474,796; Chrisey et al, Nucleic Acids Research, 24(15): 3040-3047 (1996); Fixe et al, Materials Research Society Symposium Proceedings. Volume 723.

Molecularly Imprinted Materials—Sensors and Other Devices. Symposia (San Francisco, California on Apr. 2-5, 2002); Goldfarb, U.S. patent publication 2008/0166667; Gopinath et al, ACS Nano, 8(12): 12030-12040 (2014); Hong et al, Microfluid. Nanofluid., 10: 991-997 (2011); Kumar et al, Nucleic Acids Research, 28(14): e71 (2000); Peck et al, U.S. patent Ser. No. 10/384,189; Indermuhle et al, U.S. patent Ser. No. 10/669,304; Wu et al, Thin Solid Films, 515: 4203-4208 (2007); Zhang et al, J. Phys. Chem., 111: 14521-14529 (2007); and like references. As used herein, the term “droplet microarray” refers to a planar substrate whose surface has been treated to create a plurality of discrete hydrophilic regions, which may serve as reaction sites either directly or with further treatment, e.g. attaching initiators. In some embodiments, each of the plurality of discrete hydrophilic regions are surrounded by hydrophobic regions. The discrete hydrophilic regions may have a variety of shapes, but are usually circular or rectangular or square for manufacturing convenience. In some embodiments, reaction sites have areas and capacities to hold an aqueous reaction mixture as described above. Although synthesis substrates of some embodiments may comprise droplet microarrays, in a synthesis process such arrays may undergo a drying step which removes liquid from reaction sites. That is, in some embodiments, a synthesis substrate comprising a droplet microarray may be devoid of droplets from time to time, for example, after an elongation cycle ending in a drying step. The hydrophilic-hydrophobic configurations permit the formation of droplets on the surface of a droplet microarray either after inkjet delivery of a synthesis reagent to the hydrophilic regions or by flowing a “bulk” aqueous solution, such as a synthesis reagent or wash solution, over the substrate. As disclosed in the above references, the droplets retained by the hydrophilic regions may serve as reaction chambers or vessels. The planar substrate has a surface with hydrophobic region and discrete hydrophilic regions, which may serve as reaction sites. When the planar substrate is flooded with an aqueous solution both hydrophobic regions and hydrophilic regions are immersed. When the aqueous solution drains off, some of the aqueous solution is retained by hydrophilic regions to form droplets of the droplet microarray. Individual droplets may be referred to as a “microarray droplet” to distinguish them from droplets formed by an inkjet printhead nozzle prior to its delivery to a reaction site.

Preparation of substrates with discrete reaction sites can be accomplished by known methods. For example, such methods can involve the creation of hydrophilic reaction sites by first applying a protectant, or resist, over selected areas over the surface of a substrate, such as a silicon oxide, or like material. The unprotected areas are then coated with a hydrophobic agent to yield an unreactive surface. For example, a hydrophobic coating can be created by chemical vapor deposition of (tridecafluorotetrahydrooctyl)-triethoxysilane onto the exposed oxide surrounding the protected circles. Finally, the protectant, or resist, is removed exposing the well regions of the array for further modification and nucleoside synthesis using the high surface tension solvents described herein and procedures known in the art such as those described by Maskos & Southern, Nucl. Acids Res. 20:1679-1684 (1992). Alternatively, the entire surface of a glass plate substrate can be coated with hydrophobic material, such as 3-(1,1-dihydroperfluoroctyloxy)propyltriethoxysilane, which is ablated at desired loci to expose the underlying silicon dioxide glass. The substrate is then coated with glycidyloxypropyl trimethoxysilane, which reacts only with the glass, and which is subsequently “treated” with hexaethylene glycol and sulfuric acid to form an hydroxyl group-bearing linker upon which chemical species can be synthesized (Brennan, U.S. Pat. No. 5,474,796). Arrays produced in such a manner can localize small volumes of solvent within the reaction site by virtue of surface tension effects (Lopez et al., Science 260:647-649 (1993)).

In some embodiments, reaction sites may be formed on a substrate following the photolithographic methods of Brennan, U.S. Pat. No. 5,474,796; Peck et al, U.S. patent Ser. No. 10/384,189; Indermuhle et al, U.S. patent Ser. No. 10/669,304; Fixe et al (cited above); or like references cited above. In accordance with these methods, a set of hydrophilic molecules comprising an aminosilane is attached to the surface of a substrate to form reaction sites. Such hydrophilic molecules may comprise N-(3-triethoxysilylpropyl)-4-hydroxybutyramide (HAPS), 11-acetoxyundecyltriethoxysilane, n-decyltriethoxysilane, (3-aminopropyl)trimethoxysilane, (3-aminopropyl)triethoxysilane, 3-glycidoxypropyltrimethoxysilane (GOPS), or 3-iodo-propyltrimethoxysilane. A set of hydrophobic molecules comprising a fluorosilane is attached to the surface of the substrate in regions outside of the reaction sites. Such hydrophobic molecules may comprise perfluorooctyltrichlorosilane octylchlorosilane, octadecyltrichlorosilane, (tridecafluoro-1,1,2,2-tetrahydrooctyl)trichlorosilane, or tridecafluoro-1,1,2,2-tetrahydrooctyl)trimethoxysilane. After such attachment, a substrate is prepared for polynucleotide synthesis by coupling initiators to the aminosilanes at the reaction sites. Such coupling may be accomplished using any number of available homo- or heterobifunctional linkers to form covalent bonds between amino groups on the substrate and 5′-thiol groups or 5′-amino groups on the initiators. Such linkers are, for example, available from Sigma-Aldrich (St. Louis, MO) and are described in treatises such as, Hermanson, Bioconjugate Techniques, 3^{rd} Edition (Academic Press, 2013). Synthesis of oligonucleotides having 5′-thiol or 5′-amino groups is well-know and is described in Kupihar et al, Nucleosides Nucleotides & Nucleic Acids, 22(5-8): 1297-1299 (2003); Fung et al, U.S. Pat. No. 4,757,141; and like references.

In some embodiments, an array of reaction sites may be formed using click chemistry by depositing under coupling conditions droplets of 5′-DBCO (dibenzocyclooctyl) labeled initiators (e.g. Glen Research) on a planar substrate comprising an azide layer (e.g. PolyAn 2D azide glass slide). In some embodiments, such reactions may be carried out as a copper-free click reaction which is less damaging to the DNA, e.g. Dommerholt et al, Top. Curr. Chem. (Z) 374: 16 (2016).

A wide variety of substrates may be employed for creating arrays of reaction sites for enzymatic synthesis of polynucleotides. Substrates may be a rigid material including, without limitation, glass; fused silica; silicon such as silicon dioxide or silicon nitride; metals such as gold or platinum; plastics such as polytetrafluoroethylene, polypropylene, polystyrene, polycarbonate, and any combination thereof. A rigid surface can be fabricated from a material selected from the group consisting of silicon, polystyrene, agarose, dextran, cellulosic polymers, polyacrylamides, polydimethylsiloxane (PDMS), and glass. Substrates may also comprise flexible materials, which is capable of being bent, folded or similarly manipulated without breakage. Exemplary flexible materials include, without limitation, nylon (unmodified nylon, modified nylon, clear nylon), nitrocellulose, polypropylene, polycarbonate, polyethylene, polyurethane, polystyrene, acetal, acrylic, acrylonitrile, butadiene styrene (ABS), polyester films such as polyethylene terephthalate, polymethyl methacrylate or other acrylics, polyvinyl chloride or other vinyl resin, transparent PVC foil, transparent foil for printers, Poly(methyl methacrylate) (PMMA), methacrylate copolymers, styrenic polymers, high refractive index polymers, fluorine-containing polymers, polyethersulfone, polyimides containing an alicyclic structure, rubber, fabric, metal foils, and any combination thereof.

In some embodiments, patterned surfaces of superhydrophobic and superhydrophilic regions may be formed on a substrate. Guidance for forming droplet microarrays with such patterned surfaces are described in the following references, which are incorporated by reference: Feng et al, Adv. Mater. Interfaces, 1400269 (2014); Zhan et al, Trends Anal. Chem., 108: 183-194 (2018); Neto et al, Adv. Functional Mater., 201400503 (2014);

Achieving accurate alignment of droplet delivery to reaction sites of a prefabricated droplet microarray is an important aspect of inkjet-assisted synthesis of polynucleotides. In some embodiments, such alignment tasks may be minimized or avoided by creating immediately prior to synthesis an array of reaction sites by depositing droplets of synthesis reagents onto a layer of initiator oligonucleotides on a substrate in order to define the locations of reaction sites. Following this initial deposit of droplets, the initiator layer outside of the droplet-defined sites are treated to render them inert to subsequent extension or to render them inert to extension as well as hydrophobic. After such an initial surface treatment to create reaction sites, further or subsequent inkjet delivery of droplets to the same reaction sites will be accurate because the same inkjet printhead nozzles that were used to define the locations of the reaction sites will be used to deliver subsequent droplets during synthesis of the polynucleotides. In some embodiments, the synthesis reagents delivered to the initiator layer comprise a mixture of a template-free polymerase and a 3′-O-protected-dNTP. These reagents extend the initiators to define reaction sites or regions on the oligonucleotide layer which is populated by extended fragments having 3′-O-protected ends. The areas outside of these regions are then treated to render them inert to extensions. In some embodiments, after the initial coupling step defining reaction sites, the entire substrate is exposed to a template-free polymerase and a terminator, such as a dideoxynucleoside triphosphate (ddNTP), or like reagent. In some embodiments, such ddNTP could be, for example, a ddNTP conjugated to a hydrophobic moiety, thereby rendering the coating outside of the reaction sites hydrophobic. Such a hydrophobic moiety may be, for example, a dye or quencher molecule, such as, a Black Hole Quencher® molecule. A variety of terminators may be employed for this purpose. In particular, terminators include nucleoside triphosphates that lack a 3′-hydroxyl substituent and include 2′,3′-dideoxyribose, 2′,3′-didehydroribose, and 2′,3′-dideoxy-3′-haloribose, e.g. 3′-deoxy-3′-fluoro-ribose or 2′,3′-dideoxy-3′-fluororibose nucleosides. Alternatively, a ribofuranose analog can be used in terminators, such as 2′,3′-dideoxy-β-D-ribofuranosyl, 3-D-arabinofuranosyl, 3′-deoxy-β-D-arabinofuranosyl, or the like. Further terminators are disclosed in the following references: Chidgeavadze et al., Nucleic Acids Res., 12: 1671-1686 (1984); Chidgeavadze et al., FEBS Lett., 183: 275-278 (1985); Izuta et al, Nucleosides & Nucleotides, 15: 683-692 (1996); and Krayevsky et al, Nucleosides & Nucleotides, 7: 613-617 (1988). Nucleotide terminators also include reversible nucleotide terminators, e.g. Metzker et al. Nucleic Acids Res., 22(20):4259 (1994).

Thus, in such embodiments, a starting material for a synthesis operation is a surface coated with a layer of initiator oligonucleotides. The planar substrate (e.g., a glass slide) has a layer of initiator oligonucleotides that have free 3′-hydroxyl groups and that are attached by their 5′-ends to the substrate. In some embodiments, initiator densities may be, for example, in the range of from 10¹¹ to 10¹³ strands/cm². Inkjet printhead nozzles in inkjet head are used to deposit droplets in a regular and repeatable pattern on layer that define reaction sites. For example, the 3′-hydroxyls of such initiators may be unprotected and the droplets may contain a template-free polymerase and an initial 3′-O-protected nucleoside triphosphate, thereby producing 3′-O-protected elongated fragments in each reaction site. After such deposition, the layer of initiators is immersed and incubated in a buffer comprising a template-free polymerase and a terminator, e.g., as described above, to produce droplet microarray having a surface outside of the reaction sites inert to extension or inert to extension and hydrophobic depending on the terminator selected.

An aspect of the invention is a method for preparing an array of reaction sites for template-free enzymatic synthesis of a plurality of polynucleotides. In some embodiments, such method of array preparation may be carried out by the steps of (a) providing a surface with initiators attached, (b) delivering with one or more inkjet printhead nozzles droplets to a plurality of distinct locations on the surface to form a plurality reaction sites, the droplets containing a synthesis reagent that reacts with initiators in the reaction sites to remove 3′-O-protecting groups or to elongate such initiators by addition of a 3′-O-protected nucleoside triphosphate, and (c) capping initiators on the surface outside of the reaction sites. In some embodiments, initiators on the surface of step (a) have free 3′-hydroxyls and the synthesis reagent delivered in step (b) comprises a template-free polymerase and a 3′-O-protected nucleoside triphosphate, so that the template-free polymerase catalyzes the addition of the 3′-O-protected nucleoside triphosphate to produce 3′-O-protected elongated fragments within the reaction sites. Thus, initiators outside of the reaction sites may be capped by immersion of the surface in a capping reagent (such as a mixture containing a dideoxynucleoside triphosphate and template-free polymerase). In some embodiments, initiators on the surface may have 3′-O-protection groups and the synthesis reagent delivered by droplets may contain a deprotection agent that removes the 3′-O-protection groups from initiators to form reaction sites. In the newly formed reaction sites, a reagent is delivered which contains 3′-O-protected nucleoside triphosphates and a template-free polymerase, wherein the protection group of the delivered nucleoside triphosphate is orthogonal to that of the initiators of the surface. Exemplary orthogonal 3′-O-protection groups are described below. For example, such orthogonal protection groups may be azidomethyl and amino.

In another embodiment, a beginning layer of initiator oligonucleotides all have 3′-O-amino-protected or 3′-O-azidomethyl-protected ends. A deprotection buffer is inkjet printed on the substrate to define reaction sites as discrete regions of initiators having free 3′-hydroxyls. After such selective deprotection, the surface is treated with an aqueous solution of an aldehyde or ketone to form a stable non-extendable hydrophilic or hydrophobic 3′-oxime. The aldehyde or ketone may be water soluble, e.g., an acetone, or slightly water soluble and hydrophobic (e.g., a pentanal, aldehyde-PEG-DBCO, or the like), or very hydrophobic and water insoluble (e.g., a heptanal).

In another embodiment, a buffer comprising a template-free polymerase/3′-O-protected-dNTP mixture is printed on the initiator oligonucleotide layer with free 3′-hydroxyls as described above to define reaction sites having extended initiators with 3′-O-protected ends. The surface outside these defined sites is then treated with a template-free polymerase and an azide or alkyne derivatized ddNTP to block further 3′ extensions. A hydrophobic molecule with a complementary click chemistry group (e.g. DBCO, benzyl-azide) may then be reacted with the ddNTP terminator to render the surface outside of the reaction sites hydrophobic. Exemplary click chemistry pairs are described in Feng et al, Adv. Mater. Interfaces, 1400269 (2014).

In still another embodiment, to a substrate surface without a layer of initiator oligonucleotides, a buffer comprising initiator oligonucleotides having 5′ linker groups is inkjet printed on the surface derivatized with a complementary reactive group (e.g. epoxy, azide/alkyne) so that the initiators are attached to the surface by their 5′-ends. To these attached initiators, cycles of coupling reactions can take place in accordance with the invention. Also, unreacted complementary reactive groups may be quenched by reacting them with an inert group (e.g., ethanolamine for epoxy) and the inert group may be selected to have a hydrophobic character.

In some embodiments, substrates for synthesis may include surface-bound cDNAs copied from messenger RNA extracted from a fixed or non-fixed tissue slice. Procedures for placing tissue slices on a planar array of oligonucleotides, identifying and imaging tissue features (such as cell boundaries), permeabilizing cells of tissues, implementing reverse transcriptase reactions to produce a cDNA library attached to a planar array are disclosed in Stahl et al, Science, 353: 78-82 (2016); and Frisen et al, U.S. Pat. Nos. 9,593,365 and 10,030,261; and like references, which are incorporated herein by reference. In some embodiments, a planar array is provided with a uniform coating of oligonucleotides, with a controlled density, or predetermined density, attached by their 5′ ends, wherein the oligonucleotides comprise a segment comprising a primer binding site for later amplification and manipulation of a cDNA, an optional segment comprising a molecular tag (sometimes referred to as a “unique molecular identifier” or UMI), which facilitates quantification of cDNA molecules even after amplification, and a segment comprising a polyT sequence, which permits capture of mRNA released from cells. The UMI may comprise a random nucleotide segment. Oligonucleotides may be made in bulk using conventional techniques and applied to the surface of the planar array in a single step. Different kinds of oligonucleotides, for example, oligonucleotides with different position tags are not required. A cleavable linker or cleavable nucleotide may be included for releasing cDNAs for analysis, such as, by sequencing. Onto the array is disposed a slice or thin layer (e.g. 100-1000 μm thick) of tissue, which is then treated (i) to identify features, such as cells or sub-tissues, of interest and to record and/or correlate such information to locations on the planar array, and (ii) to permeabilize cells in the tissue so that mRNA is released and allowed to diffuse to and be captured by oligonucleotides.

The image information is used to define regions on the array within which common position tags are synthesized on cDNAs. Treatments may include staining with tissue-specific or biomolecule-specific compounds or dyes. The position tags allow cDNAs to be harvested and sequenced in bulk, yet be related to specific regions by their position tags. After the above steps (i) and (ii), reagents for a reverse transcriptase reaction are applied in order to synthesize cDNAs using captured mRNAs as templates to produce a spatial cDNA library array. Tissue slice is then removed leaving array with a pattern of different cDNAs attached to its surface. The different cDNAs at the different positions may be identified and quantified by attaching position tags to samples of cDNAs from a plurality of locations by inkjet delivery of synthesis reagents for the tags, by the superposition of synthesis locations on the cDNA pattern. In some embodiments, such plurality may be at least 100 positions, or at least 1000 positions, or at least 10,000 positions; in other embodiments, such plurality may be in the range of from 10 to 50,000 positions; or from 10 to 10,000 positions; or from 10 to 1000 positions. Guidance for design and control of inkjet delivery systems is well known by those with skill in the art and may be found in U.S. patent publication US2003/0170698 and U.S. Pat. Nos. 6,306,599; 6,323,043; 7,276,336; 7,534,561; and like references. Position tags are selected (e.g., are long enough) to uniquely identify each location or region of interest. Additional segments may be added to facilitate manipulation and sequencing of cDNAs.

In some embodiments, this application may be carried out with the following steps: (a) providing an array comprising a uniform coating of capture probes each comprising a capture segment; (b) contacting a tissue sample with the array and allowing the nucleic acid of the tissue sample to interact with the capture domain of the capture probe so that the nucleic acid is captured; (c) treating the tissue sample to identify different regions of the tissue sample; (d) generating a nucleic acid molecule from the nucleic acid that interacts with the capture domain; (e) enzymatically synthesizing position tags onto the nucleic acid molecules; (f) determining the region that is associated with the nucleic acid that interacts with the capture domain; and (e) correlating the determined regions to the cDNAs. In some embodiments, the nucleic acid molecules from the tissue sample is RNA. In other embodiments, the nucleic acid molecules from the tissue sample may be genomic DNA. In other embodiments, the nucleic acid molecules from the tissue sample may be mRNA. In some embodiments, the step of enzymatically synthesizing position tags onto the nucleic acid molecules is carried out by inkjet delivery of synthesis reagents to the locations of the position tags in accordance with methods of the present invention.

### Overwriting for High Density Barcode Synthesis

In typical inkjet synthesis applications, an array of distinct non-overlapping reaction sites is defined by repeated deposition of reagents. Usually, such reaction sites are roughly circular regions having diameters in the range of from about 20-50 μm. Thus, for some applications, the spatial resolution achievable by inkjet printed barcodes is very limited, especially if intracellular resolution is desired. In some embodiments, a higher density of unique barcodes may be synthesized on a surface by overwriting one array of oligonucleotides with another overlapping array of oligonucleotides. In other words, a first array of reaction sites may be defined on a surface on which a first set of oligonucleotides is synthesized, after which a second array of reaction sites is defined the same surface on which a second set of oligonucleotides is synthesized, such that the second array overlaps the first array so that the surface is partition into a larger number of smaller-sized regions in which there are unique barcodes. In some embodiments, for example, if the oligonucleotides of the first set are each m nucleotides in length with different predetermined, or known, sequences and the oligonucleotides of the second set are each n nucleotides in length, also with different predetermined, or known sequences, then there will be regions of the surface containing oligonucleotides that are m nucleotides in length, n nucleotides in length and n+m nucleotides in length, each with a known unique sequence in a known region. In other embodiments, oligonucleotides of selected reaction sites within the arrays may be capped to prevent further extensions.

In some embodiments, the substrate may be physically positioned by angle stop so that the print head of an inkjet printing instrument can be programmed to have a well-defined starting location for producing reaction sites of an array. For example, a print head with multiple nozzles (e.g., two, three, four, or five, or more nozzles) can be used with a predetermined “Y” axis spacing dependent on the angle of the print head with respect to the substrate (for example, angle “a” with respect of the “X” axis). The print head is perpendicular to the “X” axis, so that the rows of reaction sites have the maximum separation in the “Y” direction. The separation of reaction sites in the “X” direction is determined by (i) the predetermined frequency at which the nozzles generate and emit droplets and (ii) the speed at which the print head traverses the substrate. The separation of reaction sites in the “Y” direction may be determined by rotating the print head relative to the “X” and “Y” axes. That is, a predetermined angle “a” less than 90 degrees may be selected to determine inter-row distance in the “Y” direction. This produces an array of reaction sites that is roughly parallelogram shaped and having origin, or reference, reaction site. Oligonucleotides, i₁₁, i₁₂, . . . i₁ₛ, i₂₁, i₂₂, . . . i₂ₛ, . . . iₖ₁, iₖ₂, . . . iₖₛ, may be synthesized in the reaction sites to produce an s by k array of barcodes, or barcode segments, which may or may not have distinct sequences from other barcode segments. Each of the reaction sites in the array has a predetermined position relative to the reference, or origin, reaction site.

In some cases, overwriting may increase the density of spatial barcoding. The substrate may comprise an array of oligonucleotides i₁₁, i₁₂, . . . having an origin, or reference position. On top of the array, an array of oligonucleotides j₁₁, j₁₂, . . . having an origin, or reference position, is synthesized. In some embodiments, the arrays have identical X and Y spacing and angle “a”. The only geometrical or spatial difference between the arrays is that the origin of array is translated distance in the X direction relative to array. This creates a host of new regions on the substrate, each with a barcode, which by judicious selection of sequences of oligonucleotides i₁₁, i₁₂, . . . , and oligonucleotides j₁₁, j₁₂, . . . may be unique. In this particular example, each row of four subregions of an area is transformed into a row of 15 subregions in the same area: end region with oligonucleotide r₁₁=j₁₁, almond-shaped region with oligonucleotide r₁₂=i₁₁+j₁₁, hourglass region with oligonucleotide r₁₃=j₁₁, almond-shaped region with oligonucleotide r₁₄=i₁₂+j₁₁, hourglass region with oligonucleotide r₁₅=j₁₂, and so on. (The terminology “i₁₂+j₁₁”, and like terms, mean that a composite oligonucleotide is synthesized. For example, if i₁₂ is 5′-AATCCG-3′ and j₁₁ is 5′-TTGGA-3′, then the oligonucleotide iᵢ₂+j₁₁ is “5′-AATCCGTTGGA-3′—SEQ ID NO:1). In some embodiments, the lengths of oligonucleotide in such overlapping arrays are selected so that each region has a unique barcode. Thus, the lengths depend in part on how many reaction sites are present in the arrays. In some embodiments, the lengths of each oligonucleotide is in the range of between 2 and 12 nucleotides, or between 3 and 8 nucleotides. The lengths of oligonucleotides in successive arrays may be the same or different.

The translation of the positions of a subsequent array relative to a previous array may be carried out using two XY-stages, one to move the print head to generate an array of oligonucleotides, and another to provide the offset or new position of the reference reaction site relative to the reference reaction site of a previously synthesized array. The two XY stages may be used in tandem, e.g. one mounted on top of the other, or one may be used to move the print head, which the other moves the mounting stage holding the substrate. In other embodiments, a single XY stage that can be programmed to generate the desired offsets may be used. For example, Offsets can also be introduced by using high dpi print heads and choosing whether to switch on certain nozzles in the y direction that were not previously being used.

In some embodiments, overlapping arrays need not be produced only by simple translations in the X direction or the Y direction alone. Overlapping arrays may be produced by translations in both the X direction and the Y direction. A complicated pattern of regions may be produced in which different barcodes are synthesized, which may be distinct and unique from every other barcode by judicious selection of the lengths and sequences of the i and j subunits.

In some embodiments a plurality of overwritings based on a plurality of translations of an array of reaction sites may be carried out. For example, a first array is shown as a rectilinear array of four reaction sites each containing different oligonucleotides i₁₁, i₁₂, and so on. The origin of the array is shifted a predetermined distance in the X direction after which a second set of oligonucleotides j₁₁, j₁₂, . . . (shaded circles) is synthesized, to produce additional regions. After such second synthesis, the origin of the array is again shifted a predetermined distance in the negative Y direction after which a third set of oligonucleotides k₁₁, k₁₂, . . . is synthesized to produce a pattern of regions. The origin of the array may be shifted a predetermined distance in the negative X direction a third time, after which a fourth set of oligonucleotides l₁₁, l₁₂, . . . is synthesized. This produces a pattern of regions each capable of having a unique barcode, which may comprise a single i, j, k, or l segment, or may comprise a composite of up to all four such components.

In some embodiments, a method of enzymatically synthesizing a plurality of oligonucleotide barcodes each having a predetermined sequence at distinct predetermined regions of substrate is provided, the method comprising the steps of: (a) providing a substrate with a surface comprising a coating of initiators, wherein each initiator has a free 3′-hydroxyl; (b) determining a position of a reference reaction site of an array of a plurality of reaction sites, the position of each reference reaction site after the first reference site position is selected so that at least one reaction site of the array overlaps a reaction site of the previous array; (c) synthesizing an oligonucleotide in each reaction site of the array by (i) dispensing through one or more inkjet printhead nozzles at least one droplet of at least one ink comprising synthesis reagents to each reaction site of the plurality to perform a reaction cycle comprising the steps of (A) reacting under elongation conditions the initiator or elongated fragments having free 3′-O-hydroxyls with a 3′-O-protected nucleoside triphosphate and a template-free DNA polymerase so that the initiator or elongated fragments are elongated by incorporation of a 3′-O-protected nucleoside triphosphate to form 3′-O-protected elongated fragments, and (B) deprotecting the elongated fragments to form elongated fragments having free 3′-hydroxyls, wherein the synthesis reagent comprises a template-free polymerase, a 3′-O-protected nucleoside triphosphate, a mixture of a template-free polymerase and a 3′-O-protected nucleoside triphosphate, or a deprotection solution; and (ii) repeating a predetermined number of times step (i) until the plurality of oligonucleotides is synthesized; (c) repeating steps (b) and (c) until the plurality of oligonucleotide barcodes are synthesized each having a predetermined sequence at predetermined regions of the substrate comprising overlapping and non-overlapping reaction sites. In some embodiments, each array of step (b) is the same except for the location of its reference reaction site. In some embodiments, each array of step (b) has the same plurality of reaction sites and the same pattern and inter-reaction site distances. In some embodiments, arrays of reaction sites are identical rectilinear arrays, and successive arrays in the above method are formed by printing an identical copy of the previous array, except for the movement, or shifting, of the new array's reference reaction site a predetermined distance from that of the previous array. In some embodiments, the predetermined distance is non-zero and equal to or less than the average distance between centers of adjacent reaction sites of the array. In some embodiments, the predetermined distance is in the range of from one tenth to twice the average distance between centers of adjacent reaction sites of the array. In some embodiments, the predetermined distance is solely along one axis defining the array. In some embodiments, overlapping arrays may be formed by rotating subsequent arrays, e.g., about the center of a reference reaction site, relative to a previous array. In some embodiments, steps (b) and (c) are repeated a number of times in the range of from 1 to 4, or from 1 to 3, or from 1 to 2, or steps (b) and (c) are repeated once.

### Methods of Inkjet-Assisted Template-Free Enzymatic Synthesis

Generally, methods of template-free (or equivalently, “template-independent”) enzymatic polynucleotide synthesis comprise repeated cycles of steps, in which a predetermined nucleotide is coupled to an initiator or growing chain in each cycle. The general elements of template-free enzymatic synthesis are described in the following references: Ybert et al, International patent publication WO/2015/159023; Ybert et al, International patent publication WO/2017/216472; Hyman, U.S. Pat. No. 5,436,143; Hiatt et al, U.S. Pat. No. 5,763,594; Jensen et al, Biochemistry, 57: 1821-1832 (2018); Mathews et al, Organic & Biomolecular Chemistry, DOI: 0.1039/c6ob01371f (2016); Schmitz et al, Organic Lett., 1(11): 1729-1731 (1999).

In the present invention synthesis reagents delivered by inkjet printhead nozzles must be formulated to satisfy at least two constraints: (i) the need to preserve the elongation activity of the template-free polymerase (in the case of template-free polymerase inks), and (ii) the need to meet the rheological requirements for droplet formation. The key solution parameters affecting droplet formation by inkjets are viscosity, surface tension, liquid density and the diameter of the inkjet nozzle. For particular embodiments of the invention, synthesis reagents prepared for non-droplet delivery to a reaction mixture may be reformulated by adding viscosity modifiers, surface tension modifiers and density modifiers, and the like, in order to form “printable inks” that may be delivered in droplets generated by inkjet printhead nozzles. “Printable” in reference to a reagent ink means repeatable droplets are able to be ejected from the nozzle, with uniform velocities and volumes and without satellite droplets.

In some embodiments, initiator polynucleotides with free 3′-hydroxyl groups are provided, for example, attached to a synthesis support. To the initiator polynucleotides (or elongated initiator polynucleotides in subsequent cycles) are added a 3′-O-protected-dNTP and a template-free polymerase, such as a terminal deoxynucleotidyltransferase (TdT) or variant thereof (e.g. Ybert et al, WO/2017/216472; Champion et al, WO2019/135007) under conditions effective for the enzymatic incorporation of the 3′-O-protected-dNTP onto the 3′ end of the initiator polynucleotides (or elongated initiator polynucleotides). This reaction produces elongated initiator polynucleotides whose 3′-hydroxyls are protected. If the elongated sequence is not complete, then another cycle of addition is implemented. If the elongated initiator polynucleotide contains a competed sequence, then the 3′-O-protection group may be removed, or deprotected, and the desired sequence may be cleaved from the original initiator polynucleotide. Such cleavage may be carried out using any of a variety of single strand cleavage techniques, for example, by inserting a cleavable nucleotide at a predetermined location within the original initiator polynucleotide. An exemplary cleavable nucleotide may be a uracil nucleotide which is cleaved by uracil DNA glycosylase. In accordance with some embodiments of the invention, cleavage reagents may be delivered to reaction sites in droplets generated by inkjet printhead nozzles. In such embodiments, polynucleotides at reaction sites known to be incomplete or otherwise defective can be separated from fully competed polynucleotides or can be selectively re-synthesized either by cleaving and re-synthesizing the entire polynucleotide, or by cleaving or otherwise removing incorrect sequences and re-synthesizing only the defective part of the polynucleotide.

If the elongated initiator polynucleotide is not a completed sequence (i.e. the end product), then the 3′-O-protection groups are removed to expose free 3′-hydroxyls and the elongated initiator polynucleotides are subjected to another cycle of nucleotide addition and deprotection.

In some embodiments, an initiator may comprise a non-nucleic acid compound having a free hydroxyl to which a TdT may couple a 3′-O-protected dNTP, e.g. Baiga, U.S. patent publications US2019/0078065 and US2019/0078126.

After synthesis is completed polynucleotides with the desired nucleotide sequence may be released from initiators and the synthesis supports by cleavage.

A wide variety of cleavable linkages or cleavable nucleotides may be used for this purpose. In some embodiments, cleaving the desired polynucleotide leaves a natural free 5′-hydroxyl on a cleaved strand; however, in alternative embodiments, a cleaving step may leave a moiety, e.g. a 5′-phosphate, that may be removed in a subsequent step, e.g. by phosphatase treatment. Cleaving steps may be carried out chemically, thermally, enzymatically or by photochemical methods. In some embodiments, cleavable nucleotides may be nucleotide analogs such as deoxyuridine or 8-oxo-deoxyguanosine that are recognized by specific glycosylases (e.g. uracil deoxyglycosylase followed by endonuclease VIII, and 8-oxoguanine DNA glycosylase, respectively). In some embodiments, cleavage may be accomplished by providing initiators with a deoxyinosine as the penultimate 3′ nucleotide, which may be cleaved by endonuclease V at the 3′ end of the initiator leaving a 5′-phosphate on the released polynucleotide. In some embodiments, an initiator may contain a terminal uridine so that after synthesis the desired polynucleotide may be cleaved from the initiator by treatment with KOH, or like base. Further methods for cleaving single stranded polynucleotides are disclosed in the following references, which are incorporated by reference: U.S. Pat. Nos. 5,739,386, 5,700,642 and 5,830,655; and U.S. Patent Publication Nos. 2003/0186226 and 2004/0106728; and in Urdea and Horn, U.S. Pat. No. 5,367,066.

In some embodiments, an ordered sequence of nucleotides are coupled to an initiator nucleic acid using a template-free polymerase, such as TdT, in the presence of 3′-O-protected dNTPs in each synthesis step. In some embodiments, the method of synthesizing an oligonucleotide comprises the steps of (a) providing an initiator having a free 3′-hydroxyl; (b) reacting under extension (or elongation) conditions the initiator or an extension intermediate having a free 3′-hydroxyl with a template-free polymerase in the presence of a 3′-O-protected nucleoside triphosphate to produce a 3′-O-protected extension intermediate; (c) deprotecting the extension intermediate to produce an extension intermediate with a free 3′-hydroxyl; and (d) repeating steps (b) and (c) until the polynucleotide is synthesized. (Sometimes the terms “extension intermediate” or “elongation fragment” or “growing chain” are used interchangeably). As used herein, the term “elongation conditions” means physical and chemical conditions of a reaction mixture necessary for a template-free polymerase to catalyze an elongation reaction wherein a 3′-O-protected nucleoside triphosphate monomer is coupled (by formation of a phosphodiester bond) to a free 3′-hydroxy of a nucleic acid fragment which, for example, may be an initiator or an elongated fragment. Exemplary elongation conditions include selections of reaction temperature, reaction duration, pH, concentrations of various salts, scavengers of undesired reaction components, agents to reduce nucleic acid secondary structures, and the like. In some embodiments, an initiator is provided as an oligonucleotide attached to a solid support, e.g., by its 5′ end. The above method may also include washing steps after the reaction, or extension, step, as well as after the de-protecting step. For example, the step of reacting may include a sub-step of removing unincorporated nucleoside triphosphates, e.g., by washing, after a predetermined incubation period, or reaction time. In some embodiments, such predetermined incubation periods or reaction times may be in the range of from 30 seconds to 30 minutes, or from 1 min to 30 min, or from 1 min to 15 min, or from 1 min to 10 min, or from 30 see to 5 min.

In some embodiments, after the synthesis cycles are completed, further steps may be performed to cleave the completed polynucleotides from the solid supports. Such further steps may be performed at the reaction sites of the array. Additionally, some cleavage methods may result in a released product that still requires modification to convert it into a useable product. For example, in the “endonuclease V-inosine” cleavage (described below) leaves a 5′-phosphate that must be removed for some applications. Thus, a further step of phosphatase treatment may be required.

When the predetermined sequences of polynucleotides on a synthesis support includes reverse complementary subsequences, secondary intra-molecular or cross-molecular structures may be created by the formation of hydrogen bonds between the reverse complementary regions. In some embodiments, base protecting moieties for exocyclic amines are selected so that hydrogens of the protected nitrogens cannot participate in hydrogen bonding, thereby preventing the formation of such secondary structures. That is, base protecting moieties may be employed to prevent the formation of hydrogen bonds, such as are formed in normal base pairing, for example, between nucleosides A and T and between G and C. At the end of a synthesis, the base protecting moieties may be removed and the polynucleotide product may be cleaved from the solid support, for example, by cleaving it from its initiator.

In addition to providing 3′-O-blocked dNTP monomers with base protection groups, elongation reactions may be performed at higher temperatures using thermal stable template-free polymerases. For example, a thermal stable template-free polymerase having activity above 40° C. may be employed; or, in some embodiments, a thermal stable template-free polymerase having activity in the range of from 40-85° C. may be employed; or, in some embodiments, a thermal stable template-free polymerase having activity in the range of from 40-65° C. may be employed.

In some embodiments, elongation conditions may include adding solvents to an elongation reaction mixture that inhibit hydrogen bonding or base stacking. Such solvents include water miscible solvents with low dielectric constants, such as dimethyl sulfoxide (DMSO), methanol, and the like. Likewise, in some embodiments, elongation conditions may include the provision of chaotropic agents that include, but are not limited to, n-butanol, ethanol, guanidinium chloride, lithium perchlorate, lithium acetate, magnesium chloride, phenol, 2-propanol, sodium dodecyl sulfate, thiourea, urea, and the like. In some embodiments, elongation conditions include the presence of a secondary-structure-suppressing amount of DMSO. In some embodiments, elongation conditions may include the provision of DNA binding proteins that inhibit the formation of secondary structures, wherein such proteins include, but are not limited to, single-stranded binding proteins, helicases, DNA glycolases, and the like.

When base-protected dNTPs are employed, the above method may further include a step (e) removing base protecting moieties, which in the case of acyl or amidine protection groups may (for example) include treating with concentrated ammonia.

The above method may also include capping step(s) as well as washing steps after the reacting, or extending, step, as well as after the deprotecting step. As mentioned above, in some embodiments, capping steps may be included in which non-extended free 3′-hydroxyls are reacted with compounds that prevents any further extensions of the capped strand. In some embodiments, such compound may be a dideoxynucleoside triphosphate. In other embodiments, non-extended strands with free 3′-hydroxyls may be degraded by treating them with a 3′-exonuclease activity, e.g. Exo I. For example, see Hyman, U.S. Pat. No. 5,436,143. Likewise, in some embodiments, strands that fail to be deblocked may be treated to either remove the strand or render it inert to further extensions. When a capping agent, such as ddNTPs, are used, the buffer or synthesis reagents containing such agents may be delivered by flowing or spraying such reagent over substrate containing the reaction sites.

In some embodiments, reaction conditions for an elongation step (also sometimes referred to as an extension step or a coupling step) may comprising the following: 20 μM purified TdT; 125-600 μM 3′-O-blocked dNTP (e.g. 3′-O—NH₂-blocked dNTP); about 10 to about 500 mM potassium cacodylate buffer (pH between 6.5 and 7.5) and from about 0.01 to about 10 mM of a divalent cation (e.g. CoCl₂ or MnCl₂), where the elongation reaction may be carried out at a temperature within the range RT to 45° C., for 3 minutes. It is understood that whenever the foregoing coupling reagent is delivered by inkjet-produced droplets its viscosity, density and surface tension must be adjusted so that it becomes a printable ink. In this connection, the invention in part includes the recognition and appreciation that an ink for delivering TdT to a reaction site may have its viscosity modified for droplet formation and activity preserved by selection of a viscosity modifier, such as, when carboxymethyl cellulose is selected as the viscosity modifying agent.

In some embodiments, in which the 3′-O-blocked dNTPs are 3′-O—NH₂-blocked dNTPs, reaction conditions for a deblocking step may comprise the following: 700 mM NaNO₂; 1 M sodium acetate (adjusted with acetic acid to pH in the range of 4.8-6.5), where the deblocking reaction may be carried out at a temperature within the range of RT to 45° C. for 30 seconds to several minutes. Washes may be performed with the cacodylate buffer without the components of the coupling reaction (e.g., enzyme, monomer, divalent cations). If the above reagent compositions are delivered to reaction sites by inkjet delivery, it is understood that the compositions would be altered to meet the rheological requirements for droplet formation by the nozzles of the inkjet print heads used.

In some embodiments, RNA synthesis may be accomplished by similar steps as described above but with template-free polymerases and monomers specifically selected for RNA synthesis, such as, polyA polymerase (PAP), polyU polymerase (PUP), or the like, e.g. International patent publication WO2020/077227. For example, systems, apparatus and kits of the invention may implement methods of synthesizing a polyribonucleotide having a predetermined sequence comprising the steps of: a) providing an initiator having a 3′-terminal nucleotide having a free 3′-hydroxyl; and b) repeating, until the polyribonucleotide is formed, cycles of (i) contacting under elongation conditions the initiator or elongated fragments having free 3′-hydroxyls with a 3′-O-blocked-nucleoside triphosphate and a template-free polymerase so that the initiator or elongated fragments are elongated by incorporation of a 3′-O-blocked-nucleoside triphosphate to form 3′-O-blocked-elongated fragments, and (ii) deblocking the elongated fragments to form elongated fragments having free 3′-hydroxyls; wherein the template-free polymerase is a poly(A) polymerase (PAP) or a poly(U) polymerase. In further embodiments, the initiator may be attached to a support by a 5′ end, the support may be a solid support, and the above method may include a step of cleaving the polynucleotide from the initiator. In some embodiments, reaction conditions for an extension or elongation step using PAP or PUP may comprising the following: Reaction conditions 1 (for primer+AM-rATP): 250 uM AM-rATP, 0.1 uM ATT0488-(rA)5, 1 uM PAP, 1×ATP buffer (20 mM Tris-HCl, 0.6 mM MnCl2, 0.02 mM EDTA, 0.1% BSA, 10% glycerol, 100 mM imidazole, pH 7-8), 37 C, 30 min. Reaction condition 2 (for primer+AM-rGTP): 250 uM rGTP, 0.1 uM ATT0488-(rA)5, 1 uM PAP, 1×GTP buffer (0.6 mM MnCl2, 0.1% BSA, 10 mM imidazole, pH 6), 37 C, 30 min. In the foregoing, “AM-rNTP” refers to 3′-azidomethyl-O-ribonucleoside triphosphate. Many of the 3′-O-blocked rNTPs employed in the invention may be purchased from commercial vendors (e.g. Jena Bioscience, MyChemLabs, or the like) or synthesized using published techniques, e.g., U.S. Pat. No. 7,057,026; International patent publications WO2004/005667, WO91/06678; Canard et al, Gene (cited above); Metzker et al, Nucleic Acids Research, 22: 4259-4267 (1994); Meng et al, J. Org. Chem., 14: 3248-3252 (3006); U.S. patent publication 2005/037991; Zavgorodny et al, Tetrahedron Letters, 32(51): 7593-7596 (1991). In a further particular embodiment, the 3′-blocked nucleotide triphosphate is blocked by either 3′-O-propargyl, a 3′-O-azidomethyl, 3′-O—NH₂ or 3′-O-allyl group. In still other embodiments, 3′-O-blocking groups of the invention include 3′-O-methyl, 3′-O-(2-nitrobenzyl), 3′-O-allyl, 3′-O-amine, 3′-O-azidomethyl, 3′-O-tert-butoxy ethoxy, 3′-O-(2-cyanoethyl), and 3′-O-propargyl. As above, if the above reagent compositions are delivered to reaction sites by inkjet delivery, it is understood that the compositions would be altered to meet the rheological requirements for droplet formation by the nozzles of the inkjet print heads used.

### 3′-O-Protected Nucleoside Triphosphates

Depending on particular applications, the steps of deblocking and/or cleaving may include a variety of chemical or physical conditions, e.g., light, heat, pH, presence of specific reagents, such as enzymes, which are able to cleave a specified chemical bond. Guidance in selecting 3′-O-blocking groups and corresponding de-blocking conditions may be found in the following references, which are incorporated by reference: Benner, U.S. Pat. Nos. 7,544,794 and 8,212,020; 5,808,045; 8,808,988; International patent publication WO91/06678; and references cited below. In some embodiments, the cleaving agent (also sometimes referred to as a de-blocking reagent or agent) is a chemical cleaving agent, such as, for example, dithiothreitol (DTT). In alternative embodiments, a cleaving agent may be an enzymatic cleaving agent, such as, for example, a phosphatase, which may cleave a 3′-phosphate blocking group. It will be understood by the person skilled in the art that the selection of deblocking agent depends on the type of 3′-nucleotide blocking group used, whether one or multiple blocking groups are being used, whether initiators are attached to living cells or organisms or to solid supports, and the like, that necessitate mild treatment. For example, a phosphine, such as tris(2-carboxyethyl)phosphine (TCEP) can be used to cleave a 3′O-azidomethyl groups, palladium complexes can be used to cleave a 3′O-allyl groups, or sodium nitrite can be used to cleave a 3′O-amino group. In particular embodiments, the cleaving reaction involves TCEP, a palladium complex or sodium nitrite.

As noted above, in some embodiments it is desirable to employ two or more blocking groups that may be removed using orthogonal de-blocking conditions. The following exemplary pairs of blocking groups may be used in parallel synthesis embodiments. It is understood that other blocking group pairs, or groups containing more than two, may be available for use in these embodiments.

| | |
|---|---|
| 3′-O-NH2 | 3′-O-azidomethyl |
| 3′-O-NH2 | 3′-O-allyl, 3′-O-propargyl |
| 3′-O-NH2 | 3′-O-phosphate |
| 3′-O-azidomethyl | 3′-O-allyl, 3′-O-propargyl |
| 3′-O-azidomethyl | 3′-O-phosphate |
| 3′-O-allyl, 3′-O-propargyl | 3′-O-phosphate |

In some embodiments, specific enzymatically removable blocking groups are used with specific enzymes for their removal. For example, ester- or acyl-based blocking groups may be removed with an esterase, such as acetylesterase, or like enzyme, and a phosphate blocking group may be removed with a 3′ phosphatase, such as T4 polynucleotide kinase. By way of example, 3′-O-phosphates may be removed by treatment with as solution of 100 mM Tris-HCl (pH 6.5) 10 mM MgCl₂, 5 mM 2-mercaptoethanol, and one Unit T4 polynucleotide kinase. The reaction proceeds for one minute at a temperature of 37° C. As above, if the foregoing compositions are delivered to reaction sites by inkjet delivery, it is understood that the compositions would be altered to meet the rheological requirements for droplet formation by the nozzles of the inkjet print heads used.

Further examples of synthesis and enzymatic deprotection of 3′-O-ester-protected dNTPs or 3′-O-phosphate-protected dNTPs are described in the following references: Canard et al, Proc. Natl. Acad. Sci., 92:10859-10863 (1995); Canard et al, Gene, 148: 1-6 (1994); Cameron et al, Biochemistry, 16(23): 5120-5126 (1977); Rasolonjatovo et al, Nucleosides & Nucleotides, 18(4&5): 1021-1022 (1999); Ferrero et al, Monatshefte fur Chemie, 131: 585-616 (2000); Taunton-Rigby et al, J. Org. Chem., 38(5): 977-985 (1973); Uemura et al, Tetrahedron Lett., 30(29): 3819-3820 (1989); Becker et al, J. Biol. Chem., 242(5): 936-950 (1967); Tsien, International patent publication WO1991/006678.

In some embodiments, the modified nucleotides comprise a modified nucleotide or nucleoside molecule comprising a purine or pyrimidine base and a ribose or deoxyribose sugar moiety having a removable 3′-OH blocking group covalently attached thereto, such that the 3′ carbon atom has attached a group of the structure:

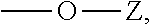

wherein —Z is any of —C(R′)₂—O—R″, —C(R′)₂—N(R″)₂, —C(R′)₂—N(H)R″, —C(R′)₂—S—R″ and —C(R′)₂—F, wherein each R″ is or is part of a removable protecting group; each R′ is independently a hydrogen atom, an alkyl, substituted alkyl, arylalkyl, alkenyl, alkynyl, aryl, heteroaryl, heterocyclic, acyl, cyano, alkoxy, aryloxy, heteroaryloxy or amido group, or a detectable label attached through a linking group; with the proviso that in some embodiments such substituents have up to 10 carbon atoms and/or up to 5 oxygen or nitrogen heteroatoms; or (R′)₂ represents a group of formula ═C(R′″)₂ wherein each R′″ may be the same or different and is selected from the group comprising hydrogen and halogen atoms and alkyl groups, with the proviso that in some embodiments the alkyl of each R′″ has from 1 to 3 carbon atoms; and wherein the molecule may be reacted to yield an intermediate in which each R″ is exchanged for H or, where Z is —(R′)₂—F, the F is exchanged for OH, SH or NH₂, preferably OH, which intermediate dissociates under aqueous conditions to afford a molecule with a free 3′-OH; with the proviso that where Z is —C(R′)₂—S—R″, both R′ groups are not H. In some embodiments, R′ of the modified nucleotide or nucleoside is an alkyl or substituted alkyl, with the proviso that such alkyl or substituted alkyl has from 1 to 10 carbon atoms and from 0 to 4 oxygen or nitrogen heteroatoms. In some embodiments, —Z of the modified nucleotide or nucleoside is of formula —C(R′)₂—N3. In some embodiments, Z is an azidomethyl group.

In some embodiments, Z is a cleavable organic moiety with or without heteroatoms having a molecular weight of 200 or less. In other embodiments, Z is a cleavable organic moiety with or without heteroatoms having a molecular weight of 100 or less. In other embodiments, Z is a cleavable organic moiety with or without heteroatoms having a molecular weight of 50 or less. In some embodiments, Z is an enzymatically cleavable organic moiety with or without heteroatoms having a molecular weight of 200 or less. In other embodiments, Z is an enzymatically cleavable organic moiety with or without heteroatoms having a molecular weight of 100 or less. In other embodiments, Z is an enzymatically cleavable organic moiety with or without heteroatoms having a molecular weight of 50 or less. In other embodiments, Z is an enzymatically cleavable ester group having a molecular weight of 200 or less. In other embodiments, Z is a phosphate group removable by a 3′-phosphatase. In some embodiments, one or more of the following 3′-phosphatases may be used with the manufacturer's recommended protocols: T4 polynucleotide kinase, calf intestinal alkaline phosphatase, recombinant shrimp alkaline phosphatase (e.g. available from New England Biolabs, Beverly, MA)

In some embodiments, the 3′-blocked nucleotide triphosphate is blocked by either a 3′-O-azidomethyl, 3′-O—NH₂ or 3′-O-allyl group.

In some embodiments, 3′-O-blocking groups of the invention include 3′-O-methyl, 3′-O-(2-nitrobenzyl), 3′-O-allyl, 3′-O-amine, 3′-O-azidomethyl, 3′-O-tert-butoxy ethoxy, 3′-O-(2-cyanoethyl), and 3′-O-propargyl.

3′-O-blocked dNTPs without base protection may be purchased from commercial vendors or synthesized using published techniques, e.g. U.S. Pat. No. 7,057,026; Guo et al, Proc. Natl. Acad. Sci., 105(27): 9145-9150 (2008); Benner, U.S. Pat. Nos. 7,544,794 and 8,212,020; International patent publications WO2004/005667, WO91/06678; Canard et al, Gene (cited herein); Metzker et al, Nucleic Acids Research, 22: 4259-4267 (1994); Meng et al, J. Org. Chem., 14: 3248-3252 (2006); U.S. patent publication 2005/037991. 3′-O-blocked dNTPs with base protection may be synthesized as described below.

Aspects, including embodiments, of the present subject matter described above may be beneficial alone or in combination, with one or more other aspects or embodiments. Without limiting the foregoing description, certain non-limiting aspects of the disclosure numbered 1-91 are provided below. As will be apparent to those of skill in the art upon reading this disclosure, each of the individually numbered aspects may be used or combined with any of the preceding or following individually numbered aspects.

### EXAMPLES

The following examples are put forth so as to provide those of ordinary skill in the art with a complete disclosure and description of how to make and use the disclosed subject matter, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. It should be understood, however, that the intention is not to limit the invention to the particular embodiments described. The intention is to cover all modifications, equivalents, and alternatives falling within the spirit and scope of the invention.

### Example 1: Stable Formulations for Inkjet Enzymatic DNA Synthesis

For successful inkjet enzymatic DNA synthesis, the buffer containing the enzyme and nucleotide (referred to as an “ink”) is preferably active (i.e., result in efficient chain extension); stable (i.e., does not form a precipitate that may clog the printhead); and printable (i.e., has appropriate viscosity (η) and surface tension (γ)).

Here we define active and stable as sufficient for a whole synthesis run lasting 300 cycles and 3-4 days. Preferably, the ink can be used for multiple runs because removing ink from printheads and loading printheads with fresh ink is a difficult and time-consuming process. Furthermore, fluid reservoirs, fluid delivery systems and printheads typically have volumes (>milliters) that far exceed the consumption of ink in a typical synthesis run (inkjet printheads only deliver picoliter (1-30) volumes of reagent to each location (typically 30 microns in diameter) on the substrate and the substrate is typically small (10 cm²-1 m²)). Not having to dispose of unused ink after each synthesis run helps, therefore, to reduce cost.

Standard single inks found in the literature usually contain cacodylate (or another equivalent buffer e.g., HEPES, MES) and cobalt (or another Group 4 transition metal e.g., Ni, Mn, Zn) as well as a nucleotide (usually 3′unblocked) and terminal deoxynucleotide enzyme (TdT). They are usually active but are unprintable, lacking the necessary surface tension and viscosity for ejection of discrete droplets without satellites. Most are unstable forming a precipitate in a matter of hours. Some replace the transition metal with an alkali or alkali earth metal (e.g., magnesium), which may be more stable but may lack the necessary activity for EDS. Furthermore, standard single inks typically do not contain nucleotides with reversible terminators; therefore, such inks do not provide single-base control.

As part of developing an inkjet EDS platform we have identified the causes of ink instability, discovered stable versions of inks, optimized storage conditions; and identified different ways the ink can be used. The following factors are important to produce an active, stable ink: the final ink pH and the cofactor concentration. The ink is preferably cooled at −20° C. to 4° C. during storage off printer, and stored as cool as permitted by the manufacturer of the fluid delivery system, printhead and the cooling system in use when used on printer. The ink may be split into two and the two inks printed separately. The preferred ink combinations are (enzyme, cobalt)+(nucleotide, DMSO), or, (enzyme, nucleotide)+(cobalt, DMSO). The ink(s) should be filtered.

Table 1 provides information on a typical standard ink (‘Standard’) and for comparison, an ink that at 20° C. is greater than 20 times more stable and retains its original activity (‘Optink0.25’). The results of the experimental work described further below show how stability and activity are impacted by the formulation.

TABLE 1

| COMPONENTS | | | VISUAL STABILITY | | FORMULATIONS | | | |
|---|---|---|---|---|---|---|---|---|
| Category | Role | Standard | Impact | Improvement | Standard | | Optink0.25 | |
| EDS | Nucleotide | 3′ONH₂- | 5/5 | — | 500 | μM | 500 | μM |
| Active | | dNTP | | | | | | |
| | Enzyme | TdT | 5/5 | No thaw/freeze | 20 | μM | 20 | μM |
| | | | | cycles | | | | |
| | Cofactor | CoCl₂ | 5/5 | <[CoCl₂]; Use | 1 | mM | 0.25 | mM |
| | | | | MgCl₂ | | | | |
| Denaturant | Reduce Tm | DMSO | 5/5 | <[DMSO] | 15% | | 15% | |
| Buffer | Control pH | Cacodylic | — | — | 0.5M | | 0.5M | |
| | | acid | | | | | | |
| | | pH | 5/5 | Final pH < 6.5 | Final pH = 6.6^{a)} | | Final pH = 6.0^{a)} | |
| EDS | η modifier | Glycerol | 4/5 | <[Gly] | 10-20% | | 10% | |
| Inactive | γ modifier | Tween20 | 2/5 | Presence | 0.05% | | 0.05% | |
| | Scavenger | BOX•HCl | 2/5 | — | 50 | mM | 50 | mM |

^{a)}pH measured for DMSO-free solutions as DMSO impacts the reading.

### Example 2: Causes of Instability

‘Standard’ ink (Table 1) containing nucleotide, cobalt, cacodylate buffer and enzyme was prepared and stored in 5 mL Eppendorfs tubes and stored in the dark at either 22° C. or 4° C. The inks were prepared by adding the components in the following order: MQ-water, glycerol, Tween 20, cacodylate (2 M stock), BOX-HCl (250 mM stock), cobalt chloride (100 mM stock), DMSO, nucleotide (10 mM stock), enzyme (200 μM). Mixing was performed after adding the Tween 20, DMSO and enzyme. Glycerol and Tween 20 were used as a viscosity modifier and a surface tension modifier, respectively, to make the ink printable. DMSO was used to reduce the melting temperature of DNA and any secondary structure. BOX-HCl was used as an aldehyde/ketone scavenger. The nucleotide had a 3′aminoxy (ONH₂) protecting group to enable single-base extensions and the enzyme was an engineered terminal deoxynucleotidyl transferase (TdT) able to accommodate the 3′ONH₂ nucleotide as a substrate.

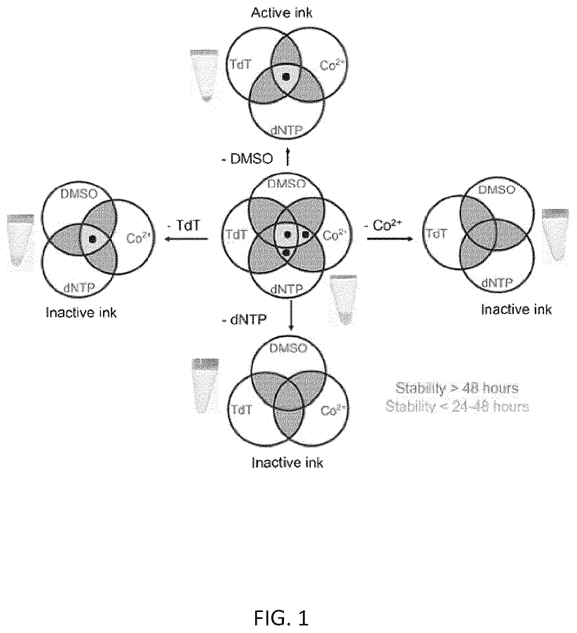

Visual stability with storage time was analyzed by periodically taking pictures of inks on a bright white background. Ink stability was also studied for inks with a single component missing. FIG. 1 shows the combinations of components that were stable and unstable. In dark gray (without black dots) are inks that were visually stable for >48 h at 22° C. In light gray (with black dots) are inks that were stable <48 h at 22° C. Inks that were active but unstable forming precipitates possessed nucleotide, cobalt, and TdT and/or DMSO. DMSO-free inks and TdT-free inks formed precipitates in <48 hours, while dNTP-free and cobalt-free inks were visually stable for >>48 h. Inks not having nucleotide or cobalt are inactive.

### Example 3: Identifying the Optimum pH and Cofactor Concentration

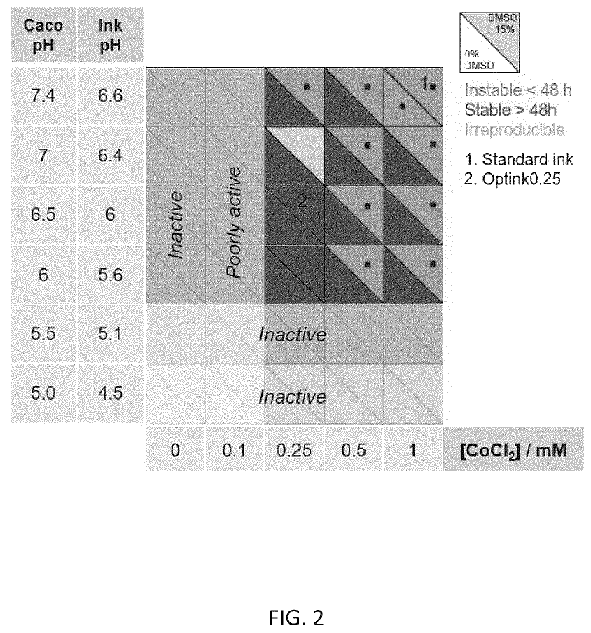

‘Standard’ ink was prepared as in Example 2 but at a lower cobalt concentration and at a lower final ink pH. The pH was modified by adjusting the pH of the cacodylate buffer stock used to prepare the ink. Visual stability with storage time was analyzed by periodically taking pictures of inks on a bright white background. FIG. 2 shows how ink instability (marked with a black dot) depends on pH and [Co], with and without DMSO. FIG. 2 shows that the most effective way to prevent precipitation is to concurrently lower the ink pH (here measured for a DMSO-free analogue) and lower the [Co]. From this study, the combination of using 0.25 mM cobalt and an ink pH of 6.0 was selected (=‘Optink0.25’), as initial activity tests indicated that the enzyme lost activity when the ink pH was below 5.1 but increased at higher pH.

### Example 4: Effects of the Nucleotide and Temperature

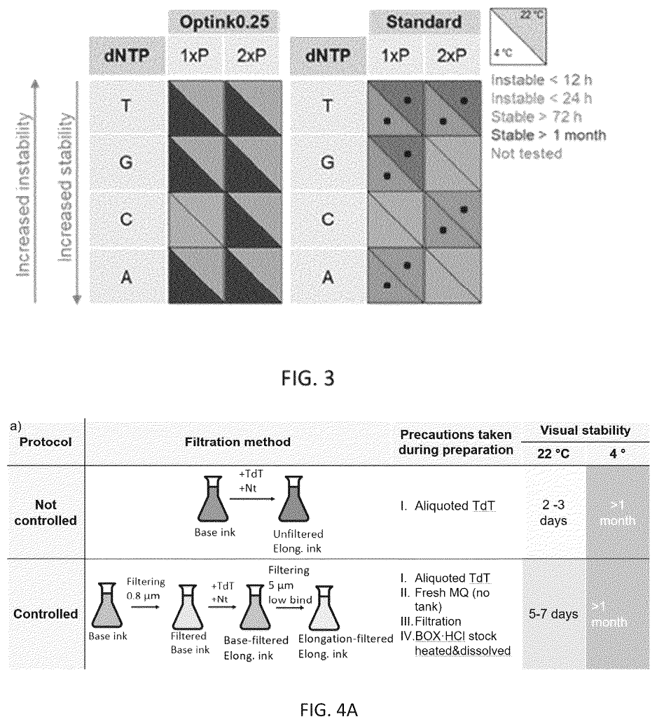

‘Optink0.25’ and ‘Standard’ ink were prepared as in Examples 2 and 3 for each nucleotide (T vs. G, C, A) and after different levels of nucleotide purification (single purified: 1×P; and double or triple purified: 2×P; Note that C was only available as triple purified). Stability was studied as in Examples 2 and 3 as a function of storage temperature (22° C. or 4° C.). The results are reported in FIG. 3. Black dots denote solutions that were unstable. Storing ink at lower temperature has a more obvious effect on stability for ‘Optink0.25’ than for ‘Standard’ ink. Inks at 4° C. were stable for greater than 1 month, which is sufficient for potentially 10 synthesis runs. Although much less impactful than temperature, the nucleotide also has an impact on stability. Ink stability was found to be in the order T<G<A≈C. For ‘Standard’ ink at 22° C., T and G precipitate in <12 hours while C and A in <24 h. For ‘Optink0.25’, T and G precipitate approximately a couple of days before C and A (i.e., 5 vs. 7 days). The purification state of the nucleotide has a marginal effect on stability. For ‘Standard’ ink with DMSO, 2×P inks precipitate only a few hours (<24 h) after 1×P inks (<6 h) using 3′-ONH2-dTTP nucleotide. For Optink0.25, 2×P inks precipitate 2 to 3 days after 1×P inks.

### Example 5: Effects of Filtering

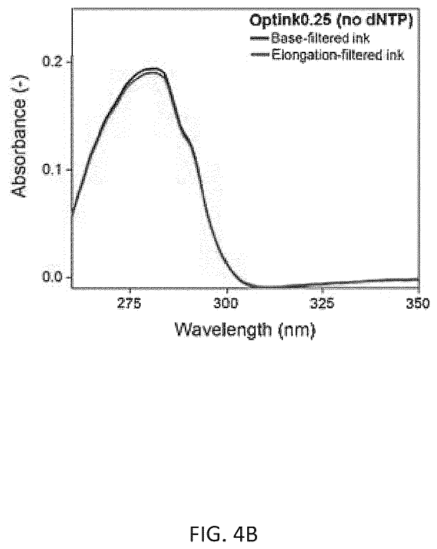

Filtration of inks before inkjet printing is standard practice and was investigated here to determine the effect on stability and activity. Adsorption of enzyme to the filter is a possibility and might result in reduced activity. Filtered inks were filtered twice: once with a 0.8 μm filter before adding enzyme and nucleotide, then with a 5 μm PTFE low bind filter after adding enzyme and nucleotide (FIG. 4A). UV-Vis measurements were made without nucleotide to facilitate quantification of TdT and after dilution, to lower the absorption below 1.0 (FIG. 4B).

Stability at 22° C. was better for filtered ink than for non-filtered ink. No difference was observed for inks stored at 4° C. UV-Vis measurements demonstrated that the filtration protocol lowered [TdT] only very slightly (0.5-2.7% depending on the volume of ink filtered).

### Example 6: Run Activity

Ink activity was tested by performing either manual EDS on DNA-functionalized glass slides, or automated EDS using a liquid handler (Tecan) on DNA-functionalized beads. DNA was photocleaved or cleaved enzymatically and submitted for gel electrophoresis. (Note, the method of enzymatic cleavage used gave two bands corresponding to a terminal phosphate or hydroxyl).

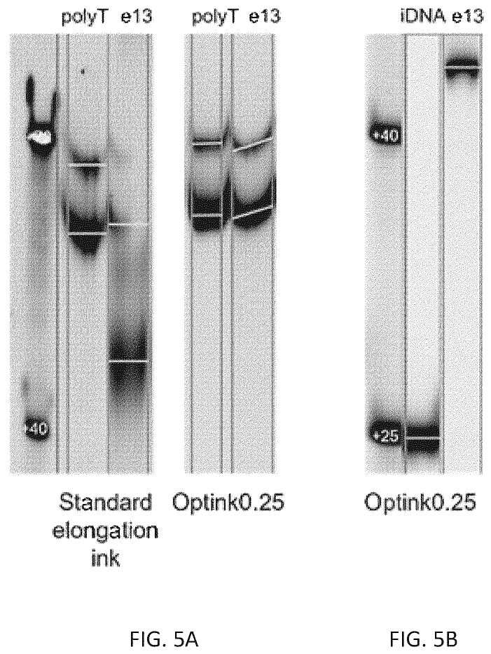

In the case of long synthesis (>5 h, >15 cycles), ‘Standard’ ink begins to precipitate before synthesis is complete resulting in lower DNA purity. FIGS. 5A-5B show the results of automated synthesis (FIG. 5A) and manual synthesis (FIG. 5B). A shorter length product band and smear is evident in the case of the 52mer synthesis of sequence e13 with ‘Standard’ ink. This smear is not present for the case of ‘Optink0.25’ or for the case of a shorter (21 cycle) manual synthesis with ‘Optink0.25’.

### Example 7: Activity Versus Storage Temperature

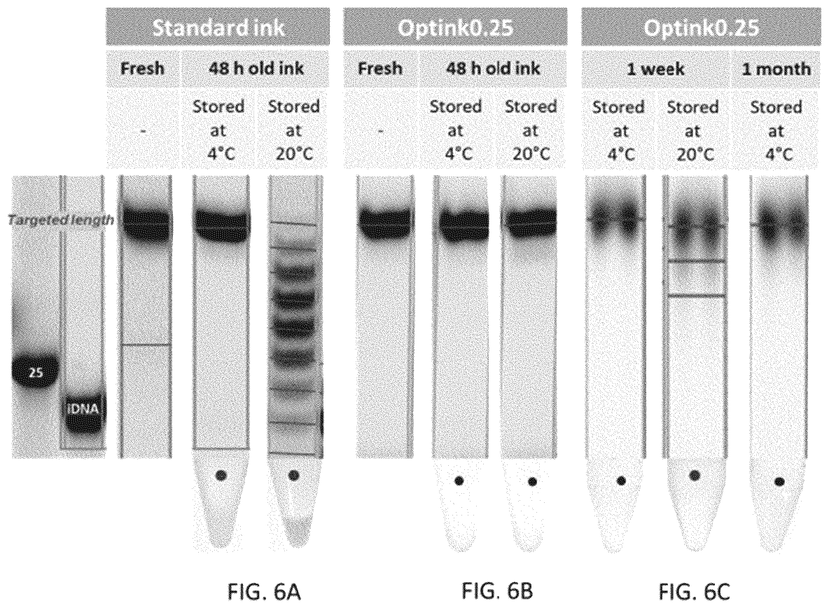

Manual synthesis was performed on ‘Standard’ ink and ‘Optink0.25’ to test for activity after aging at 4° C. and 20° C. After 48 hours, ‘Standard’ ink precipitated at both storage temperatures (FIG. 6A). However, the supernatant of ‘Standard’ ink stored at 4° C. was still active while the supernatant of ‘Standard’ ink stored at 20° C. gave a series of bands at shorter length, corresponding to poor purity.

By comparison, ‘Optink0.25’, was visually stable at both storage temperatures and gave good purity for greater than 1 month if stored at 4° C. At 20° C., stability was also much greater, up to almost 1 week (FIGS. 6B-6C). The experiment highlights the importance of storage temperature for conserving activity and how the relationship between stability and activity is not parity.

### Example 8: HPLC Monitoring of Nucleotide Concentration

Ion exchange IE-HPLC-UV studies were performed to monitor the decay of 3′ONH₂-dNTP (N=T or G) in the ink upon aging. ‘Standard’ ink, ‘Optink0.25’, and a control ink (i.e., Optink0.25 without CoCl₂ and TdT) were prepared, aged at either 20 or 4° C., and filtered through a centrifugal Amicon filter (3 kDa) to remove TdT.

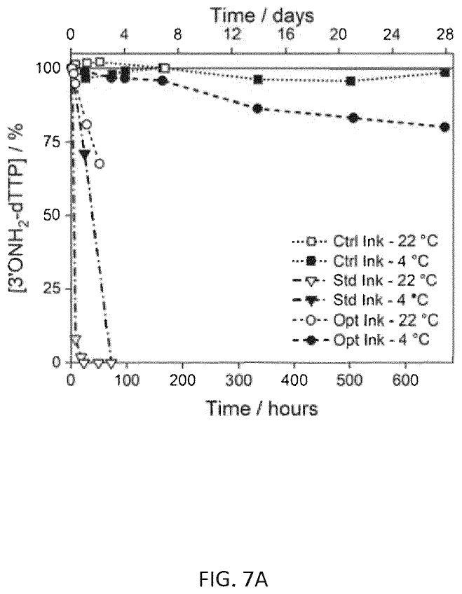

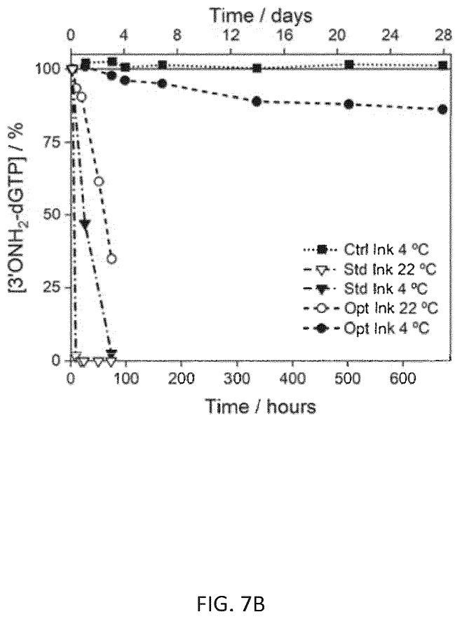

The results are summarized in FIGS. 7A-7B. Nucleotide in control ink (ink lacking cobalt and TdT) was perfectly stable at both storage temperatures. ‘Standard’ inks aged at 20° C. and 4° C., however, showed a rapid decay of both dTTP and dGTP. ‘Optink0.25’ stored at 20° C., by comparison, showed a significantly slower loss of dNTP, particularly when stored at 4° C. (>80% remaining after 4 weeks). An even slower degradation profile would be expected at lower temperatures. Therefore, ‘Optink0.25’ was tested to see if it withstood typical freezer temperatures. Due to the presence of glycerol and DMSO in the ink and freezing point depression, the ink remained a liquid at −20° C. At −78° C., the ink is expected to freeze but upon thawing the ink may be fully functional (not tested).

### Example 9: Role of Pyrophosphate

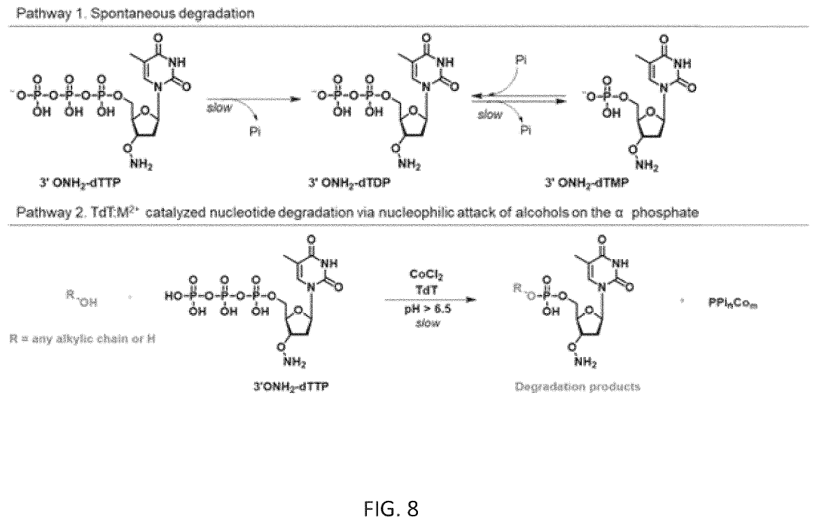

The above Examples show that precipitation occurs upon aging but can be delayed by cooling, lowering the pH<6.5 and/or by lowering the [CoCl₂]<1.0 mM. Adding inorganic pyrophosphatase (PPase) was also helpful in preventing precipitation suggesting pyrophosphate (PPi) is involved in the precipitation mechanism. How it is formed is unclear, but the precipitate may form by complexation of pyrophosphate with cobalt and other ligands (L). This hypothesis is supported by double purified nucleotides (2×P) giving slightly more stable inks than single purified nucleotides (1×P). We speculate that above pH 6.5 (≈pKa of dNTP) and at higher cobalt concentrations complexation is favored resulting in faster precipitation. We also speculate that the pyrophosphate is generated in situ. Normally, dNTPs degrade slowly in aqueous solution through consecutive steps of single dephosphorylation. This is shown in FIG. 8 (pathway 1). In a complex aqueous solution, however, another pH-dependent pathway involving coordination by transition metal ions (M²⁺, such as Co²⁺) and nucleophilic attack at the alpha position phosphate may be possible (pathway 2). This would generate pyrophosphate, whose formation might also inhibit chain extension as in the case of PCR reactions.

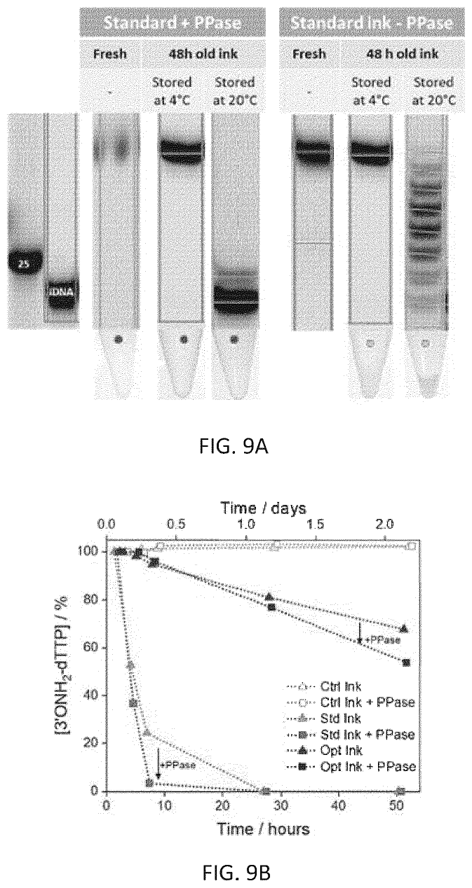

To test if removing or lowering pyrophosphate in solution would prevent precipitation, inorganic pyrophosphatase ([2.3 nM]) was added to ‘Standard’ and ‘Optink0.25’. PPase catalyzes the hydrolysis of pyrophosphate to phosphate. As FIG. 9A shows, addition of PPase increased the visual stability of the inks confirming that precipitate formation is PPi driven. Addition of PPase to the ink is beneficial therefore if the aim is simply to reduce the risk of the printhead becoming blocked. It does not help, however, in terms of ink activity. This is likely due to the fact that, by consuming pyrophosphate, PPase promotes pathway 2, which in turn hastens the consumption of the nucleotide (3′ONH₂-dNTP). This hypothesis is supported by IE-HPLC-UV (FIG. 9B).

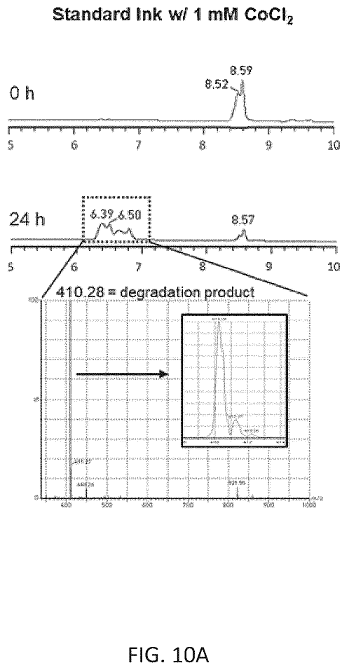

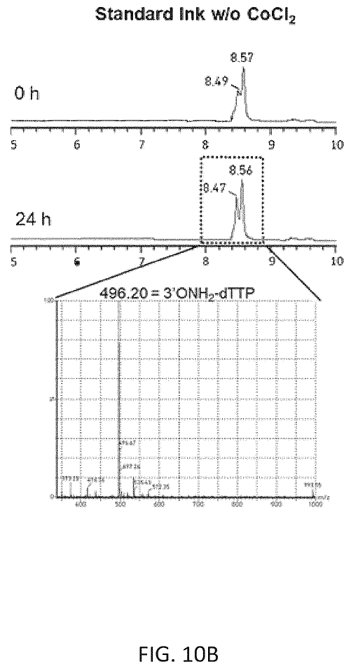

To further corroborate the hypothesis that cobalt also plays an active part in the degradation process HPLC-MS measurements were performed. ‘Standard’ ink showed a rapid 3′ONH₂-dNTP degradation profile in the presence of cobalt (FIG. 10A) and a constant [3′ONH₂-dNTP] in its absence (FIG. 10B).

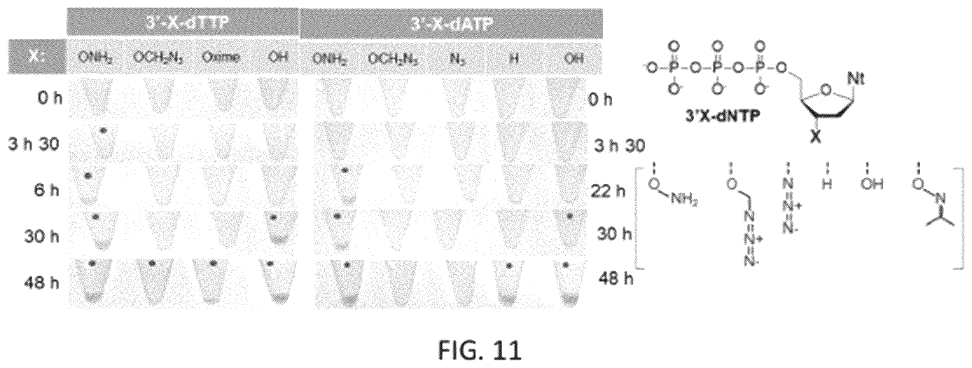

To gain further insights, stability studies were performed on ‘Standard’ ink formulated with 3′X-dNTP with different 3′ functionalization (N=A, T; X=—ONH₂ (2×P), —OCH₂N₃, —N₃, acetone oxime, —OH, —H). The aminoxy group (ONH₂) is a good nucleophile and might contribute to the formation of pyrophosphate and precipitation. Indeed, 3′ONH₂-dNTP ‘Standard’ ink precipitated fastest when stored at 20° C. followed by ‘Standard’ ink containing 3′OH-dNTP. Additionally, ‘Standard’ ink containing dTTP always precipitated faster than dATP analogues, irrespective of 3′ functionalization (FIG. 11). We speculate that this is due to the capacity of the nucleobase to complex cobalt and participate in precipitation.

### Example 10: Splitting of Inks

Based on the results of Example 2 and learning from subsequent experiments we reasoned that separating ink components and printing them separately in the same location may be beneficial in terms of storage stability and activity. To avoid printing too many inks, we chose to test only ‘two-pack’ combinations and combinations where cobalt and nucleotide are separated. Printing every component separately would take more time, require additional fluid reservoirs and printheads and introduce potential errors through imprecise alignment of the different inks at a given location.

A split ink comprising 2×TdT-2×CoCl₂ (1A) and 2×Nt-2×DMSO (1B) and a split ink containing 2×TdT-2×Nt (2A) and 2×CoCl₂-2×DMSO (2B) and a split ink containing 3×Nt-2×CoCl₂-2.6×DMSO (3A) and 2.5×TdT (3B) were prepared. Inks were filtered (0.8 μm) before adding TdT and 3′-ONH₂-dNTP (N=T) and their stability at 22° C. assessed. Mixed in a 1:1 v/v ratio the split inks (parts 1A with 1B or 2A with 2B or 3A with 3B) would give ‘Standard’ or ‘Optink0.25’ ink.

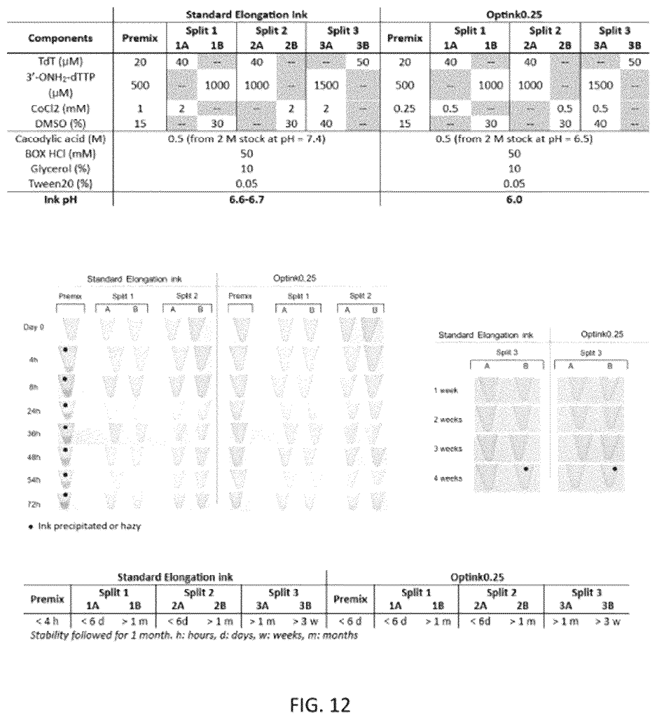

As shown in FIG. 12, ‘Standard’ ink (combined ink, here called ‘Premix’) is the worst for stability and the split ink version of ‘Standard’ ink is on a par with ‘Optink0.25’combined and ‘Optink0.25’ split ink in terms of stability. ‘Optink0.25’ ink (combined), however, is the best ink because it can be printed in one step.

### Example 11: Activity of the Ink as a Function of pH

For the following examples, the term elongation ink refers to an ink according to the values of Optink0.25 of Table 1 for nucleotide, enzyme, denaturant, 1 modifier, 7 modifier and scavenger. The cofactor, buffer and pH can change and are indicated in the example.

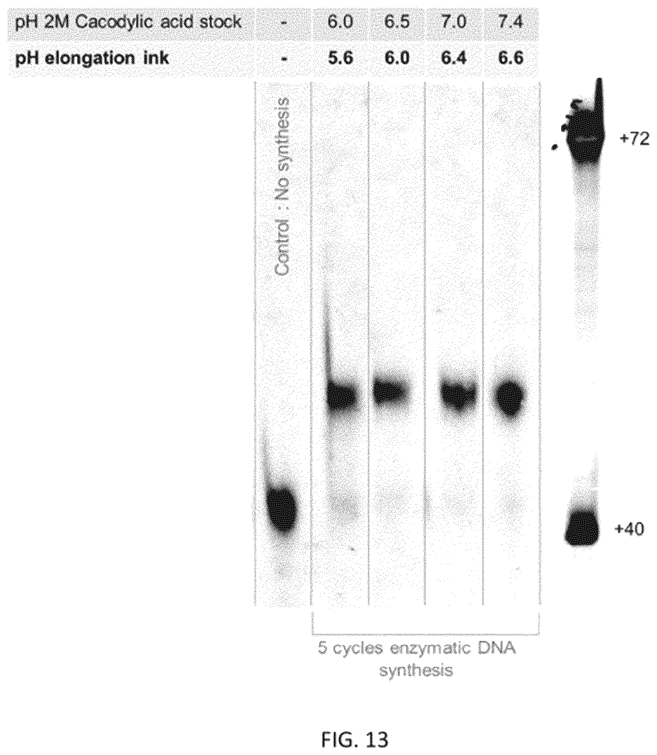

Manual synthesis was performed with the elongation ink for 5 cycles to test for the ink activity at different pHs. As shown in FIG. 13, the activity is preserved in the pH range between 5.6 and 6.6.

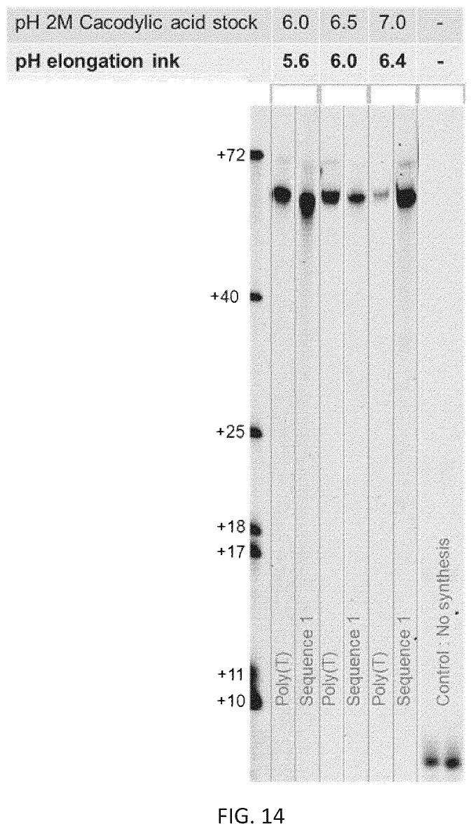

Further, an automated synthesis was performed using the elongation ink for 52 cycles of Poly(T) and Sequence 1 which is a synthetic sequence of 52 nucleotides with all 4 bases without secondary structure and G quadruplex to test the ink activity at different pHs for longer sequences. As shown in FIG. 14, the ink activity is satisfactorily preserved at pHs between 5.6 and 6.4.

### Example 12: Activity of the Ink as a Function of [Co]

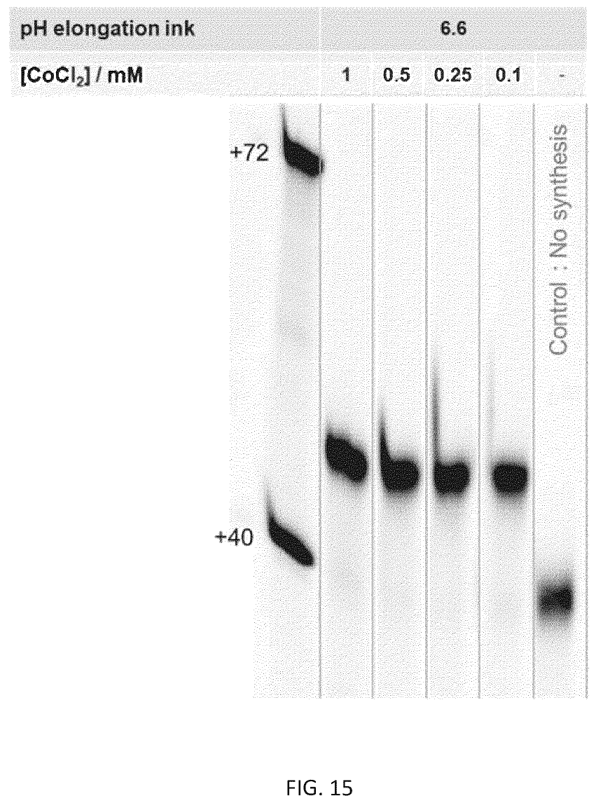

A manual synthesis was performed on the elongation ink for 5 cycles to test for the activity of the ink at different concentrations of cobalt. As shown in FIG. 15, the activity is preserved in the range of [CoCl₂] from 1 to 0.1 mM.

### Example 13: Activity of the Ink as a Function of Different Divalent Cations

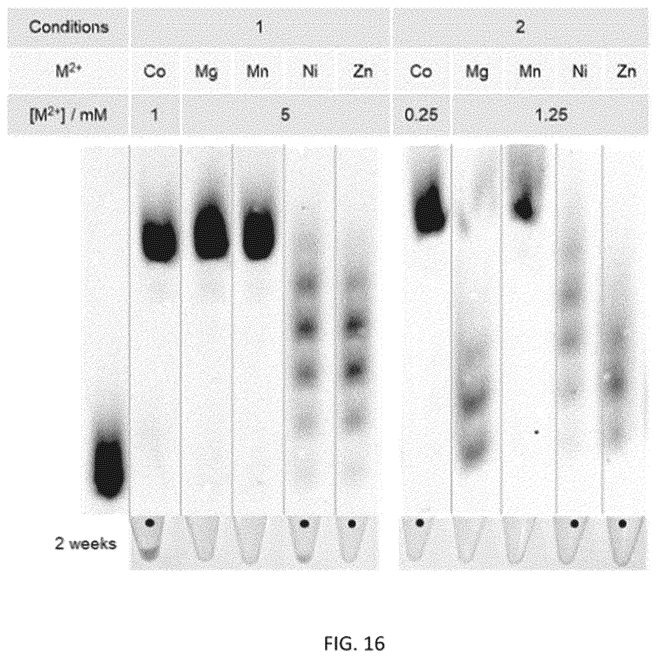

A manual synthesis was performed on elongation ink for 5 cycles at 20° C. to test the ink activity with different divalent cations at different concentrations. As shown in FIG. 16, the activity is preserved for 0.25 mM and 1 mM of Co, 1.25 mM and 5 mM of Mn, 5 mM Mg. We can also see that certain inks are less stable than other after two weeks.

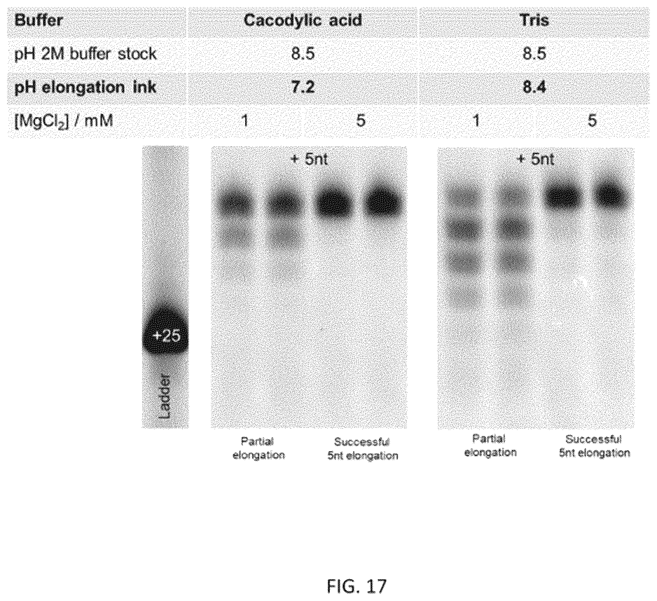

In addition, for Mg, tests were performed with different buffers and different pHs. As shown in FIG. 17, successful elongations were obtained at pHs above 6.6.

The above examples are provided to illustrate the invention but not to limit its scope. Other variants of the invention will be readily apparent to one of ordinary skill in the art and are encompassed by the appended claims. All publications, accessions, references, databases, and patents cited herein are hereby incorporated by reference for all purposes.

1. A method of enzymatically synthesizing a plurality of polynucleotides each having a predetermined sequence at reaction sites on a substrate, the method comprising:
(a) providing the substrate, wherein the substrate comprises initiators at a plurality of reaction sites, wherein each initiator has a free 3′-hydroxyl group, and wherein each polynucleotide of the plurality is assigned to a reaction site for synthesis;
(b) providing a printable reagent composition comprising a template-free polymerase, a divalent cation, a 3′-O-protected nucleoside triphosphate, and a polar organic solvent that reduces DNA melting temperature,
wherein the divalent cation:
(i) is a cobalt divalent cation (Co²⁺) at a concentration of about or between 0.1 mM to 1 mM, wherein the pH of the printable reagent composition is about or between 5.1 to 6.6,
(ii) is a manganese divalent cation (Mn²⁺) at a concentration of about or between 0.5 mM to 7 mM, wherein the pH of the printable reagent composition is about or between 6.0 to 7.0, or
(iii) is a magnesium divalent cation (Mg²⁺) at a concentration of about or between 1 mM to 20 mM, wherein the pH of the printable reagent composition is about or between 5.6 to 8.4;
(c) performing a reaction cycle comprising the steps of
(i) dispensing through one or more inkjet printhead nozzles at least one droplet of the printable reagent composition to each reaction site of the plurality, wherein the initiator or elongated fragments having free 3′-O-hydroxyls are reacted with the 3′-O-protected nucleoside triphosphate under suitable conditions for elongation by the template-free polymerase, wherein the initiator or elongated fragments are elongated by incorporation of the 3′-O-protected nucleoside triphosphate to form 3′-O-protected elongated fragments, and
(ii) dispensing through one or more inkjet printhead nozzles at least one droplet of a deprotection solution to deprotect the 3′-O-protected elongated fragments to form elongated fragments having free 3′-hydroxyls; and
(d) repeating step (c) until the plurality of polynucleotides is synthesized.

2. The method of claim 1, wherein the buffer is cacodylic acid, HEPES, Tris or MES.

3. The method of claim 1, wherein the printable reagent composition is stored at a temperature in a range from −20° C. to 4° C. prior to said dispensing.

4. The method of claim 1, wherein the printable reagent composition is filtered through a filter before said dispensing.

5. The method of claim 1, wherein the printable reagent composition is prepared by a method comprising:
(i) mixing the divalent cation, the polar organic solvent, and the buffer to form a solution;
(ii) filtering the solution through a first filter to form a filtered solution;
(iii) adding the template-free polymerase and the 3′-O-protected nucleoside triphosphate to the filtered solution to form the printable reagent composition; and
(iv) filtering the printable reagent composition through a second filter before said dispensing.

6. The method of claim 1, wherein the printable reagent composition comprises an inorganic pyrophosphatase and/or is deprived of exogenous source of pyrophosphate.

7. The method of claim 1, wherein the 3′-O-protected nucleoside triphosphate in the printable reagent composition has been purified to remove contaminating pyrophosphate.

8. The method of claim 1, wherein the printable reagent composition comprises:
(i) a first reagent composition comprising the template-free polymerase and the divalent cation and a second reagent composition comprising the 3′-O-protected nucleoside triphosphate and the polar organic solvent, or
(ii) a first reagent composition comprising the template-free polymerase and the 3′-O-protected nucleoside triphosphate and a second reagent composition comprising the divalent cation and the polar organic solvent.

9. The method of claim 8, wherein the first reagent composition and the second reagent composition:
(i) are dispensed separately through the one or more inkjet printhead nozzles, or
(ii) are mixed prior to said dispensing.

10. The method of claim 8, wherein the first reagent composition and the second reagent composition are stored at a temperature in a range from −20° C. to 4° C. prior to said dispensing.

11. A printable reagent composition, comprising:
a template-free polymerase, a divalent cation, wherein the divalent cation:
(i) is a cobalt divalent cation (Co²⁺) at a concentration of about or between 0.1 mM to 1 mM, wherein the pH of the printable reagent composition is about or between 5.1 to 6.6;
(ii) is a manganese divalent cation (Mn²⁺) at a concentration of about or between 0.5 mM to 7 mM, wherein the pH of the printable reagent composition is about or between 6.0 to 7.0; or
(iii) is a magnesium divalent cation (Mg²⁺) at a concentration of about or between 1 mM to 20 mM, wherein the pH of the printable reagent composition is about or between 5.6 to 8.4;
a buffer,
a 3′-O-protected nucleoside triphosphate, and
a polar organic solvent that reduces DNA melting temperature.

12. The printable reagent composition of claim 11, wherein the buffer is cacodylic acid, HEPES, Tris or MES.

13. The printable reagent composition of claim 11, wherein the printable reagent composition comprises an inorganic pyrophosphatase and/or the printable reagent composition is deprived of exogenous source of pyrophosphate.

14. The printable reagent composition of claim 11, wherein the 3′-O-protected nucleoside triphosphate in the printable reagent composition has been purified to remove contaminating pyrophosphate.

15. The printable reagent composition of claim 11, wherein the printable reagent composition comprises 20 μM terminal deoxynucleotidyl transferase, 500 μM 3′-O-protected nucleoside triphosphate, 0.25 mM CoCl₂, 15% DMSO, 50 mM O-benzylhydroxylamine hydrochloride, 10% glycerol, and 0.05% polyoxyethylene (20) sorbitan monolaurate.

16. A set of printable reagent compositions, comprising:
(i) a first reagent composition comprising a template-free polymerase and a divalent cation; and a second reagent composition comprising a 3′-O-protected nucleoside triphosphate and a polar organic solvent, or
(ii) a first reagent composition comprising a template-free polymerase and a 3′-O-protected nucleoside triphosphate; and a second reagent composition comprising a divalent cation, and a polar organic solvent, wherein the divalent cation is a cobalt divalent cation (Co²⁺), a manganese divalent cation (Mn²⁺) or a magnesium divalent cation (Mg²⁺).

17. The method of claim 1, wherein the polar organic solvent is dimethyl sulfoxide (DMSO), a betaine, or methanol at a concentration of about or between 5 volume/volume percent to 30 volume/volume percent.

18. The method of claim 5, wherein the template-free polymerase is selected from the group consisting of: a terminal deoxynucleotidyl transferase, a translesion DNA polymerase of type q (eta), a translesion DNA polymerase of type C (zeta), a polynucleotide phosphorylase (PNPase), a template-independent RNA polymerase, a terminal transferase, a template-independent DNA polymerase, a reverse transcriptase, and a 9° N DNA polymerase.

19. The composition of claim 11, wherein the 3′-O-protected nucleoside triphosphate comprises a 3′-aminooxy protecting group at a concentration of 100 μM to 2000 μM.

20. The composition of claim 12, wherein the buffer comprises 0.5M cacodylic acid.
